SEQUENCE LISTING (134 sequences)

SEQ ID NO: 1 - protein, 844 aa, Synthetic construct
MKLSTSGMGQQAHEGENKCLNSELWHACAGPLVCLPTVGSRVVYFPQGHSEQVAATTNKEVDIHIPNYPNLPPQLICQLHNVTMHADVETDEVYAQMTLQPLTLQEQKDTYLPVELGIPSRQPTNYFCKTLTASDTSTHGGFSVPRRAAEKVFPPLDFSQTPPCQELIARDLHDIEWKFRHIFRGQPKRHLLTTGWSVFVSAKRLVAGDSVLFIWNEKNQLFLGIRRATRPQTVMPSSVLSSDSMHIGLLAAAAHAASTNSCFIVFFNPRASPSEFVIPLSKYIKAVYHTRVSVGMRFRMLFETEESSVRRYMGTITGIGDLDPVRWANSHWRSVKVGWDESTAGERQPRVSLWEIEPLTTFPMYPSLFPLRLKRPWYPGTSSFQENNSEAINGMTWLRGESSEQGPHLLNLQSFGGMFPWMQQRVDPTMLRNDLNQQYQAMLASGLQNFGSGDLMKQQLMQFPQPVQYVQHAGSVNPQLQQQQQQQETMQQTIHHHMLPAQTQDNLQRQQQQHVSNQTEEQSHQHSYQDAYQIPNSQLQQKQPSNVPSPSFSKPDIADPSSKFSASIAPSGMPTALGSLCSEGTTNFLNFNIIGQQPVIMEQQQQQKSWMAKFANSQLNMGSSSPSLSGYGKETSNSQETCSLDAQNQSLFGANVDSSGLLLPTTVSNVATTSIDADISSMPLGTSGFPNPLYSYVQDSTDLLHNVGQADAQTVPRTFVKVYKSASLGRSLDITRFNSYHELRQELGQMFGIEGFLENPQRSGWQLVFVDRENDVLLLGDDPWEEFVNNVWYIKILSPEDVQKLGKEEVGSLNRGPPERMSSNNSADGRDFMSGLPSIGSLDY

SEQ ID NO: 2 - DNA, 2535 bp, Synthetic construct
  source 1..2535: mol_type = other DNA
atgaagctttcaacatcaggaatgggtcagcaagctcatgaaggagagaacaagtgtttgaattcagaactatggcatgcttgtgctggtccccttgtttgtctaccaacggtagggagtcgagtggtttactttcctcagggtcacagtgaacaggttgcggcgacaactaataaagaagtcgatattcacatacctaattacccgaacttgccaccacagttgatctgtcaactccacaatgtcacaatgcatgcagatgttgaaacggatgaagtatatgctcagatgacattgcaacccttgacactgcaagaacaaaaggacacgtatcttcctgttgaattgggtattcctagcaggcagcctactaattatttttgcaagacactcactgcaagtgataccagtacgcatggcggcttttctgttcctcgtcgtgctgcagagaaagttttccctcctttggatttctcacagacaccaccctgtcaagaattaattgcgagggatctgcatgacatcgaatggaaattcaggcatattttccgaggacagcctaagcggcatcttctgacgactggctggagtgtgtttgttagtgctaagagacttgttgccggagattcagttcttttcatctggaatgagaaaaatcagctttttttgggaattcgtcgtgcaactcgacctcagactgtgatgccatcatctgttctgtctagcgacagcatgcacattggattacttgctgctgctgctcatgctgcctctaccaatagctgtttcattgttttctttaacccaagggctagcccatccgagtttgttataccactttcaaaatacatcaaagctgtgtatcacacacgtgtttctgttggaatgcgtttccggatgctatttgagactgaagaatcaagtgttcgaaggtacatgggcacaattactggcattggtgacttagatccagttcgctgggccaactctcactggcggtctgtcaaggttggttgggatgagtcaacggcaggcgagaggcaacctagggtttcactatgggagatagagcctttgactacttttccaatgtatccatctttgttccctcttaggctaaagcggccttggtatccaggaacttcatcttttcaagaaaataacagcgaagctattaatggaatgacatggttgagaggggaaagtagtgagcaaggaccacatctactgaatcttcaatcttttggtggcatgttcccctggatgcaacaaagagttgatccaacaatgctccgaaatgatcttaaccagcagtatcaagctatgctggctagcggtttgcaaaattttgggagcggagatctgatgaaacaacaactgatgcagtttccacagcccgtccaatatgttcagcatgcaggcagtgttaatcctcaactgcagcagcagcaacaacaacaagaaacaatgcagcagacaattcatcaccatatgttgcctgcacaaactcaagataaccttcaaaggcaacaacagcaacacgttagcaatcagacagaggagcaatctcatcaacattcttaccaggatgcgtaccaaataccaaacagccagctccagcagaagcaaccatcaaatgttccttctccatcattttcaaagccagatatagcagatccaagctccaagttctcggcatccattgctccatcaggcatgccaacagcgctgggttctttatgttcggaaggaactactaactttttgaatttcaatataattggtcagcagcctgtgatcatggagcagcagcagcagcagaaatcttggatggcaaaattcgcaaattcacaattgaacatgggctccagttcaccctctctctctggatatgggaaagaaacttccaattcacaggaaacatgtagtctagatgcccagaatcaatctctttttggtgctaatgttgattcttcagggcttctcctccctacaactgtgtctaacgtcgctactacatcaattgatgctgatatatcctctatgccactagggacttctggatttccgaatcccttgtatagttatgtgcaagattctactgacttgttgcataatgtagggcaagctgatgcacaaactgtgccccgtacatttgtcaaggtttacaaatcagcgtcccttgggaggtcattggacatcactcggttcaatagctatcatgagctacgacaggaacttggacagatgttcgggatcgaagggtttcttgaaaaccctcaaagatcaggctggcagcttgtatttgttgacagggagaatgatgtccttctccttggagacgatccgtgggaggaatttgtcaataatgtttggtacatcaaaattctttcacccgaggatgtgcagaaactggggaaagaggaggttggatccctaaaccgcggtccacctgaaaggatgagcagtaataatagtgctgatggtcgagatttcatgtccggacttccatctataggatcacttgattactga

SEQ ID NO: 3 - DNA, 50 bp, Synthetic construct
  source 1..50: mol_type = other DNA
gtcgaagtagtgattgcaagtgataccagtacgcagttttagagctagaa

SEQ ID NO: 4 - DNA, 50 bp, Synthetic construct
  source 1..50: mol_type = other DNA
ttctagctctaaaactgcgtactggtatcacttgcaatcactacttcgac

SEQ ID NO: 5 - DNA, 50 bp, Synthetic construct
  source 1..50: mol_type = other DNA
gtcgaagtagtgattgcaagtgatactagcacgcagttttagagctagaa

SEQ ID NO: 6 - DNA, 50 bp, Synthetic construct
  source 1..50: mol_type = other DNA
ttctagctctaaaactgcgtgctagtatcacttgcaatcactacttcgac

SEQ ID NO: 7 - DNA, 46 bp, Synthetic construct
  source 1..46: mol_type = other DNA
gactctagacacggggtggtttaagctttcgttgaacaacggaaac

SEQ ID NO: 8 - DNA, 38 bp, Synthetic construct
  source 1..38: mol_type = other DNA
cttgtttaaacgtttaaacaaaaaaagcaccgactcgg

SEQ ID NO: 9 - DNA, 39 bp, Synthetic construct
  source 1..39: mol_type = other DNA
tttgtttaaacgtttaaacaagctttcgttgaacaacgg

SEQ ID NO: 10 - DNA, 44 bp, Synthetic construct
  source 1..44: mol_type = other DNA
agcaactcattacaacttgtttcaaaaaaagcaccgactcggtg

SEQ ID NO: 11 - DNA, 27 bp, Synthetic construct
  source 1..27: mol_type = other DNA
ggtccttgttattgtcgaccgttttcg

SEQ ID NO: 12 - DNA, 42 bp, Synthetic construct
  source 1..42: mol_type = other DNA
tcagacccgggatttttaaacacccttaaattatgattatcc

SEQ ID NO: 13 - DNA, 36 bp, Synthetic construct
  source 1..36: mol_type = other DNA
gtacgtcgacatgaagctttcaacatcaggaatggg

SEQ ID NO: 14 - DNA, 36 bp, Synthetic construct
  source 1..36: mol_type = other DNA
gtacgagctctcagtaatcaagtgatcctatagatg

SEQ ID NO: 15 - DNA, 61 bp, Synthetic construct
  source 1..61: mol_type = other DNA
gatccgtacccatacgatgttccagattacgcttacccatacgatgttccagattacgctg

SEQ ID NO: 16 - DNA, 61 bp, Synthetic construct
  source 1..61: mol_type = other DNA
aattcagcgtaatctggaacatcgtatgggtaagcgtaatctggaacatcgtatgggtacg

SEQ ID NO: 17 - DNA, 43 bp, Synthetic construct
  source 1..43: mol_type = other DNA
atcaggggcccgggatggactacaaagaccatgacggtgatta

SEQ ID NO: 18 - DNA, 41 bp, Synthetic construct
  source 1..41: mol_type = other DNA
attcgtcgacagcgtaatctggaacatcgtatgggtaagcg

SEQ ID NO: 19 - DNA, 36 bp, Synthetic construct
  source 1..36: mol_type = other DNA
agctgtcgacatgaagctttcaacatcaggaatggg

SEQ ID NO: 20 - DNA, 34 bp, Synthetic construct
  source 1..34: mol_type = other DNA
agctgagctcagtactccagcgatccaagagatg

SEQ ID NO: 21 - DNA, 27 bp, Synthetic construct
  source 1..27: mol_type = other DNA
ggactaaaagcctcactacggtccgtg

SEQ ID NO: 22 - DNA, 41 bp, Synthetic construct
  source 1..41: mol_type = other DNA
agctcccgggaattatattttatttatttatttaaaaaccc

SEQ ID NO: 23 - DNA, 29 bp, Synthetic construct
  source 1..29: mol_type = other DNA
aaacttaattaatttaaatcccgggatcc

SEQ ID NO: 24 - DNA, 33 bp, Synthetic construct
  source 1..33: mol_type = other DNA
tcgaggatcccgggatttaaattaattaagttt

SEQ ID NO: 25 - DNA, 47 bp, Synthetic construct
  source 1..47: mol_type = other DNA
gtttaaacttaattaatttatggactacaaagaccatgacggtgatt

SEQ ID NO: 26 - DNA, 44 bp, Synthetic construct
  source 1..44: mol_type = other DNA
tcgaggatcccgggattttcagtactccagcgatccaagagatg

SEQ ID NO: 27 - DNA, 47 bp, Synthetic construct
  source 1..47: mol_type = other DNA
ctctagacacggggtggtttctagacttttaaattttgaagtgttat

SEQ ID NO: 28 - DNA, 51 bp, Synthetic construct
  source 1..51: mol_type = other DNA
gtccataaattaattaagtttaattatattttatttatttatttaaaaacc

SEQ ID NO: 29 - DNA, 45 bp, Synthetic construct
  source 1..45: mol_type = other DNA
tcgaggatcccgggatttttagtttgggaattctgatgttgatcc

SEQ ID NO: 30 - DNA, 47 bp, Synthetic construct
  source 1..47: mol_type = other DNA
ctctagacacggggtggtttctctatcatgacttctcgcgtcatttc

SEQ ID NO: 31 - DNA, 49 bp, Synthetic construct
  source 1..49: mol_type = other DNA
gtccataaattaattaagtttaaaaacctctgtgattccctttacatca

SEQ ID NO: 32 - DNA, 35 bp, Synthetic construct
  source 1..35: mol_type = other DNA
gtacgtcgacatgaaagcgccatcaaacggatatc

SEQ ID NO: 33 - DNA, 38 bp, Synthetic construct
  source 1..38: mol_type = other DNA
gtatccccgggtcaattaaatgatgcagctgagttgtc

SEQ ID NO: 34 - DNA, 74 bp, Synthetic construct
  source 1..74: mol_type = other DNA
cgggatggactacaaagaccatgacggtgattataaagatcatgacatcgattacaaggatgacgatgacaagg

SEQ ID NO: 35 - DNA, 82 bp, Synthetic construct
  source 1..82: mol_type = other DNA
tcgaccttgtcatcgtcatccttgtaatcgatgtcatgatctttataatcaccgtcatggtctttgtagtccatcccgggcc

SEQ ID NO: 36 - DNA, 35 bp, Synthetic construct
  source 1..35: mol_type = other DNA
gcacgtcgacgccctatcttagggtgtctgtaaag

SEQ ID NO: 37 - DNA, 47 bp, Synthetic construct
  source 1..47: mol_type = other DNA
atgcgagctccccgggttcagcaaaaaatccacttaagttgaattac

SEQ ID NO: 38 - DNA, 32 bp, Synthetic construct
  source 1..32: mol_type = other DNA
ataagtcgaccgtaggtgggtgggtgggggtg

SEQ ID NO: 39 - DNA, 45 bp, Synthetic construct
  source 1..45: mol_type = other DNA
cggagagctcccgggcacaattacagatcactctaataatcctgc

SEQ ID NO: 40 - DNA, 38 bp, Synthetic construct
  source 1..38: mol_type = other DNA
gtacgagctcttagggcacagtttgtgcatcagcttgc

SEQ ID NO: 41 - DNA, 40 bp, Synthetic construct
  source 1..40: mol_type = other DNA
gtacgagctcttaggttgcatttggtggatcaatttgtcc

SEQ ID NO: 42 - DNA, 39 bp, Synthetic construct
  source 1..39: mol_type = other DNA
tcaggtcgacatggactacaaagaccatgacggtgatta

SEQ ID NO: 43 - DNA, 35 bp, Synthetic construct
  source 1..35: mol_type = other DNA
ctaggtcgacatgtcatctctcagtagagagcttg

SEQ ID NO: 44 - DNA, 35 bp, Synthetic construct
  source 1..35: mol_type = other DNA
aaacgtcgacatcctttacaagaatttcaacaccc

SEQ ID NO: 45 - DNA, 24 bp, Synthetic construct
  source 1..24: mol_type = other DNA
tgctgttgcattttcagcaagaac

SEQ ID NO: 46 - DNA, 25 bp, Synthetic construct
  source 1..25: mol_type = other DNA
aagacagtggaccagttatatcacc

SEQ ID NO: 47 - DNA, 22 bp, Synthetic construct
  source 1..22: mol_type = other DNA
ctctgtgttggatttcaggaag

SEQ ID NO: 48 - DNA, 26 bp, Synthetic construct
  source 1..26: mol_type = other DNA
cgacgagggacagaaaagccaccacg

SEQ ID NO: 49 - DNA, 24 bp, Synthetic construct
  source 1..24: mol_type = other DNA
gagaagcaattggagcagagtgtc

SEQ ID NO: 50 - DNA, 24 bp, Synthetic construct
  source 1..24: mol_type = other DNA
gaggctctcctagctcataacaac

SEQ ID NO: 51 - DNA, 23 bp, Synthetic construct
  source 1..23: mol_type = other DNA
gaagaagaggaggaggccaattg

SEQ ID NO: 52 - DNA, 22 bp, Synthetic construct
  source 1..22: mol_type = other DNA
gggaagctgacgactgggctac

SEQ ID NO: 53 - DNA, 22 bp, Synthetic construct
  source 1..22: mol_type = other DNA
gccctggagttgtttcttgtgc

SEQ ID NO: 54 - DNA, 21 bp, Synthetic construct
  source 1..21: mol_type = other DNA
accctcaacactcaacccgag

SEQ ID NO: 55 - DNA, 23 bp, Synthetic construct
  source 1..23: mol_type = other DNA
gtggtgtacatgcttgtccaggg

SEQ ID NO: 56 - DNA, 24 bp, Synthetic construct
  source 1..24: mol_type = other DNA
cagtccagttttcgacagctccgc

SEQ ID NO: 57 - DNA, 20 bp, Synthetic construct
  source 1..20: mol_type = other DNA
gagaggccttctgctgtgaa

SEQ ID NO: 58 - DNA, 23 bp, Synthetic construct
  source 1..23: mol_type = other DNA
gtctcatgattggcatagaagag

SEQ ID NO: 59 - DNA, 24 bp, Synthetic construct
  source 1..24: mol_type = other DNA
tggatctgtctatgatagtgagag

SEQ ID NO: 60 - DNA, 23 bp, Synthetic construct
  source 1..23: mol_type = other DNA
cccatccctcacccaaaattctc

SEQ ID NO: 61 - DNA, 21 bp, Synthetic construct
  source 1..21: mol_type = other DNA
cagcatcagcatctaacaacg

SEQ ID NO: 62 - DNA, 22 bp, Synthetic construct
  source 1..22: mol_type = other DNA
gatgaatgcacctctcgagaaa

SEQ ID NO: 63 - DNA, 23 bp, Synthetic construct
  source 1..23: mol_type = other DNA
gatgtagatgcagatccaagtcc

SEQ ID NO: 64 - DNA, 19 bp, Synthetic construct
  source 1..19: mol_type = other DNA
ggcaataggtggagggatc

SEQ ID NO: 65 - DNA, 41 bp, Synthetic construct
  source 1..41: mol_type = other DNA
gtctgagaagacatcgcaacccacggttcaaatccaagacg

SEQ ID NO: 66 - DNA, 21 bp, Synthetic construct
  source 1..21: mol_type = other DNA
ggttgctaaatcagcccatat

SEQ ID NO: 67 - DNA, 24 bp, Synthetic construct
  source 1..24: mol_type = other DNA
cctgtctaaaagcctagagacaac

SEQ ID NO: 68 - DNA, 21 bp, Synthetic construct
  source 1..21: mol_type = other DNA
cctatggctcctccattgggc

SEQ ID NO: 69 - DNA, 24 bp, Synthetic construct
  source 1..24: mol_type = other DNA
catagggtacacctagctagatgt

SEQ ID NO: 70 - DNA, 22 bp, Synthetic construct
  source 1..22: mol_type = other DNA
gcttatacccgtagcacggtac

SEQ ID NO: 71 - DNA, 23 bp, Synthetic construct
  source 1..23: mol_type = other DNA
cgtcccaatgtctcattcagtgg

SEQ ID NO: 72 - DNA, 21 bp, Synthetic construct
  source 1..21: mol_type = other DNA
gcgccatccaaagcggaccca

SEQ ID NO: 73 - DNA, 51 bp, Synthetic construct
  source 1..51: mol_type = other DNA
gtcgaagtagtgattgtgtgccccgtacatttgtcagttttagagctagaa

SEQ ID NO: 74 - DNA, 51 bp, Synthetic construct
  source 1..51: mol_type = other DNA
ttctagctctaaaactgacaaatgtacggggcacacaatcactacttcgac

SEQ ID NO: 75 - DNA, 51 bp, Synthetic construct
  source 1..51: mol_type = other DNA
gtcgaagtagtgattgatgtccggacttccatctatgttttagagctagaa

SEQ ID NO: 76 - DNA, 51 bp, Synthetic construct
  source 1..51: mol_type = other DNA
ttctagctctaaaacatagatggaagtccggacatcaatcactacttcgac

SEQ ID NO: 77 - DNA, 24 bp, Synthetic construct
  source 1..24: mol_type = other DNA
gggctccagttcaccctctctctc

SEQ ID NO: 78 - DNA, 24 bp, Synthetic construct
  source 1..24: mol_type = other DNA
gtctacatcacatatgtcctgatc

SEQ ID NO: 79 - DNA, 22 bp, Synthetic construct
  source 1..22: mol_type = other DNA
cctctatgccactagggacttc

SEQ ID NO: 80 - DNA, 26 bp, Synthetic construct
  source 1..26: mol_type = other DNA
cctaaacaacagaactagaaccttta

SEQ ID NO: 81 - DNA, 24 bp, Synthetic construct
  source 1..24: mol_type = other DNA
gcacaaactgtgccccgtaccttt

SEQ ID NO: 82 - DNA, 22 bp, Synthetic construct
  source 1..22: mol_type = other DNA
gtccaatgacctcccaagggac

SEQ ID NO: 83 - protein, 842 aa, Synthetic construct
MKLSTSGMGQQAHEGGEKKCLNSELWHACAGPLVCLPTVGSRVVYFPQGHSEQVAATTNKEVDAHIPNYPNLSPQLICQLHNVTMHADVETDEVYAQMTLQPLTPEEQKDTYLPVEFGIPSKQPTNYFCKTLTASDTSTHGGFSVPRRAAEKVFPPLDFSQTPPAQELIARDLHDVEWKFRHIFRGQPKRHLLTTGWSVFVSAKRLVAGDSVLFIWNEKNQLLLGIRRAVRPQTVMPSSVLSSDSMHIGLLAAAAHAAATNSCFNVFFNPRASPSEFVIPLSKYIKAVYHTRVSVGMRFRMLFETEESSVRRYMGTITGIGDLDPVRWANSHWRSVKVGWDESTAGERQPRVSLWEIEPLTTFPMYPSLFPLRLKRPFYQGTSSYQDSNNEAINRMSWLRGNAGELGHHSMNLQSFGMLPWMQQRVDSTILPNDINQHYQAMLATGLQSFGSGDLLKQQLMQFQQPVQYLQHASTENSILHQQQQQQQQIMQQAVHQHMLPAQTQMLSENLQRQSQHQSNNQSEEQAHQHTYQEAFQLPHDQLQQRQPSNVTSPFLKADFADLTSKFSASVAPSGVQNMLGSLCSEGSNNSLNINRTGQSVIIEQSPQQSWMSKFTESQLNTCSNSSSLPTYGKDTSNPRGNCSLDSQNQALFGANIDSSGHLLPTTVSNVTTTCADMSLMPLGASGYQNSLYGYVQDSSELLHNAGQIDPPNATHTFVKVYKSGCVGRSLDITQFHSYHELRRELGQMFGIEGFLEDPQRSGWQLVFVDRENDILLLGDDPWEAFVNNVWYIKILSPEDVQKLGKEEAESLNRGAVERMSSTNADDRDLISGMPSLGSLEY

SEQ ID NO: 84 - protein, 811 aa, Synthetic construct
MKLSTSGLGQQGHEGEKCLNSELWHACAGPLVSLPSSGSRVVYFPQGHSEQVAATTNKEVDGHIPNYPSLPPQLICQLHNVTMHADVETDEVYAQMTLQPLTPEEQKETFVPIELGIPSKQPSNYFCKTLTASDTSTHGGFSVPRRAAEKVFPPLDYTLQPPAQELIARDLHDVEWKFRHIFRGQPKRHLLTTGWSVFVSAKRLVAGDSVIFIRNEKNQLFLGIRHATRPQTIVPSSVLSSDSMHIGLLAAAAHASATNSCFTVFFHPRASQSEFVIQLSKYIKAVFHTRISVGMRFRMLFETEESSVRRYMGTITGISDLDSVRWPNSHWRSVKVGWDESTAGERQPRVSLWEIEPLTTFPMYPSLFPLRLKRPWHAGTSSLPDGRGDLGSGLTWLRGGGGEQQGLLPLNYPSVGLFPWMQQRLDLSQMGTDNNQQYQAMLAAGLQNIGGGDPLRQQFVQLQEPHHQYLQQSASHNSDLMLQQQQQQQASRHLMHAQTQIMSENLPQQNMRQEVSNQPAGQQQQLQQPDQNAYLNAFKMQNGHLQQWQQQSEMPSPSFMKSDFTDSSNKFATTASPASGDGNLLNFSITGQSVLPEQLTTEGWSPKASNTFSEPLSLPQAYPGKSLALEPGNPQNPSLFGVDPDSGLFLPSTVPRFASSSGDAEASPMSLTDSGFQNSLYSCMQDTTHELLHGAGQINSSNQTKNFVKVYKSGSVGRSLDISRFSSYHELREELGKMFAIEGLLEDPLRSGWQLVFVDKENDILLLGDDPWESFVNNVWYIKILSPEDVHQMGDHGEGSGGLFPQNPTHL

SEQ ID NO: 85 - protein, 661 aa, Synthetic construct
MVPVWMWKEGQPKRHLLTTGWSVFVSAKRLVAGDSVLFIWNDKNQLLLGIRRAIRPQTVMPSSVLSSDSMHIGLLAAAAHAAATNSCFTVFFNPRASPSEFVIPLSKYIKAVYHTRVSVGMRFRMLFETEESSVRRYMGTITGIGDLDPVRWANSHWRSVKVGWDESTAGERQPRVSLWEIEPLTTFPMYPSLFPLRLKRPLYPGTSSYQDSNNEAINRMTWLRGNTSELGPHSMNLQSFGMLPWMQQRVDSRIIPNDINQHYQAMLATGLQSFGSGDLLKQQLVQFQQPAQYLQHASTDNSILHQQQIMLQAVHQHMQPAQTQMLSENLQRQSQQQSNNQLEEQAHQHAYQEAFQIPHNQLQQRPSSNVSSQFLKADFADLNSKFSASVAPSGVQNMLGSLCSEGSSNSLNINRTGQCVIMEQPPQQSWMSKFAESQLNTCSNSSPLPTYEKDTSNPRENCSLDSQNQVLFVANVDSSGHLLPTRVSNVTTTCADMSAMPLGASGYQNSLYGYAQDSSELLHNAGQIDPPNATHTFVKVYKSGCVGRSLDITQFHSYHELRRELGQMFGIEGFLEDPQRSGWQLVFVDRENDILLLGDDPWEAFVNNVWYIKILSPEDVQKLGKEEAESLNRGAVERMSSSSVDDRDLISGMPSLGSLEY

SEQ ID NO: 86 - protein, 891 aa, Synthetic construct
MRVSSSGFNPQPEEATGEKKCLNSELWHACAGPLVSLPPVGSRVVYFPQGHSEQVAASTNKEVDAHIPNYPGLPPQLICQLHNLTMHADVETDEVYAQMTLQPLSPQEQKDVCLLPAELGIPSKQPTNYFCKTLTASGTSTHGGFSVPRRAAEKVFPPLDYSQQPPCQELIAKDLHGNEWKFRHIFRGQPKRHLLTTGWSVFVSAKRLVAGDAVIFIWNENNQLLLGIRRANRPQTVMPSSVLSSDSMHIGLLAAAAHAAATNSRFTIFYNPRASPSEFVIPLAKYVKAVYHTRISVGMRFRMLFETEESSVRRYMGTITGISDLDPVRWPNSHWRSVKVGWDESTAGDRQPRVSLWEIEPLTTFPMYPSPFSLRLKRPWPSGLPSLTGFPNGDMAMNSPLSWLRGDMGDQGMQSLNFQGFGVTPFMQPRMDASLLGLQPDILQTMAALDPSKLANQSLMQFQQSIPNSSASLSQSQMLQPSHSHQNLIQGFSENHLISQAQMLQQQLQRRQNFNDQQQLLQPQLQQHQEVNSQFQHQQRTKAISSLSQMASVTQPHLSHLPVLSSTGSQQTFSDMLGTHVNSSSNSNMQSLLSSFSRDGAPAVLNMHETHPLVSSSSSSKRIALESQLPSRVTPFVLSQPENVIAPNTKVSDLSSLLPPFPGRESFSDYKGAEDSQSNALYGFTDSLNILQTGMSNMKGSSGDNGSLSIPYAISTFTSTVGNEYPLNSDMTASSCVDESGFLQSSENGDQANQTNRIFVKVQKSGSFGRSLDISKFSSYHELRSELARMFGLEGLLEDPERSGWQLVIVDRENDVLLLGDDPWQEFVNNVWYIKILSPYEVQQMGKEGLDLLNGVRTQRLPGNVNGCDDYMNQKGSRNTMNGIPLGSLDY

SEQ ID NO: 87 - protein, 854 aa, Synthetic construct
MKLSTSGFGQQDHEGGEKKCLNSELWHACAGPLVSLPTAGTRVVYFPQGHSEQVAATTNKEVDGHIPNYPNLPPQLICQLHNVTMHADVETDEVYAQMTLQPLTAQEQKDTFLPMELGIPSRQPTNYFCKTLTASDTSTHGGFSVPRRAAEKVFPPLDFSQQPPAQELIAKDLHDIEWKFRHIFRGQPKRHLLTTGWSVFVSAKRLVAGDSVLFIWNEKNQLLLGIRRATRPQTVMPSSVLSSDSMHIGLLAAAAHAAATNSCFTVFYNPRASPSEFVIPLTKYVKAVFHTRVSVGMRFRMLFETEESSVRRYMGTITGISDLDPVRWPNSHWRSVKVGWDESTAGERQPRVSLWEIEPLTTFPMYPSLFPLRLKRPWHPGVSSVHDNREDASNGLMWLRGGVGEQGLHSLNLQSVSSLPWLQQRLDSSMFGNDHNQQYQAMLAAGMPNLGGVDMLRQQIMHLQQPFQYIPQAGFHNSLLQMQQQQQQQQQQQQQQLVQHSMPQNILQAPSQVMAENLPQHILQQTLQNQPEDLPNQQQHTYHDTIQVQSNQFHQGGHSNVPSPTFPRTDLMDSNTSYSESITSRRNILASSCAEGTGNLSTIYRSGQSILTEHLPQQSPVSKNAHSQVDAHPNSMSFPPFSGRDSILELRNCNSDSPSPTLFGVNIDSSGLLLPSNVPTYTSPSIGPDSSSMPLGDSGFQNSLYSCVQDSSELLHNSGQVDPSNPTRTFVKVYKTGSVGRSLDISRFSSYQELREELAQMFGIEGQLVEDPRRSGWQLVFVDRENDVLLLGDDPWEAFVNNVGFIKILSPEDFQKLGEQAIESFNPIVGQRLTSGGNEAGNVSGLPSVGSLEY

SEQ ID NO: 88 - protein, 843 aa, Synthetic construct
MKLSTSGLGQQDHEGGGGGGVEKKCLNSELWHACAGPLVSLPTPGTRVVYFPQGHSDQVAATTNKQVDAHIPNYPNLPPQLICQLHNVTMHADVETDEVYAQMTLQPLTPQEQKETFLPMELGLPSKQPTNYFCKTLTASDTSTHGGFSVPRRAAEKVFPPLDFTLQPPAQELIARDLHDVEWKFRHIFRGQPKRHLLTTGWSVFVSAKRLVAGDSVLFIWNEKNQLFLGIRRATRPQTVMPSSVLSSDSMHIGLLAAAAHAASTNSCFTVFYNPRASPSEFVIPLSKYIKAVFHTRVSVGMRFRMLFETEESSVRRYMGTITGISDLDPVRWPNSHWRSVKVGWDESTSGERQPRVSLWEIEPLTTFPMYPSLFPLRLKRPWIPGASSMHDNRDEANSLMWLRGGAGEQGLQSMNFQNVGMFPWMQQRLDSTLIGNDHNQQYQSMLAAGLQNLGSGDQLRQQMMHFQQPFQYVQQSGSHNPMLQLQQQAIQQSIPHNILQGQPQVSMENMHQQLLQQQFNNQTDAQVQQQQNAYQDALKVQNEQLHQSHVPSPSFTKPDFSDSSTNFSTSTPRQNVLGTLCPEGRGNLLSMSSAGHSMPTEQSPQQSWDPKYAHGQANAFSNSMSFPPFNGKDNAVEQENLNSDTHNPTLFGVNIESSGLLFPNTVPSFATSSNDADISMPLGNTGFQSSLYGCMQDSSELLHGAGQVDPPTPNNCTFVKVYKSGSVGRSIDISRFSSYHELREELGQMFGIEGKLEYRLRSGWQLVFVDREDDVLLLGDDPWESFVNNVWYIKILSPEDVHKMGHQAVESFSPNTGQRLNSGGGEGQDIVSGLPSLGSLEY

SEQ ID NO: 89 - protein, 811 aa, Synthetic construct
MKLSTSGLCQQGHEGDNKCLNSELWHACAGPLVSLPTVGTRVVYFPQGHSEQVAATTNKEVDSHIPNYPNLPPQLICQLHNVTMHADVETDEVYAQMTLQPLSPEEQKDTFVPIELGIPSKQPTNYFCKTLTASDTSTHGGFSVPRRAAEKVFPSLDFSLQPPAQELIARDLHDVEWKFRHIFRGQPKRHLLTTGWSVFVSAKRLVAGDSVLFIWNEKNQLLLGIRRAIRPPTVMPSSVLSSDSMHIGLLAAAAHAAATNSCFTVFFNPRASPSEFVIPLTKYVKAVFHTRVSVGMRFRMLFETEESSVRRYMGTITGISDLDPVRWSNSHWRSVKVGWDESTAGERQPRVSLWEIEPLTTFPMYPSLFPLRLKRPWHPSTSSFNDNRDETASGLNWLRGGTGEQGLTTLNFQSLGMFPWMQQRVEPSFLGNDHNQQYQAMLAAGMQSGDPVRQQFMQLQQPFQYLQQSGSQNPLQLKQQQHLLQQLNSQAEDRAQQQQQPQQHMYHDALQIRTDELLQRQQSNLPSPSFSKANFMDSSTEISVSISPMQNMLGSLPEGSGNLLNFSGAGPSMLRQQFPQQSLGSKYEPSQVRDFVHSMSLPSSYNGKDAAVGTENCNTDSQNSVVFGVHIDSSGLLLPTTVSSFTTSVDPGVSSMPLGDSGFHNSMYGCMQDSSELLHNVGQIDQLTPTRTFVKVYKSGSVGRSLDISRFSSYNELREELGQMFGIEGKFEDPLRSGWQLVFVDRENDVLLLGDDPWEAFVSNVWYIKILSPEDVQKMGEQGVESFSPSSGQRANSRGNCGRDPVGSLEY

SEQ ID NO: 90 - protein, 100 aa, Synthetic construct
AQTVPRTFVKVYKSASLGRSLDITRFNSYHELRQELGQMFGIEGFLENPQRSGWQLVFVDRENDVLLLGDDPWEEFVNNVWYIKILSPEDVQKLGKEEVG

SEQ ID NO: 91 - protein, 100 aa, Synthetic construct
PPNATHTFVKVYKSGCVGRSLDITQFHSYHELRRELGQMFGIEGFLEDPQRSGWQLVFVDRENDILLLGDDPWEAFVNNVWYIKILSPEDVQKLGKEEAE

SEQ ID NO: 92 - protein, 100 aa, Synthetic construct; identical to SEQ ID NO: 91

SEQ ID NO: 93 - protein, 100 aa, Synthetic construct
SSNQTKNFVKVYKSGSVGRSLDISRFSSYHELREELGKMFAIEGLLEDPLRSGWQLVFVDRENDILLLGDDPWESFVNNVWYIKILSPEDVHQMGDHGEG

SEQ ID NO: 94 - protein, 100 aa, Synthetic construct
PTPNNCTFVKVYKSGSVGRSIDISRFSSYHELREELGQMFGIEGKLEYRLRSGWQLVFVDRENDVLLLGDDPWESFVNNVWYIKILSPEDVHKMGHQAVE

SEQ ID NO: 95 - protein, 100 aa, Synthetic construct
QLTPTRTFVKVYKSGSVGRSLDISRFSSYNELREELGQMFGIEGKFEDPLRSGWQLVFVDRENDVLLLGDDPWEAFVSNVWYIKILSPEDVQKMGEQGVE

SEQ ID NO: 96 - protein, 101 aa, Synthetic construct
PSNPTRTFVKVYKTGSVGRSLDISRFSSYQELREELAQMFGIEGQLVEDPRRSGWQLVFVDRENDVLLLGDDPWEAFVNNVGFIKILSPEDFQKLGEQAIE

SEQ ID NO: 97 - protein, 100 aa, Synthetic construct
ANQTNRIFVKVQKSGSFGRSLDISKFSSYHELRSELARMFGLEGLLEDPERSGWQLVIVDRENDVLLLGDDPWQEFVNNVWYIKILSPYEVQQMGKEGLD

SEQ ID NO: 98 - protein, 83 aa, Synthetic construct
TRTFVKVYKSGSVGRSLDISRFSSYHELREELGQMFGIEGLEDPRSGWQLVFVDREDLLLGDDPWFVNNVWIKILSPEVQKGE

SEQ ID NO: 99 - DNA, 40 bp, Synthetic construct
  source 1..40: mol_type = other DNA
gtatctcactgcaagtgataccagtacgcatggcgagttt

SEQ ID NO: 100 - skipped

SEQ ID NO: 101 - DNA, 33 bp, Synthetic construct
  source 1..33: mol_type = other DNA
gtatctcactgcaagtgatagcatggcgagttt

SEQ ID NO: 102 - DNA, 36 bp, Synthetic construct
  source 1..36: mol_type = other DNA
gtatctcactgcaagtgataccagcatggcgagttt

SEQ ID NO: 103 - DNA, 41 bp, Synthetic construct
  source 1..41: mol_type = other DNA
gtatctcactgcaagtgataccagtaccgcatggcgagttt

SEQ ID NO: 104 - DNA, 33 bp, Synthetic construct
  source 1..33: mol_type = other DNA
gtatctcactgcaagtgataccatggcgagttt

SEQ ID NO: 105 - DNA, 36 bp, Synthetic construct
  source 1..36: mol_type = other DNA
gtatctcactgcaagtgataccagtatggcgagttt

SEQ ID NO: 106 - DNA, 32 bp, Synthetic construct
  source 1..32: mol_type = other DNA
ctgactgcaagtgatactagcacgcatggtgg

SEQ ID NO: 107 - DNA, 30 bp, Synthetic construct
  source 1..30: mol_type = other DNA
ctgactgcaagtgatactagcgcatggtgg

SEQ ID NO: 108 - DNA, 33 bp, Synthetic construct
  source 1..33: mol_type = other DNA
ctgactgcaagtgatactagcaccgcatggtgg

SEQ ID NO: 109 - DNA, 30 bp, Synthetic construct
  source 1..30: mol_type = other DNA
ctgactgcaagtgatactaacgcatggtgg

SEQ ID NO: 110 - DNA, 40 bp, Synthetic construct
  source 1..40: mol_type = other DNA
atgcacaaactgtgccccgtacatttgtcaaggttctagt

SEQ ID NO: 111 - DNA, 41 bp, Synthetic construct
  source 1..41: mol_type = other DNA
atgcacaaactgtgccccgtacatttgttcaaggttctagt

SEQ ID NO: 112 - DNA, 39 bp, Synthetic construct
  source 1..39: mol_type = other DNA
atgcacaaactgtgccccgtacattttcaaggttctagt

SEQ ID NO: 113 - DNA, 23 bp, Synthetic construct
  source 1..23: mol_type = other DNA
cagctgtaagggctctctccctt

SEQ ID NO: 114 - DNA, 24 bp, Synthetic construct
  source 1..24: mol_type = other DNA
gattgattctgttggcccctttcc

SEQ ID NO: 115 - DNA, 24 bp, Synthetic construct
  source 1..24: mol_type = other DNA
cccatgtgacatgttcatagccac

SEQ ID NO: 116 - DNA, 23 bp, Synthetic construct
  source 1..23: mol_type = other DNA
gctggtcctggcattgcacatcc

SEQ ID NO: 117 - DNA, 23 bp, Synthetic construct
  source 1..23: mol_type = other DNA
ccagttggcacattccagaaagg

SEQ ID NO: 118 - DNA, 21 bp, Synthetic construct
  source 1..21: mol_type = other DNA
tcccttgtgaggtttgcacac

SEQ ID NO: 119 - DNA, 23 bp, Synthetic construct
  source 1..23: mol_type = other DNA
taccagatcctaccacttcccac

SEQ ID NO: 120 - DNA, 22 bp, Synthetic construct
  source 1..22: mol_type = other DNA
cccccatggccttctccttacc

SEQ ID NO: 121 - DNA, 20 bp, Synthetic construct
  source 1..20: mol_type = other DNA
caaccaacaacctgtgcctt

SEQ ID NO: 122 - DNA, 22 bp, Synthetic construct
  source 1..22: mol_type = other DNA
gccttcccttaggatatgattc

SEQ ID NO: 123 - DNA, 22 bp, Synthetic construct
  source 1..22: mol_type = other DNA
gtaaactccacctctccatccc

SEQ ID NO: 124 - DNA, 23 bp, Synthetic construct
  source 1..23: mol_type = other DNA
ggatgatcactcatttcttggac

SEQ ID NO: 125 - DNA, 23 bp, synthetic construct
  source 1..23: mol_type = other DNA
ctaccttatttgttggcttgaat

SEQ ID NO: 126 - DNA, 21 bp, Synthetic construct
  source 1..21: mol_type = other DNA
gggtgccttgtggtgacagat

SEQ ID NO: 127 - DNA, 24 bp, Synthetic construct
  source 1..24: mol_type = other DNA
ctggatgaagtacaacctcttgag

SEQ ID NO: 128 - DNA, 20 bp, Synthetic construct
  source 1..20: mol_type = other DNA
ggtagtttcatatgacatgc

SEQ ID NO: 129 - DNA, 21 bp, Synthetic construct
  source 1..21: mol_type = other DNA
ccacggttcaaatccaagacg

SEQ ID NO: 130 - DNA, 20 bp, Synthetic construct
  source 1..20: mol_type = other DNA
gtagcatccaactaatgtct

SEQ ID NO: 131 - DNA, 24 bp, Synthetic construct
  source 1..24: mol_type = other DNA
ggacaagtcacgaataacaatccc

SEQ ID NO: 132 - DNA, 22 bp, Synthetic construct
  source 1..22: mol_type = other DNA
gcaagacactcccgtgacaagc

SEQ ID NO: 133 - DNA, 24 bp, Synthetic construct
  source 1..24: mol_type = other DNA
gatctctctttgaatggtgtctcc

SEQ ID NO: 134 - DNA, 25 bp, Synthetic construct
  source 1..25: mol_type = other DNA
aagcgcaatatgatatacaattccg

(19) United States
(12) Patent Application Publication
(10) Pub. No.: US 2024/0417745 A1
(43) Pub. Date: Dec. 19, 2024

(54) SYSTEMS AND METHODS FOR SEEDLESS FRUIT TRAIT

(71) Applicant: Duke University, Durham, NC (US)

(72) Inventors: Tai-Ping Sun, Durham, NC (US); Jianhong Hu, Cary, NC (US)

(73) Assignee: Duke University, Durham, NC (US)

(21) Appl. No.: 18/629,315

(22) Filed: Apr. 8, 2024

Related U.S. Application Data
(60) Provisional application No. 63/457,533, filed on Apr. 6, 2023.

(51) Int. Cl. C12N 15/82; C07K 14/415

(52) U.S. Cl. CPC C12N 15/8262; C07K 14/415; C12N 15/8273

(57) ABSTRACT

The present disclosure provides genetically modified flowering plants that produce seedless fruit and methods for making and cultivating such plants. The genetically modified flowering plants express a modified Auxin Response Factor 8A (ARF8A) or an ortholog thereof.

### PRIOR RELATED APPLICATION

This application claims the benefit of U.S. Provisional Application No. 63/457,533, filed on Apr. 6, 2023, which is hereby incorporated by reference in its entirety.

### STATEMENT AS TO RIGHTS TO INVENTIONS MADE UNDER FEDERALLY SPONSORED RESEARCH AND DEVELOPMENT

This invention was made with government support under Grant No. 2018-67013-27395 awarded by the U.S. Department of Agriculture. The government has certain rights to this invention.

### FIELD

This disclosure describes compositions, cells, plants, and methods for growing plants having a seedless fruit trait.

### REFERENCE TO A SEQUENCE LISTING SUBMITTED AS AN XML FILE

The official copy of the sequence listing is submitted electronically as an .xml formatted sequence listing with a file named 1434269-DU7821US.xml, created on Jul. 9, 2024, and having a size of 127 kb. The sequence listing contained in this .xml formatted document is part of the specification and is herein incorporated by reference in its entirety.

### BACKGROUND

Fruit development is essential for the reproduction of flowering plants. Agronomically, fruits are a crucial food source. The transition from ovary to fruit, also called fruit initiation or fruit set, is arguably the most critical step in fruit development because the ovary is programmed to abort unless ovules are fertilized. Since fruit initiation only occurs following successful fertilization, resources are not wasted on producing seedless fruit (i.e., parthenocarpy), especially under adverse conditions that cause impaired anther/pollen development. Nonetheless, parthenocarpy is a desirable trait for fruit crops and ensures consistent fruit yield in variable environmental conditions. However, developing parthenocarpic fruit, without compromising other desirable characteristics is problematic.

### SUMMARY

Provided herein are genetically modified flowering plants that produce seedless fruit. In some embodiments, the genetically modified flowering plant expresses a modified Auxin Response Factor 8A (ARF8A) or an ortholog thereof, wherein the modified ARF8A or an ortholog thereof comprises a mutation in the Phox and Bem1 (PB1) domain, and wherein the flowering plant produces ovary-derived, seedless fruit. In some embodiments, the mutation is one or more amino acid substitutions in the PB1 domain. In some embodiments, the mutation is a deletion in the PB1 domain. In some embodiments, the entire PB1 domain or a portion thereof is deleted. In some embodiments, the interaction between ARF8A or an ortholog thereof and an auxin responsive protein is reduced in the plant.

In some embodiments, the auxin responsive protein is an Aux/IAA transcription repressor protein. In some embodiments, the modified ARF8A is Solanum lycopersicum ARF8A (SlARF8A) or an ortholog thereof. In some embodiments, the ARF8A is Solanum lycopersicum ARF8A (SlARF8A) and the Aux/IAA transcription repressor protein is Solanum lycopersicum IAA (SlAA9). In some embodiments, the SlARF8A ortholog comprises is Solanum lycopersicum SlARF8B, Arabidopsis thaliana AtARF8, Capsicum annuum CaARF8, Solanum melongena SmARF8, Cucumis sativus CsARF8, Malus domestica MdARF8, or Citrus sinensis CsARF8.

In some embodiments, the modified SlARF8A comprises a deletion in amino acids 716-804 of SEQ ID NO: 1. In some embodiments, amino acids 716-804 of SEQ ID NO: 1 or a portion thereof are deleted. In some embodiments, the modified SlARF8A is a truncated SlARF8 comprising amino acids 1-716 of SEQ ID NO: 1.

In some embodiments, the SlARF8A ortholog is a polypeptide comprising a sequence having at least 90% identity (e.g., at least 90%, 91%, 92%, 93%, 94%, 95%, 96%, 97%, 98%, 99%, or 100% identity) to at least one of SEQ ID NOs: 90-97. In some embodiments, the SlARF8A ortholog is a polypeptide comprising a sequence having at least 95% identity (e.g., at least 95%, 96%, 97%, 98%, 99%, or 100% identity) to SEQ ID NO: 98. In some embodiments, the modified SlARF8A ortholog comprises a deletion in amino acids corresponding to amino acids 716-804 of SEQ ID NO: 1. In some embodiments, the modified SlARF8A ortholog comprises a deletion of amino acids corresponding to amino acids 716-804 of SEQ ID NO: 1 or a portion thereof. In some embodiments, the modified SlARF8A ortholog comprises amino acids corresponding to amino acids 1-716 of SEQ ID NO: 1.

In some embodiments, the plant is a genetically modified plant from the clade Angiospermae. In some embodiments, the plant is a genetically modified plant selected from the group consisting of: a Solanaceae plant, a Cucurbitaceae plant, an Ericaceae plant, a Rutaceae plant, a Vitaceae plant, an Anacardiaceae plant, a Lauraceae plant, a Moraceae plant, a Cactaceae plant, a Caricaceae plant, a Ebenaceae plant, a Myrtaceae plant, a Annonaceae plant, a Rhamnaceae plant, and a Sapindaceae plant. In some embodiments, the plant is a genetically modified Solanaceae plant. In some embodiments, the plant is a genetically modified Solanun plant. In some embodiments, the Solanum plant is a tomato plant or an eggplant.

In some embodiments, the fruit from the plant exhibits placental growth that is at least 80% of the placental growth exhibited by a fruit from a wildtype plant of the same species. In some embodiments, the plant exhibits increased yield as compared to a control. In some embodiments, the plant exhibits increased yield under temperature stress conditions as compared to a control. In some embodiments, the temperature stress is heat stress or cold stress.

In some embodiments, one or both alleles of an arf8a gene or an ortholog thereof comprise a mutation in the genomic sequence encoding the PB1 domain. In some embodiments, the mutation is an insertion, a deletion or substitution of one or more nucleic acids in the genomic sequence encoding the PB1 domain. In some embodiments, the amount and/or activity of ARF8A produced by the plant is decreased. In some embodiments, the amount and/or activity of ARF8A mRNA produced by the plant is decreased. In some embodiments, the plant comprises an expression construct, wherein the expression construct comprises a promoter operably linked to a recombinant nucleic acid sequence encoding ARF8A or an ortholog thereof comprising a mutation in the Phox and Bem1 (PB1) domain.

Also provided is a method of producing any of the genetically modified plants described herein, wherein the method comprises a) modifying one or both alleles of an arf8a gene gene in one or more flowering plant cells to introduce a mutation into the nucleic acid sequence of the PB1 domain; and b) generating one or more flowering plants from the one or more flowering plant cells. In some methods, the genome of the one or more flowering plant cells is modified by contacting the one or more flowering plant cells with an expression construct comprising a nucleic acid sequence encoding ARF8 or an ortholog thereof comprising a mutation in the Phox and Bem1 (PB1) domain. In some methods, the genome of the one or more flowering plant cells is modified by using Clustered Regularly Interspaced Short Palindromic Repeats (CRISPR) gene editing. Some methods further comprising obtaining fruit from the one or more flowering plants. Some methods further comprising crossing the genetically modified flowering plant with a wildtype plant of the same species.

### DESCRIPTION OF THE FIGURES

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1a shows a sequence alignment of the PB1 domain of ARF8 orthologs from tomato (ARF8A and ARF8B; SEQ ID NOs: 90 and 91), pepper (SEQ ID NO: 92), Arabidopsis (SEQ ID NO: 93), apple (SEQ ID NO: 94), orange (SEQ ID NO: 95), cucumber (SEQ ID NO: 96), and eggplant (SEQ ID NO: 97), and a consensus sequence of this alignment (SEQ ID NO: 98). The PB1 domain was formerly designated as domain III/IV. The boundaries of the domain III and domain IV portions are marked by the horizontal bars under the alignment, respectively. The PB1 domain starts with the first amino acid of domain III, and ends with the last amino acid of domain IV. The conserved lysine residue in domain III is highlighted. The conserved OPCA motif residues in domain IV are highlighted. The amino acid numbers (aa #) in each peptide indicate their distance from the first aa of each ARF8 protein.

FIGS. 1b-1l show that arf8a and arf8b crispr knockout mutants displayed strong parthenocarpy, according to certain embodiments of this disclosure. b, Diagram of the SlARF8A/8B protein structure. DBD, DNA binding domain. MR, middle region. PB1, Phox and Bem1 domain. Arrow indicates the target position of CRISPR gRNAs. c-d, List of arf8a and arf8b crispr null alleles, respectively (8A (SEQ ID NO: 99), 8a-2 (SEQ ID NO: 100), 8a-3 (SEQ ID NO: 101), 8a-4 (Seq ID NO: 102), 8a-5 (SEQ ID NO: 103), 8a-6 (SEQ ID NO: 104) in FIG. 1b; 8B (SEQ ID NO: 105), 8b-1 (SEQ ID NO: 106), 8b-2 (SEQ ID NO: 107), 8b-3 (SEQ ID NO: 108), and 8b-4 (SEQ ID NO: 109) in FIG. 1c). Numbers on top of sequences: distances from the ATG start codon of SlARF8 cDNA sequences. Letters in boldface: gRNA 20-mer sequences targeting SlARF8A or SlARF8B. PAM: protospacer adjacent motif. Numbers/letters after each sequence: # of nucleotide deletions/insertions in each crispr allele, and the nucleotide insertion type. Letters highlighted in gray: insertions. Dashes in thin or thick line: deletions or no insertion. e, Schematic of mature WT tomato fruit derived from self-pollinated flower. f-i, Parthenocarpic growth in arf8a-1, arf8b-1 single and double mutants. In f, pictures were taken five weeks after emasculation. Bar=2 cm. In g, parthenocarpy frequency was calculated as % of parthenocarpic fruits developed from emasculated flowers. Means ±SE from two biological replicas. n=29-39 for total number of emasculated flowers per line. j-l, entire failed to rescue 8a 8b placenta growth defect. In j, pictures were taken five weeks after emasculation. Bar=2 cm. Means ±SE from two biological replicas. n=28-51 for total number of emasculated flowers per line. In g-i and k-l, different letters above bars represent significant differences, p<0.05 (g, k, l) or p<0.01 (h, i). The p values were made with one-sided (g, k) or two-sided (h, i, l) analysis. In boxplots h, i and l, center lines and box edges are medians and the lower/upper quartiles, respectively. Whiskers extend to the lowest and highest data points within 1.5× interquartile range (IQR) below and above the lower and upper quartiles, respectively.

FIGS. 2a-f show phenotypes of fruits produced by self-pollinated WT, arf8a, arf8b and 8a 8b mutants according to certain embodiments of this disclosure. a, Fruits of WT, arf8a-1, arf8b-1 and 8a 8b. Bar=2 cm. b, Average fruit diameters. Photo and measurements were taken at 5 weeks after anthesis (from self-pollinated flowers). Results from two biological replicas. n=10-16 for #of fruits per line. c, Photos of representative flower (top) and fruit (bottom) clusters of WT and 8a 8b. Bar=2 cm. The photo of the fruit clusters was taken 6 weeks after anthesis of the first flower in the inflorescence. d, Average frequency of fruit formation (%) per fruit cluster. Results from 8 fruit clusters per line. Flower #per cluster=4-13. Fruit #per cluster=2-9. e, Average fruit yield per fruit cluster (g). Fruit clusters n=8 per line. f, Average single fruit weight (g). n=23-49 for fruit #in 8 clusters per line. In boxplots of b and d-f, center lines and box edges are medians and the lower/upper quartiles, respectively. Whiskers extend within 1.5× IQR. Different letters above bars represent significant differences, p<0.01 in b and f and p<0.05 in d and e. The p values were made with two-sided analysis.

FIGS. 3a-i show that arf and iaa9 mutants were rescued by P_{ARF}:tag-ARF or P_{IAA9}:tag-IAA9 transgenes, respectively, according to certain embodiments of this disclosure. a-b, Parthenocarpy in arf8a-1 was rescued by the P_{ARF8A}:3F2H-ARF8A transgene. Bar=2 cm. Means ±SE from two biological replicas. n=24-29 for total number of emasculated flowers per line. d-e, Parthenocarpy in arf8b-1 was rescued by the P_{ARF8B}:3F2H-ARF8B transgenes. Bar=2 cm. Means ±SE from two biological replicas. n=16-21 for total number of emasculated flowers per line. c, Mature leaves of WT, arf5-1 and P_{ARF5}:3F-ARF5 arf5-1 lines. Bar=5 cm. f, 0 DAA flowers of WT, arf5-1 and 3F-ARF5 arf5-1 lines. Bar=5 mm. g-h, Parthenocarpy in entire was rescued by the P_{IAA9}:3F2H-IAA9 transgene. Bar=2 cm. Means ±SE from two biological replicas. n=22 for total number of emasculated flowers per line. i, Mature leaves of WT, entire and 3F2H-IAA9 entire lines. Bar=5 cm. In a-b, d-e, and g-h, photos and measurements were taken five weeks after emasculation. In b, e and h, different letters above bars represent significant differences, p<0.05. The p values were made with two-sided analysis.

FIGS. 4a-k show additional phenotypes of arf8a-1, arf8b-1 mutants, according to certain embodiments of the disclosure. a, arf8a-1 and arf8b-1 had additive effects on plant height and leaf size. Top: 5-week-old plants. Bottom: the 3rd oldest leaves on main stem. Bar=5 cm. b, Plant height of WT, arf8a-1, arf8b-1 and arf8a-1 8b-1. c, Average #of internodes on main stem. d, length of #3 internode on main stem. e, #of axillary buds on main stem. In b-e, 5-week-old plants were measured. Means ±SE. n=8 for #of plants per line. Different letters above bars represent significant differences, p<0.01, except in e (p<0.05). f, Flower phenotype of arf8a-1 and arf8b-1 single and double mutants. Top: whole flower at 0 DAA. Bottom: style/ovary and anther cone from 0 DAA flower. Bar=5 mm. g-h, Average lengths of style and anther cone in 0 DAA flowers of arf8a-1, arf8b-1 mutants. Means ±SE from 9-10 flowers per line. i-k, Reciprocal cross between WT and arf8a-1 8b-1. Photos and measurements were taken 7-week after crosses. Bar=2 cm. Means ±SE from two biological replicas. n=10-22 for number of each cross. g-h and j-k, different letters above bars represent significant differences, p<0.01, except in j (p<0.05). The p values were made with two-sided analysis.

FIGS. 5a-g show that arf8a-2, arf8b-2 parthenocarpic and whole plant phenotypes resembled 8a-1 8b-1 mutants, according to certain embodiments of the disclosure. a-c, Parthenocarpic growth in arf8a-2, arf8b-2 single and double mutants. Pictures were taken five weeks after emasculation. Bar=2 cm. Parthenocarpy efficiency was calculated by % of parthenocparpic fruits developed from emasculated flowers. In b, Means ±SE from two biological replicas. n=37-62 for total number of emasculated flowers per line. The low parthenocarpy of WT line in this experiment was likely due to seasonal high temperature in the greenhouse. In boxplot c, center lines and box edges are medians and the lower/upper quartiles, respectively. Whiskers extend within 1.5× IQR. d-e, Whole plant and leaf phenotypes of arf8a-2, arf8b-2 and arf8a-2 8b-2. Plants were 5-week-old. Leaves were the 3rd oldest on main stem from the same age plants. Bar=5 cm. Means ±SE. n=8 for number of plants per line. f-g, Average #of internodes on main stem, and the length of #3 internode on main stem in 5-week-old arf8a-2, arf8b-2 mutants. Means ±SE. n=8 for number of plants per line. b-c and e-g, different letters above bars represent significant differences, p<0.01, except in b (p<0.05). The p values were made with one-sided (b) or two-sided (c, e-g) analysis.

FIGS. 6a-f show that ARF5, ARF7, ARF8A and ARF8B all contribute to fruit initiation and growth, according to certain embodiments of the disclosure. a-c, Parthenocarpy phenotypes of various higher order combinations of arf5, 7, 8a, 8b mutants. d-f, Parthenocarpy phenotypes of various combinations of arf5/+, 7, 8a, 8b mutants. Parthenocarpy frequency (b, e) was shown as means ±SE from two biological replicas. In c and f, center lines and box edges are medians and the lower/upper quartiles, respectively. Whiskers extend within 1.5× IQR. n=26-49 (b, c) or 15-28 (e, f) for total number of emasculated flowers per line. Photos and measurements were taken five weeks after emasculation. Bar=2 cm. Different letters above bars represent significant differences, p<0.01 (in c) or p<0.05 (in b, e, f). The p values were made with one-sided (b, e) or two-sided (c, f) analysis.

FIGS. 7a-d show that ARF8A contributes to fruit growth in arf5 7 8b mutant, according to certain embodiments of the disclosure. a, arf5 and arf5 arf7 mutants displayed severe flower development defects. Images were taken using 0 DAA flowers and ovaries. Bar=5 mm. b-d, Parthenocarpy phenotypes of higher order arf mutants. Photos and measurements were taken five weeks after emasculation. Bar=1 cm. c-d, Parthenocarpy frequency (c) and fruit diameters (d). Means ±SE from two biological replicas. n=18-30 for total number of emasculated flowers per line. Different letters above bars represent significant differences, p<0.05. The p values were made with one-sided analysis.

FIG. 8 shows that IAA9 inhibition of ARF8A transactivation activity requires the PB1domain, according to certain embodiments of the disclosure. Tomato auxin response circuit analysis in yeast (ARC^{sc}) showed that ARF8A activation of the auxin responsive promoter was inhibited by the TPL1N100-IAA9 fusion, which had no effect on ARF8A-NT. Means ±SE of 3 biological replicas are shown. *p<0.05. The p values were made with two-sided analysis. Reporter only, ARF8A, and ARF8A-NT are listed from left to right and, for each, data is shown for −IAA9 (left) and +IAA9 (right).

FIGS. 9a-d show the parthenocarpy phenotypes of 3/2H-ARF8A-OE, 3F2H-ARF8A-NT and 3F2H-ARF8B-NT transgenic lines, according to certain embodiments of the disclosure. a-c, Photos and measurements were taken five weeks after emasculation. Bar=2 cm. d, Detection of 3F2H-ARF8A (in both normal and OE lines), 3F2H-8A-NT, 3F2H-ARF8B and 3F2H-8B-NT proteins in-2 DAA ovaries by α-HA antibody. WT was included as a negative control, and α-tubulin (TUB) was included for immunoblotting to show similar loadings. Note that 3F2H-ARF8A-OE, 3F2H-ARF8-NT proteins were extracted from T0 hemizygous ovary while 3F2H-ARF8from T1 homozygous ovary. b-c, Parthenocarpy frequency (b) and fruit diameters (c) of 8A-OE, 8A-NT and 8B-NT lines. b, Means ±SE from two biological replicas. n=30-32 for total number of emasculated flowers per line. In boxplot c, center lines and box edges are medians and the lower/upper quartiles, respectively. Whiskers extend within 1.5× IQR. Different letters above bars represent significant differences, p<0.01 (in b, c). The p values were made with one-sided (b) or two-sided (c) analysis.

FIGS. 10a-i show that ARFs and IAA9 showed distinctive spatial localization in ovary, according to certain embodiments of the disclosure. a, Absolute transcript levels of SlARFs and SlIAA9 in four different tissues (pericarp, ovule, placenta, septum) of −2 DAA ovary. The transcript levels were calculated using standard curves, and are shown as copies of ARFs/IAA9 cDNA per 10² copies of SlUBQ7. Means ±SE of 3 biological replicas are shown. For the graph on the left, for each plant part, data is shown from left to right for ARF5, ARF7, ARF8A, and ARF8B, b-h, ARFs/IAA9 protein localization in −2 DAA ovaries of different transgenic tomato lines as labeled by immunoblot analyses. Pericarp (Pc), Ovule (O), Placenta (Pl) and Septum(S). Transgenic tomato lines expressing epitope-tagged ARFs and IAA9 showed distinctive protein localization in different ovary tissues: (b) 3FLAG-2HA-ARF8A (3F2H-ARF8A); (c) 3F2H-ARF8B; (d) 3F2H-ARF8A-NT; (e) 3F2H-ARF8B-NT; (f) 3F-ARF5; (g) 3F-ARF7; (h) 3F2H-IAA9. Non-transgenic WT −2 DAA whole ovary was used as a negative control. i, Temporal distribution of 3F2H-IAA9 protein in ovaries before and after anthesis. WT −2 DAA ovary was used as a negative control. Arrow: epitope-tagged ARF or IAA9 proteins. *: non-specific proteins. Tagged-ARFs were detected by α-FLAG antibody while tagged-IAA9, ARF8A-NT or ARF8B-NT by α-HA antibody. α-Tubulin (TUB) was included for immunoblotting to show similar loadings.

FIGS. 11a-f show spatial and temporal distribution of SlARFs and SlIAA9 transcripts in ovary according to certain embodiments of the disclosure. Absolute transcript levels of SlARFs and SlIAA9 were measured in different ovary tissues (pericarp, ovule, placenta, septum) from stages around anthesis (−2, 0, +2, +5 DAA) by RT-qPCR. a, SlARF5; b, SlARF7; c, SlARF8A; d, SlARF8B; e, SlIAA9. The transcript levels were calculated using standard curves for SlARFs19 and for SlIAA9 (in f), and are shown as copies of SlARF/IAA9 cDNA per 102 copies of UBQ7. Means ±SE of 3 biological replicas are shown. f. Standard curve for absolute quantification of SlIAA9 transcript level by qPCR. For each bar graph, for each plant part, data is shown from left to right for −2 DAA, 0 DAA, +2 DAA, and +5 DAA.

FIGS. 12a-b show the | Identification of 8a 8b- and auxin-responsive genes in ovary by RNA-seq analysis, according to certain embodiments of the invention. RNA-seq analysis was performed using −2 DAA ovaries of 8a 8b and WT, and −2 DAA WT ovaries ±100 μM 2,4-D for 2, 6 or 24 h. a-b, Venn diagrams of coregulated DEGs by 8a 8b and/or +2,4-D 2/6/24 h treatments. Down-regulated DEGs in a, Upregulated DEGs in b.

FIGS. 13a-i show spatial expression patterns of auxin-responsive ARF8A/8B target genes, according to certain embodiments of the disclosure. a-d, RT-qPCR analysis of WT vs. 8a 8b (a-b) or WT after mock vs. 2,4-D 24 h treatment (c-d) to verify selected down-regulated and up-regulated genes that were identified from RNA-seq data in FIG. 12. RNA from −2 DAA whole ovary of WT and 8a 8b (a, b) or WT after mock vs 2,4-D treatment for 24 h (c, d) were used. e-i, RT-qPCR showing differential expression patterns of SlARF8A/8B target genes (e: AG1; f: MADS2; g: AGL6; h: XTH7; i: GA20ox1). RNA samples from four dissected tissues of-2 DAA ovaries of WT and 8a 8b were used for this analysis. For all RT-qPCR analyses, the housekeeping gene UBQ7 was used to normalize different samples. Means ±SE of 3 biological replicas are shown. a-i, expression level in WT whole ovary was set to 1. Asterisks above bars represent significant differences to their WT counterpart. *p<0.05, **p<0.01. The p values were made with one-sided (b, e-i) or two-sided (a, c, d) analysis.

FIGS. 14a-14c show Confirmation of ARF8A/8B target genes by ChIP-qPCR and RT-qPCR, according to certain embodiments of the disclosure. a, ChIP-qPCR analysis showed SlARF8A binding to promoter regions of AG1, MADS2, AGL6, XTH7 and GA20ox1genes. −2 DAA ovaries of the P_{ARF8A}:3F2H-ARF8A line and anti-FLAG beads were used for the ChIP experiment. Numbers on x-axis represented nt distances (kb) from the start codon of each gene. The relative enrichment was calculated by normalizing against ChIP-qPCR of nontransgenic samples using UBQ7 coding region as control. Means ±SE of 3 biological replicas are shown. b, RT-qPCR analysis showing MADS-BOX genes were downregulated in −2 DAA ovaries of higher order arf mutants, entire and ARF8-NT transgenic lines in comparison to WT, except for MADS2 in 5 7 8a, 5 7 8a 8b/+ and 8A-NT lines. c, RT-qPCR analysis showing upregulation of XTH7 and GA20ox1 in −2 DAA ovaries of arf mutants, entire and ARF8-NT lines. The 8a 8b data in (b) and (c) were the same as in FIGS. 13a and 13b, respectively. For all RT-qPCR analyses, the housekeeping gene UBQ7 was used to normalize different samples. Means ±SE of 3 biological replicas are shown. Expression level in WT whole ovary was set to 1. Asterisks above bars represent significant differences to their WT counterpart. *p<0.05, **p<0.01. The p values were made with one-sided analysis.

FIGS. 15a-d show ARF8A regulates target genes in the absence of IAA9, according to certain embodiments of the disclosure. a-b, RT-qPCR analysis showed that MADS-BOX genes were significantly down-regulated (a) while XTH7 and GA20ox1 genes were up-regulated (b) in +5 DAA WT fruits, comparing to −2 DAA WT ovary. In +2 DAA ovaries, mRNA levels of these ARF8A target genes only showed slight changes or unaltered. For all RT-qPCR analyses, the housekeeping gene UBQ7 was used to normalize different samples. Means ±SE of 3 biological replicas are shown. Expression level in −2 DAA WT ovary was set to 1. c-d, ChIP-qPCR results indicated that ARF8A from +5 DAA fruit (c) and ARF8A-NT from −2 DAA ovary (d) bound to the promoter regions of their target genes AG1, MADS2, AGL6, XTH7 and GA20ox1. Numbers on x-axis represented nt distances (kb) from the start codon of each gene. The relative enrichment was calculated by normalizing against ChIP-qPCR of nontransgenic samples using UBQ7 coding region as control. Means ±SE of 3 biological replicas are shown. Asterisks above bars represent significant differences to their WT counterpart. *p<0.05, **p<0.01. The p values were made with two-sided (a, b) or one-sided (c, d) analysis. In FIG. 15c, for each condition, data is shown in the graph for XTH7 (left) and GA20ox1 (right).

FIGS. 16a-b show a Model of class A-ARFs and IAA9 in controlling fruit initiation and growth in tomato, according to certain embodiments of the disclosure. a, Model of the roles of SlIAA9 and class A-SlARFs (ARF8A, ARF8B, ARF5 and ARF7) in regulating auxin-induced early events in fruit development, divided into two phases. Phase 1: transition from ovary to fruit set. Phase 2: fruit set to fruit growth (including both cell division and cell expansion). The four A-SlARFs display a dual role. Before pollination, SlIAA9/ARF complexes repress fruit set by activating expression of MADS-BOX genes, although the mechanism of this transcription activation is unclear (indicated by a question mark). Upon anthesis, auxin levels are elevated in the ovary, which lead to SlIAA9 degradation and release class A-SlARFs to activate fruit growth (by up-regulating growth-related genes). b, Biphasic fruit growth in response to combinatorial regulation of four class A-SlARFs. The graph summarizes parthenocarpy frequency (% of parthenocarpic fruits, green circles in graph) and fruit diameter (blue circles in graph) from emasculated flowers of WT and single, double, triple and quadruple arf mutants. All data were derived from FIG. 6 after normalization using 8a 8b data that are present in different datasets. The general trend of fruit initiation is shown based on parthenocarpy frequency (solid green line) or inferred from down-regulated expression of MADS-BOX genes (dashed green line). The general trend of fruit growth (blue curve) is plotted based on fruit diameters of different genotypes. b was created with BioRender.com free plan.

FIG. 17a is a diagram of the SlARF8A protein structure. DBD (DNA binding domain). MR (middle region). PB1 (Phox and Bem1 domain), according to certain embodiments of the disclosure. Amino acid numbers are shown for domain boundaries. Arrows indicate the target positions of the CRISPR gRNAs, gRNA1 and gRNA2.

FIG. 17b shows the gRNA1 target site sequence of arf8a-CR mutant alleles found in T0 #2 line (8A (SEQ ID NO: 110), 8a-CR-1 (SEQ ID NO: 111), and 8a-CR-2 (SEQ ID NO: 112), according to certain embodiments of the disclosure. Numbers on top of sequences: nucleotide positions from the ATG start codon of SlARF8A genomic DNA sequences. Letters in boldface: gRNA1 20-mer sequence targeting the 5′ end of SlARF8A PB1 coding sequence. PAM: protospacer adjacent motif. Letters underlined or listed at the end of each sequence: nucleotide inserted (+)/deleted (−) in each allele. Dashes in thin or thick line: deletions or no insertion, respectively. Sequence shaded in gray: intron sequence.

FIGS. 18a-b show arf8a-CR T0 mutant phenotypes, according to certain embodiments of the disclosure. a, Parthenocarpic fruits of arf8a-CR T0 #2-4 lines, comparing to seeded WT fruit. All fruits were 4 weeks old after anthesis. Scale bar: 2 cm. b, 39-day-old plants of WT and arf8a-CR T0 #2.

FIG. 19 shows arf8a-CR-1 homozygous mutant produced parthenocarpic fruits, according to certain embodiments of the disclosure. Fruits of WT and arf8a-CR-1 were 4 weeks after anthesis. Scale bar: 2 cm.

FIGS. 20a-b show that the arf8a-CR T0 #2 mutant had higher fruit yield than WT in normal and heat stress conditions, according to certain embodiments of the disclosure. a, Total harvested fruits at the end of 8-week test. Left image: fruits from normal growth condition. Right image: fruits from heat stress condition. Each group of fruits were collected from two plants per line. Scale bar: 5 cm. b, Total fruit yield (left panel) and average fruit weight (right panel) of WT and arf8a-CR T0 #2 plants under normal or heat stress condition.

### DETAILED DESCRIPTION

The following description recites various aspects and embodiments of the present compositions and methods. No particular embodiment is intended to define the scope of the compositions and methods. Rather, the embodiments merely provide non-limiting examples of various compositions and methods that are at least included within the scope of the disclosed compositions and methods. The description is to be read from the perspective of one of ordinary skill in the art; therefore, information well known to the skilled artisan is not necessarily included. Introduction

Studies on the model plant tomato (Solanum lycopersicum) and other species have shown that plant hormones play pivotal roles in fruit development, regulating every step from initiation to ripening. Auxin is one of the major hormones triggering fruit initiation. After pollination, auxin levels increase in developing seeds, and that is essential to trigger the transition from ovary to fruit development. Auxin also promotes cell division and expansion during fruit growth. In addition, application of auxin to unfertilized ovary or genetic mutations that cause elevated auxin signaling can both induce seedless fruit formation. Thus, understanding the mechanism of auxin-induced parthenocarpy is important for developing climate-resilient crops.

The auxin signaling pathway is well conserved among land plants. The key components of early auxin signaling has three families of proteins: auxin coreceptors TIR1/AFB, AUX/IAA (IAA) transcription repressors, and AUXIN RESPONSE FACTOR (ARF) transcription factors. When the auxin levels are low, IAA interacts with ARF at the target promoters and recruits corepressor TOPLESS (TPL) to repress transcription by preventing ARF-Mediator complex formation.

When auxin levels increase, auxin binds to both TIR1/AFB and IAA to trigger IAA protein degradation, which releases ARFs to activate the auxin signaling pathway. This canonical AFB/IAA/ARF cascade is based mainly on class A-ARFs (also called activator ARFs). Recent studies have identified several auxin signaling components, all of which inhibit fruit initiation. Among the 25 members of SlIAAs, SlIAA9 is the major repressor for fruit initiation because the tomato SlIAA9 null mutant entire and antisense/CRISPR lines all showed strong parthenocarpy. As for class A-ARFs, silencing of SlARF5 by an artificial miRNA or SlARF7 by RNAi led to strong parthenocarpy, although expression of several class A-ARFs, including SlARF5, SlARF7 and SlARF8B was reduced in the SlARF7 RNAi line. Moreover, slarf5 and slarf7 single mutants do not display parthenocarpy, suggesting that SlARF8A/8B may also inhibit tomato fruit initiation. In Arabidopsis, null alleles of AtARF8 showed parthenocarpy phenotype, indicating AtARF8 represses fruit set. The SmARF8 in eggplant (Solanum melongena) also inhibits fruit initiation as SmARF8 RNAi line presents strong parthenocarpy. In contrast, strawberry (Fragaria vesca) fvarf8 mutants produce larger fruits upon fertilization, but do not display parthenocarpy without pollination, suggesting FvARF8 acts as a major repressor for fruit growth, but not for fruit set. These studies on the class A-ARFs all point to their inhibitory role in fruit initiation/growth. However, these findings are puzzling because auxin is known to play a key role in promoting fruit initiation and because class A-ARFs are in general considered to be essential for activating auxin signaling.

As described in the Examples, several higher order mutant combinations of four class A-SlARFs (SlARF5, SlARF7, SlARF8A, SlARF8B) that are expressed at higher levels in tomato ovaries around anthesis were studied. Surprisingly, all four class A-SlARFs function as inhibitors (together with SlIAA9) in fruit initiation, but as activators in subsequent fruit growth. The parthenocarpic fruit sizes followed a biphasic bell-shaped curve in response to varying arf mutant combinations, revealing the fine-tuning capacity of fruit growth achieved by these four A-ARFs. The lack of placenta growth in the slarf8A slarf8b double mutant further indicated that SlARF8A and SlARF8B are essential for placenta growth. Moreover, the four ARF proteins showed differential spatial localization in the tomato ovary, which is consistent with their mutant phenotypes. RNA-seq and ChIP-qPCR analyses identified three SlARF8A/8B target genes encoding MADS-BOX transcription factors that are key repressors of fruit set, suggesting IAA9/ARFs directly regulates transcription of these MADS-BOX genes. Together, this work demonstrated the four class A-ARFs function in a tissue-specific manner to modulate tomato fruit development by repressing fruit initiation and activating fruit growth. The present disclosure also shows that the PB1 domain of SlARF8A plays a key role in regulating fruit initiation and fruit growth in flowering plants, (e.g., tomato), and can be modified to produce parthenocarpic fruit while minimizing undesirable effects, e.g., placental growth defects, and/or susceptibility to temperature stress.

### Terminology

Unless defined otherwise, all technical and scientific terms used herein have the same meaning as commonly understood by one of ordinary skill in the art to which this disclosure belongs. All patents, patent applications and publications referred to throughout the disclosure herein are incorporated by reference in their entirety.

Articles “a” and “an” are used herein to refer to one or to more than one (i.e. at least one) of the grammatical object of the article. By way of example, “an element” means at least one element and can include more than one element.

The use of any and all examples or exemplary language (e.g., “such as”) provided herein, is intended merely to better illustrate the invention and does not pose a limitation on the scope of the invention unless otherwise claimed.

The terms “may,” “may be,” “can,” and “can be,” and related terms are intended to convey that the subject matter involved is optional (that is, the subject matter is present in some examples and is not present in other examples), not a reference to a capability of the subject matter or to a probability, unless the context clearly indicates otherwise. “About” is used to provide flexibility to a numerical range endpoint by providing that a given value may be “slightly above” or “slightly below” the endpoint without affecting the desired result.

The terms “optional” and “optionally” mean that the subsequently described event, circumstance, or material may or may not occur or be present, and that the description includes instances where the event, circumstance, or material occurs or is present as well as instances where it does not occur or is not present.

The use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially of and “consisting of those certain elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.”

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

### Genetically Modified Plants

Provided in this disclosure are various genetically modified flowering plants that produce seedless (i.e., parthenocarpic) fruit. In some embodiments, the genetically modified flowering plant expresses a modified Auxin Response Factor 8A (ARF8A) or an ortholog thereof, wherein the modified ARF8A or an ortholog thereof comprises a mutation in the Phox and Bem1 (PB1) domain, wherein the flowering plant is a flowering plant that produces ovary-derived seedless fruit.

As used throughout, a plant includes whole plants, derivatives or portions thereof including, shoot vegetative organs/structures (e.g. leaves, stems and tubers), roots, flowers and floral organs/structures (e.g. bracts, sepals, petals, stamens, carpels, anthers and ovules), seed (including embryo, endosperm, and seed coat) and fruit (the mature ovary), plant tissue (e.g. vascular tissue, ground tissue, and the like) and cells (e.g. guard cells, egg cells, trichomes and the like), and progeny of same.

The genetically modified flowering plant can be any flowering plant that produces an ovary-derived fruit. For example, the flowering plant can be a plant from the clade Angiospermae In some embodiments, the Angiospermae plant is selected from the group consisting of a Solanaceae plant, a Cucurbitaceae plant, an Ericaceae plant, a Rosaceae plant, a Rutaceae plant, a Vitaceae plant, an Anacardiaceae plant, a Lauraceae plant, a Moraceae plant, a Cactaceae plant, a Caricaceae plant, Ebenaceae, Aracaceae, Myrtaceae, Annonaceae, Rhamnaceae, a Sapindaceae plant, and a Eudicotidae plant. In some embodiments, the Solanaceae plant is a Solanum (including tomato plant or eggplant) or Capsicum plant (including peppers). In some embodiments, the Cucurbitaceae plant is a Cucurbita plant (including zucchini, squash, pumpkin, gourds (e.g., okra, luffa), a Cucumis plant (including cucumbers, honeydew, cantaloupe, and other melons), or a Citrullus plant (including watermelons). In some embodiments, the Ericaceae plant is a Vaccinium plant (including blueberry, cranberry, and huckleberry). In some embodiments, the Rosaceae plant is from the genus Malus (including apples), Prunus (including plums, cherries, apricots, and peaches), Pyrus (including pears), Rubus (including blackberries and raspberries, or Eriobotrya (including loquat). In some embodiments, Rutaceae plant is a Citrus plant (including oranges, grapefruits, lemons, limes, tangerines, mandarins, and clementines). In some embodiments, the Vitaceae plant is a Vitis plant, including grapes. In some embodiments, the Anacardiaceae plant is a Mangifera plant, including mangos. In some embodiments, the Lauraceae plant is a Persea plant, including avocado. In some embodiments the Moraceae plant is a Ficus plant (including figs) or a Morus plant (including mulberries). In some embodiments, the Cactaceae plant is a Selenicereus plant (including dragon fruits). In some embodiments, the Caricaceae plant is a Carica plant (including papaya). In some embodiments, the Ebenaceae plant is a Diospyros plant (including persimmon). In some embodiments, the Arecaceae plant is a Phoenix plant (including date palms) or a Euterpe plant (including acai berries). In some embodiments, the Myrtaceae plant is a Psidium plant (including guavas) or a Feijoa plant (including pineapple guava). In some embodiments, the Annonaceae plant is an Annona plant (including cherimoya) or an Asimina plant (including pawpaw). In some embodiments, the Rhamnaceae is a Ziziphus plant (including jujubes). In some embodiments, the Sapindaceae plant is a Litchii plant (including lychee), a Dimorcarpus plant (including longan) or a Nephelium plant (including rambutan).

Also provided are plant cells and fruit from any of the flowering plants described herein as well as progeny plants produced from any of the flowering plants described herein, or fruit produced by the flowering plants.

The wildtype ARF8A or an ortholog thereof comprises a PB1 domain that interacts with one or more auxin responsive proteins, in particular Aux/IAA transcription repressor proteins, which repress auxin signaling. Aux/IAA proteins also have PB1 domains, and the interaction between ARF8A or an orthlog thereof and Aux/IAA proteins is mediated through protein-protein interaction of their PB1 domains, which is also referred to as domains III/IV. The PB1 domain interaction is achieved by electrostatic interactions and hydrogen bonding between positive- and negative-charged faces containing conserved positively charged lysine residue and negatively charged OPCA motif, respectively. Therefore, in some embodiments, modification of the PB1 domain of ARF8A or an ortholog thereof, results in a disruption or a decrease in the interaction between the ARF8A or an ortholog thereof, and an auxin responsive protein such as, for example, an Aux/IAA transcription repressor protein. This decrease in the interaction between the proteins does not need to be complete to achieve the desired phenotype. For example, the decrease can be a decrease of at least 20%, 30%, 40%, 50%, 60%, 70%, 80%, 90%, or 100% as compared to the interaction between a wildtype ARF8A or an ortholog thereof and an auxin responsive protein such as, for example, an Aux/IAA transcription repressor protein. In some embodiments, the interaction between the PB1 domain of the ARF8A or an ortholog thereof and an Aux/IAA transcription repressor protein is completely ablated.

Various sequences for ARF8A and orthologs thereof are provided in Table 1. In some embodiments, the ARF8A is Solanum lycopersicum ARF8A (SlARF8A) or an ortholog thereof. The amino acid sequence for SlARF8A is set forth below as SEQ ID NO:1. The PB1 domain, i.e., amino acids 716 to 804 of SEQ ID NO: 1, is underlined in Table 1. As noted above, the PB1 domain is also referred to as the III/IV domain. The domain III portion the PB1 domain is amino acids 716-752 of SEQ ID NO: 1. The domain IV portion of the PB1 domain, i.e., amino acids 765to 804 of SEQ ID NO: 1, is also in boldface in Table 1. A nucleic acid sequence encoding SEQ ID NO: 1 is set forth below as SEQ ID NO: 2. The PB1 domain is underlined in Table 1 and domain IV is boldface.

TABLE 1
ARF8 Sequences

SlARF8A amino acid sequence - SEQ ID NO: 1
MKLSTSGMGQQAHEGENKCLNSELWHACAGPLVCLPTVGSRVVYFPQGHSEQVAATTNKEV
DIHIPNYPNLPPQLICQLHNVTMHADVETDEVYAQMTLQPLTLQEQKDTYLPVELGIPSRQPTN
YFCKTLTASDTSTHGGFSVPRRAAEKVFPPLDFSQTPPCQELIARDLHDIEWKFRHIFRGQPKR
HLLTTGWSVFVSAKRLVAGDSVLFIWNEKNQLFLGIRRATRPQTVMPSSVLSSDSMHIGLLAA
AAHAASTNSCFIVFFNPRASPSEFVIPLSKYIKAVYHTRVSVGMRFRMLFETEESSVRRYMGTIT
GIGDLDPVRWANSHWRSVKVGWDESTAGERQPRVSLWEIEPLTTFPMYPSLFPLRLKRPWYP
GTSSFQENNSEAINGMTWLRGESSEQGPHLLNLQSFGGMFPWMQQRVDPTMLRNDLNQQYQ
AMLASGLQNFGSGDLMKQQLMQFPQPVQYVQHAGSVNPQLQQQQQQQETMQQTIHHHMLP
AQTQDNLQRQQQQHVSNQTEEQSHQHSYQDAYQIPNSQLQQKQPSNVPSPSFSKPDIADPSSK
FSASIAPSGMPTALGSLCSEGTTNFLNFNIIGQQPVIMEQQQQQKSWMAKFANSQLNMGSSSPS
LSGYGKETSNSQETCSLDAQNQSLFGANVDSSGLLLPTTVSNVATTSIDADISSMPLGTSGFPNP
LYSYVQDSTDLLHNVGQADAQTVPRTFVKVYKSASLGRSLDITRFNSYHELRQELGQMFGIEG
FLENPQRSGWQLVFVDRENDVLLLGDDPWEEFVNNVWYIKILSPEDVQKLGKEEVGSLNR
GPPERMSSNNSADGRDFMSGLPSIGSLDY
SlARF8A nucleotide sequence - SEQ ID NO: 2
ATGAAGCTTTCAACATCAGGAATGGGTCAGCAAGCTCATGAAGGAGAGAACAAGTGTTTG
AATTCAGAACTATGGCATGCTTGTGCTGGTCCCCTTGTTTGTCTACCAACGGTAGGGAGTC
GAGTGGTTTACTTTCCTCAGGGTCACAGTGAACAGGTTGCGGCGACAACTAATAAAGAAG
TCGATATTCACATACCTAATTACCCGAACTTGCCACCACAGTTGATCTGTCAACTCCACAA
TGTCACAATGCATGCAGATGTTGAAACGGATGAAGTATATGCTCAGATGACATTGCAACC
CTTGACACTGCAAGAACAAAAGGACACGTATCTTCCTGTTGAATTGGGTATTCCTAGCAG
GCAGCCTACTAATTATTTTTGCAAGACACTCACTGCAAGTGATACCAGTACGCATGGCGGC
TTTTCTGTTCCTCGTCGTGCTGCAGAGAAAGTTTTCCCTCCTTTGGATTTCTCACAGACACC
ACCCTGTCAAGAATTAATTGCGAGGGATCTGCATGACATCGAATGGAAATTCAGGCATAT
TTTCCGAGGACAGCCTAAGCGGCATCTTCTGACGACTGGCTGGAGTGTGTTTGTTAGTGCT
AAGAGACTTGTTGCCGGAGATTCAGTTCTTTTCATCTGGAATGAGAAAAATCAGCTTTTTT
TGGGAATTCGTCGTGCAACTCGACCTCAGACTGTGATGCCATCATCTGTTCTGTCTAGCGA
CAGCATGCACATTGGATTACTTGCTGCTGCTGCTCATGCTGCCTCTACCAATAGCTGTTTC
ATTGTTTTCTTTAACCCAAGGGCTAGCCCATCCGAGTTTGTTATACCACTTTCAAAATACAT
CAAAGCTGTGTATCACACACGTGTTTCTGTTGGAATGCGTTTCCGGATGCTATTTGAGACT
GAAGAATCAAGTGTTCGAAGGTACATGGGCACAATTACTGGCATTGGTGACTTAGATCCA
GTTCGCTGGGCCAACTCTCACTGGCGGTCTGTCAAGGTTGGTTGGGATGAGTCAACGGCA
GGCGAGAGGCAACCTAGGGTTTCACTATGGGAGATAGAGCCTTTGACTACTTTTCCAATGT
ATCCATCTTTGTTCCCTCTTAGGCTAAAGCGGCCTTGGTATCCAGGAACTTCATCTTTTCAA
GAAAATAACAGCGAAGCTATTAATGGAATGACATGGTTGAGAGGGGAAAGTAGTGAGCA
AGGACCACATCTACTGAATCTTCAATCTTTTGGTGGCATGTTCCCCTGGATGCAACAAAGA
GTTGATCCAACAATGCTCCGAAATGATCTTAACCAGCAGTATCAAGCTATGCTGGCTAGC
GGTTTGCAAAATTTTGGGAGCGGAGATCTGATGAAACAACAACTGATGCAGTTTCCACAG
CCCGTCCAATATGTTCAGCATGCAGGCAGTGTTAATCCTCAACTGCAGCAGCAGCAACAA
CAACAAGAAACAATGCAGCAGACAATTCATCACCATATGTTGCCTGCACAAACTCAAGAT
AACCTTCAAAGGCAACAACAGCAACACGTTAGCAATCAGACAGAGGAGCAATCTCATCA
ACATTCTTACCAGGATGCGTACCAAATACCAAACAGCCAGCTCCAGCAGAAGCAACCATC
AAATGTTCCTTCTCCATCATTTTCAAAGCCAGATATAGCAGATCCAAGCTCCAAGTTCTCG
GCATCCATTGCTCCATCAGGCATGCCAACAGCGCTGGGTTCTTTATGTTCGGAAGGAACTA
CTAACTTTTTGAATTTCAATATAATTGGTCAGCAGCCTGTGATCATGGAGCAGCAGCAGCA
GCAGAAATCTTGGATGGCAAAATTCGCAAATTCACAATTGAACATGGGCTCCAGTTCACC
CTCTCTCTCTGGATATGGGAAAGAAACTTCCAATTCACAGGAAACATGTAGTCTAGATGCC
CAGAATCAATCTCTTTTTGGTGCTAATGTTGATTCTTCAGGGCTTCTCCTCCCTACAACTGT
GTCTAACGTCGCTACTACATCAATTGATGCTGATATATCCTCTATGCCACTAGGGACTTCT
GGATTTCCGAATCCCTTGTATAGTTATGTGCAAGATTCTACTGACTTGTTGCATAATGTAG
GGCAAGCTGATGCACAAACTGTGCCCCGTACATTTGTCAAGGTTTACAAATCAGCGTCCCT
TGGGAGGTCATTGGACATCACTCGGTTCAATAGCTATCATGAGCTACGACAGGAACTTGG
ACAGATGTTCGGGATCGAAGGGTTTCTTGAAAACCCTCAAAGATCAGGCTGGCAGCTTGT
ATTTGTTGACAGGGAGAATGATGTCCTTCTCCTTGGAGACGATCCGTGGGAGGAATTTGTC
AATAATGTTTGGTACATCAAAATTCTTTCACCCGAGGATGTGCAGAAACTGGGGAAAGAG
GAGGTTGGATCCCTAAACCGCGGTCCACCTGAAAGGATGAGCAGTAATAATAGTGCTGAT
GGTCGAGATTTCATGTCCGGACTTCCATCTATAGGATCACTTGATTACTGA
SlARF8B amino acid sequence - SEQ ID NO: 83
MKLSTSGMGQQAHEGGEKKCLNSELWHACAGPLVCLPTVGSRVVYFPQGHSEQVAATTNKE
VDAHIPNYPNLSPQLICQLHNVTMHADVETDEVYAQMTLQPLTPEEQKDTYLPVEFGIPSKQP
TNYFCKTLTASDTSTHGGFSVPRRAAEKVFPPLDFSQTPPAQELIARDLHDVEWKFRHIFRGQP
KRHLLTTGWSVFVSAKRLVAGDSVLFIWNEKNQLLLGIRRAVRPQTVMPSSVLSSDSMHIGLL
AAAAHAAATNSCFNVFFNPRASPSEFVIPLSKYIKAVYHTRVSVGMRFRMLFETEESSVRRYM
GTITGIGDLDPVRWANSHWRSVKVGWDESTAGERQPRVSLWEIEPLTTFPMYPSLFPLRLKRP
FYQGTSSYQDSNNEAINRMSWLRGNAGELGHHSMNLQSFGMLPWMQQRVDSTILPNDINQH
YQAMLATGLQSFGSGDLLKQQLMQFQQPVQYLQHASTENSILHQQQQQQQQIMQQAVHQH
MLPAQTQMLSENLQRQSQHQSNNQSEEQAHQHTYQEAFQLPHDQLQQRQPSNVTSPFLKADF
ADLTSKFSASVAPSGVQNMLGSLCSEGSNNSLNINRTGQSVIIEQSPQQSWMSKFTESQLNTCS
NSSSLPTYGKDTSNPRGNCSLDSQNQALFGANIDSSGHLLPTTVSNVTTTCADMSLMPLGASG
YQNSLYGYVQDSSELLHNAGQIDPPNATHTFVKVYKSGCVGRSLDITQFHSYHELRRELGQMF
GIEGFLEDPQRSGWQLVFVDRENDILLLGDDPWEAFVNNVWYIKILSPEDVQKLGKEEAES
LNRGAVERMSSTNADDRDLISGMPSLGSLEY
AtARF8A amino acid sequence - SEQ ID NO: 84
MKLSTSGLGQQGHEGEKCLNSELWHACAGPLVSLPSSGSRVVYFPQGHSEQVAATTNKEVDG
HIPNYPSLPPQLICQLHNVTMHADVETDEVYAQMTLQPLTPEEQKETFVPIELGIPSKQPSNYFC
KTLTASDTSTHGGFSVPRRAAEKVFPPLDYTLQPPAQELIARDLHDVEWKFRHIFRGQPKRHLL
TTGWSVFVSAKRLVAGDSVIFIRNEKNQLFLGIRHATRPQTIVPSSVLSSDSMHIGLLAAAAHA
SATNSCFTVFFHPRASQSEFVIQLSKYIKAVFHTRISVGMRFRMLFETEESSVRRYMGTITGISD
LDSVRWPNSHWRSVKVGWDESTAGERQPRVSLWEIEPLTTFPMYPSLFPLRLKRPWHAGTSS
LPDGRGDLGSGLTWLRGGGGEQQGLLPLNYPSVGLFPWMQQRLDLSQMGTDNNQQYQAML
AAGLQNIGGGDPLRQQFVQLQEPHHQYLQQSASHNSDLMLQQQQQQQASRHLMHAQTQIMS
ENLPQQNMRQEVSNQPAGQQQQLQQPDQNAYLNAFKMQNGHLQQWQQQSEMPSPSFMKSD
FTDSSNKFATTASPASGDGNLLNFSITGQSVLPEQLTTEGWSPKASNTFSEPLSLPQAYPGKSLA
LEPGNPQNPSLFGVDPDSGLFLPSTVPRFASSSGDAEASPMSLTDSGFQNSLYSCMQDTTHELL
HGAGQINSSNQTKNFVKVYKSGSVGRSLDISRFSSYHELREELGKMFAIEGLLEDPLRSGWQL
VFVDKENDILLLGDDPWESFVNNVWYIKILSPEDVHQMGDHGEGSGGLFPQNPTHL
CaARF8A amino acid sequence - SEQ ID NO: 85
MVPVWMWKEGQPKRHLLTTGWSVFVSAKRLVAGDSVLFIWNDKNQLLLGIRRAIRPQTVMP
SSVLSSDSMHIGLLAAAAHAAATNSCFTVFFNPRASPSEFVIPLSKYIKAVYHTRVSVGMRFRM
LFETEESSVRRYMGTITGIGDLDPVRWANSHWRSVKVGWDESTAGERQPRVSLWEIEPLTTFP
MYPSLFPLRLKRPLYPGTSSYQDSNNEAINRMTWLRGNTSELGPHSMNLQSFGMLPWMQQRV
DSRIIPNDINQHYQAMLATGLQSFGSGDLLKQQLVQFQQPAQYLQHASTDNSILHQQQIMLQA
VHQHMQPAQTQMLSENLQRQSQQQSNNQLEEQAHQHAYQEAFQIPHNQLQQRPSSNVSSQFL
KADFADLNSKFSASVAPSGVQNMLGSLCSEGSSNSLNINRTGQCVIMEQPPQQSWMSKFAESQ
LNTCSNSSPLPTYEKDTSNPRENCSLDSQNQVLFVANVDSSGHLLPTRVSNVTTTCADMSAMP
LGASGYQNSLYGYAQDSSELLHNAGQIDPPNATHTFVKVYKSGCVGRSLDITQFHSYHELRRE
LGQMFGIEGFLEDPQRSGWQLVFVDRENDILLLGDDPWEAFVNNVWYIKILSPEDVQKLG
KEEAESLNRGAVERMSSSSVDDRDLISGMPSLGSLEY
SmARF8A amino acid sequence - SEQ ID NO: 86
MRVSSSGFNPQPEEATGEKKCLNSELWHACAGPLVSLPPVGSRVVYFPQGHSEQVAASTNKE
VDAHIPNYPGLPPQLICQLHNLTMHADVETDEVYAQMTLQPLSPQEQKDVCLLPAELGIPSKQ
PTNYFCKTLTASGTSTHGGFSVPRRAAEKVFPPLDYSQQPPCQELIAKDLHGNEWKFRHIFRGQ
PKRHLLTTGWSVFVSAKRLVAGDAVIFIWNENNQLLLGIRRANRPQTVMPSSVLSSDSMHIGL
LAAAAHAAATNSRFTIFYNPRASPSEFVIPLAKYVKAVYHTRISVGMRFRMLFETEESSVRRY
MGTITGISDLDPVRWPNSHWRSVKVGWDESTAGDRQPRVSLWEIEPLTTFPMYPSPFSLRLKR
PWPSGLPSLTGFPNGDMAMNSPLSWLRGDMGDQGMQSLNFQGFGVTPFMQPRMDASLLGLQ
PDILQTMAALDPSKLANQSLMQFQQSIPNSSASLSQSQMLQPSHSHQNLIQGFSENHLISQAQM
LQQQLQRRQNFNDQQQLLQPQLQQHQEVNSQFQHQQRTKAISSLSQMASVTQPHLSHLPVLS
STGSQQTFSDMLGTHVNSSSNSNMQSLLSSFSRDGAPAVLNMHETHPLVSSSSSSKRIALESQL
PSRVTPFVLSQPENVIAPNTKVSDLSSLLPPFPGRESFSDYKGAEDSQSNALYGFTDSLNILQTG
MSNMKGSSGDNGSLSIPYAISTFTSTVGNEYPLNSDMTASSCVDESGFLQSSENGDQANQTNRI
FVKVQKSGSFGRSLDISKFSSYHELRSELARMFGLEGLLEDPERSGWQLVIVDRENDVLLLGD
DPWQEFVNNVWYIKILSPYEVQQMGKEGLDLLNGVRTQRLPGNVNGCDDYMNQKGSRNT
MNGIPLGSLDY
CsARF8A amino acid sequence - SEQ ID NO: 87
MKLSTSGFGQQDHEGGEKKCLNSELWHACAGPLVSLPTAGTRVVYFPQGHSEQVAATTNKE
VDGHIPNYPNLPPQLICQLHNVTMHADVETDEVYAQMTLQPLTAQEQKDTFLPMELGIPSRQP
TNYFCKTLTASDTSTHGGFSVPRRAAEKVFPPLDFSQQPPAQELIAKDLHDIEWKFRHIFRGQP
KRHLLTTGWSVFVSAKRLVAGDSVLFIWNEKNQLLLGIRRATRPQTVMPSSVLSSDSMHIGLL
AAAAHAAATNSCFTVFYNPRASPSEFVIPLTKYVKAVFHTRVSVGMRFRMLFETEESSVRRYM
GTITGISDLDPVRWPNSHWRSVKVGWDESTAGERQPRVSLWEIEPLTTFPMYPSLFPLRLKRP
WHPGVSSVHDNREDASNGLMWLRGGVGEQGLHSLNLQSVSSLPWLQQRLDSSMFGNDHNQ
QYQAMLAAGMPNLGGVDMLRQQIMHLQQPFQYIPQAGFHNSLLQMQQQQQQQQQQQQQQ
LVQHSMPQNILQAPSQVMAENLPQHILQQTLQNQPEDLPNQQQHTYHDTIQVQSNQFHQGGH
SNVPSPTFPRTDLMDSNTSYSESITSRRNILASSCAEGTGNLSTIYRSGQSILTEHLPQQSPVSKN
AHSQVDAHPNSMSFPPFSGRDSILELRNCNSDSPSPTLFGVNIDSSGLLLPSNVPTYTSPSIGPDS
SSMPLGDSGFQNSLYSCVQDSSELLHNSGQVDPSNPTRTFVKVYKTGSVGRSLDISRFSSYQEL
REELAQMFGIEGQLVEDPRRSGWQLVFVDRENDVLLLGDDPWEAFVNNVGFIKILSPEDFQ
KLGEQAIESFNPIVGQRLTSGGNEAGNVSGLPSVGSLEY
MdARF8A amino acid sequence - SEQ ID NO: 88
MKLSTSGLGQQDHEGGGGGGVEKKCLNSELWHACAGPLVSLPTPGTRVVYFPQGHSDQVAA
TTNKQVDAHIPNYPNLPPQLICQLHNVTMHADVETDEVYAQMTLQPLTPQEQKETFLPMELG
LPSKQPTNYFCKTLTASDTSTHGGFSVPRRAAEKVFPPLDFTLQPPAQELIARDLHDVEWKFRH
IFRGQPKRHLLTTGWSVFVSAKRLVAGDSVLFIWNEKNQLFLGIRRATRPQTVMPSSVLSSDS
MHIGLLAAAAHAASTNSCFTVFYNPRASPSEFVIPLSKYIKAVFHTRVSVGMRFRMLFETEESS
VRRYMGTITGISDLDPVRWPNSHWRSVKVGWDESTSGERQPRVSLWEIEPLTTFPMYPSLFPL
RLKRPWIPGASSMHDNRDEANSLMWLRGGAGEQGLQSMNFQNVGMFPWMQQRLDSTLIGN
DHNQQYQSMLAAGLQNLGSGDQLRQQMMHFQQPFQYVQQSGSHNPMLQLQQQAIQQSIPH
NILQGQPQVSMENMHQQLLQQQFNNQTDAQVQQQQNAYQDALKVQNEQLHQSHVPSPSFTK
PDFSDSSTNFSTSTPRQNVLGTLCPEGRGNLLSMSSAGHSMPTEQSPQQSWDPKYAHGQANAF
SNSMSFPPFNGKDNAVEQENLNSDTHNPTLFGVNIESSGLLFPNTVPSFATSSNDADISMPLGN
TGFQSSLYGCMQDSSELLHGAGQVDPPTPNNCTFVKVYKSGSVGRSIDISRFSSYHELREELGQ
MFGIEGKLEYRLRSGWQLVFVDREDDVLLLGDDPWESFVNNVWYIKILSPEDVHKMGHQ
AVESFSPNTGQRLNSGGGEGQDIVSGLPSLGSLEY
CsARF8A amino acid sequence - SEQ ID NO: 89
MKLSTSGLCQQGHEGDNKCLNSELWHACAGPLVSLPTVGTRVVYFPQGHSEQVAATTNKEV
DSHIPNYPNLPPQLICQLHNVTMHADVETDEVYAQMTLQPLSPEEQKDTFVPIELGIPSKQPTN
YFCKTLTASDTSTHGGFSVPRRAAEKVFPSLDFSLQPPAQELIARDLHDVEWKFRHIFRGQPKR
HLLTTGWSVFVSAKRLVAGDSVLFIWNEKNQLLLGIRRAIRPPTVMPSSVLSSDSMHIGLLAAA
AHAAATNSCFTVFFNPRASPSEFVIPLTKYVKAVFHTRVSVGMRFRMLFETEESSVRRYMGTIT
GISDLDPVRWSNSHWRSVKVGWDESTAGERQPRVSLWEIEPLTTFPMYPSLFPLRLKRPWHPS
TSSFNDNRDETASGLNWLRGGTGEQGLTTLNFQSLGMFPWMQQRVEPSFLGNDHNQQYQAM
LAAGMQSGDPVRQQFMQLQQPFQYLQQSGSQNPLQLKQQQHLLQQLNSQAEDRAQQQQQP
QQHMYHDALQIRTDELLQRQQSNLPSPSFSKANFMDSSTEISVSISPMQNMLGSLPEGSGNLLN
FSGAGPSMLRQQFPQQSLGSKYEPSQVRDFVHSMSLPSSYNGKDAAVGTENCNTDSQNSVVF
GVHIDSSGLLLPTTVSSFTTSVDPGVSSMPLGDSGFHNSMYGCMQDSSELLHNVGQIDQLTPTR
TFVKVYKSGSVGRSLDISRFSSYNELREELGQMFGIEGKFEDPLRSGWQLVFVDRENDVLLL
GDDPWEAFVSNVWYIKILSPEDVQKMGEQGVESFSPSSGQRANSRGNCGRDPVGSLEY

As used herein, the term “ortholog” refers to genes which evolved from a common ancestral gene by speciation. Orthologs of Solanum lycopersicum ARF8A include Solanum lycopersicum SlARF8B (PB1 domain aa. 715-803 of SEQ ID NO: 83), Arabidopsis thaliana AtARF8 (PB1 domain aa. 704-792 of SEQ ID NO: 84), Capsicum annuum CaARF8 (PB1 domain aa. 534-622 of SEQ ID NO: 85), Solanum melongena SmARF8 (PB1 domain aa. 756-844 of SEQ ID NO: 86), Cucumis sativus CsARF8 (PB1 domain aa. 727-816 of SEQ ID NO: 87), Malus domestica MdARF8 (PB1 domain aa. 716-804 of SEQ IN NO: 88), and Citrus sinensis CsARF8 (PB1 domain aa. 690-778 of SEQ ID NO: 89), which are shown in Table 1. The PB1 domain of each is underlined in Table 1, and the domain IV portion is also in boldface. In some embodiments, the SlARF8A ortholog is a polypeptide comprising a sequence having at least 90% identity to at least one of SEQ ID NOs: 83-89.

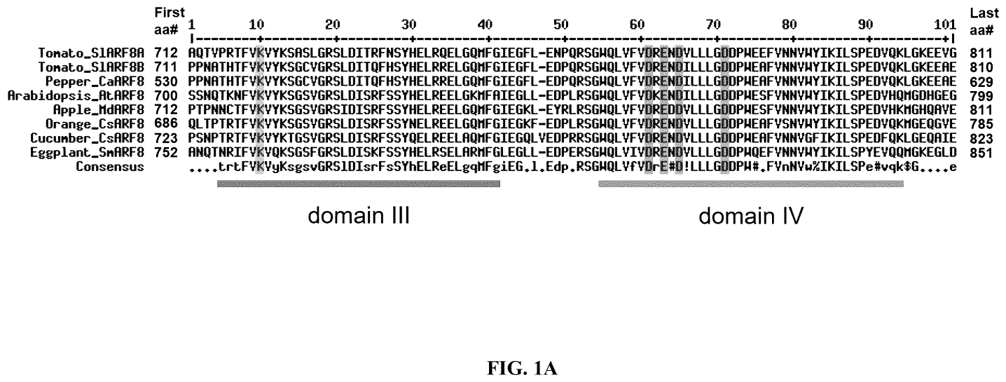

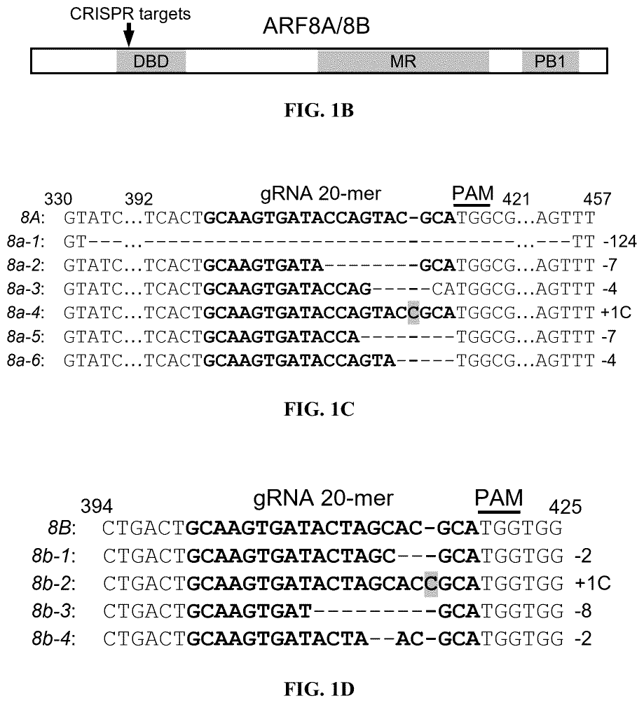

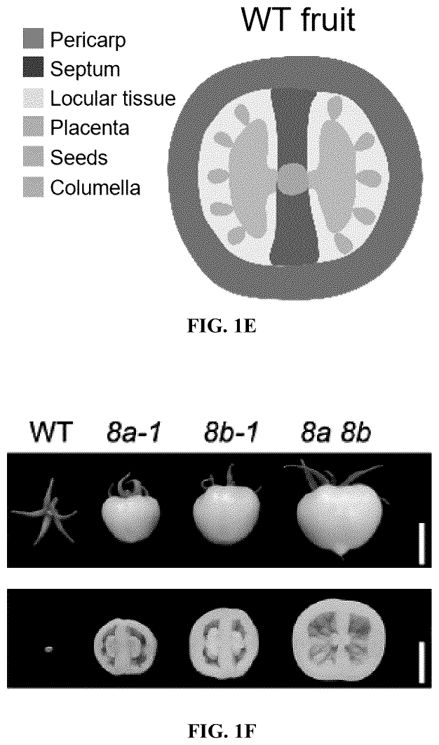

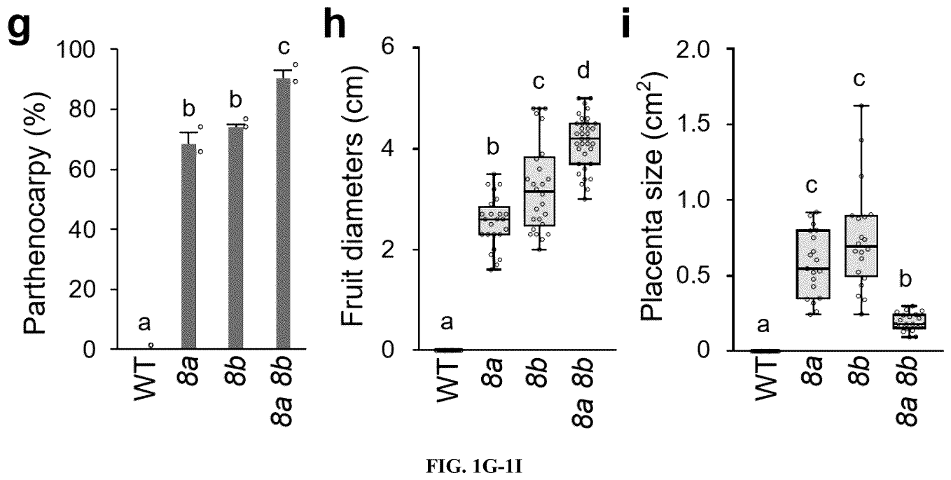

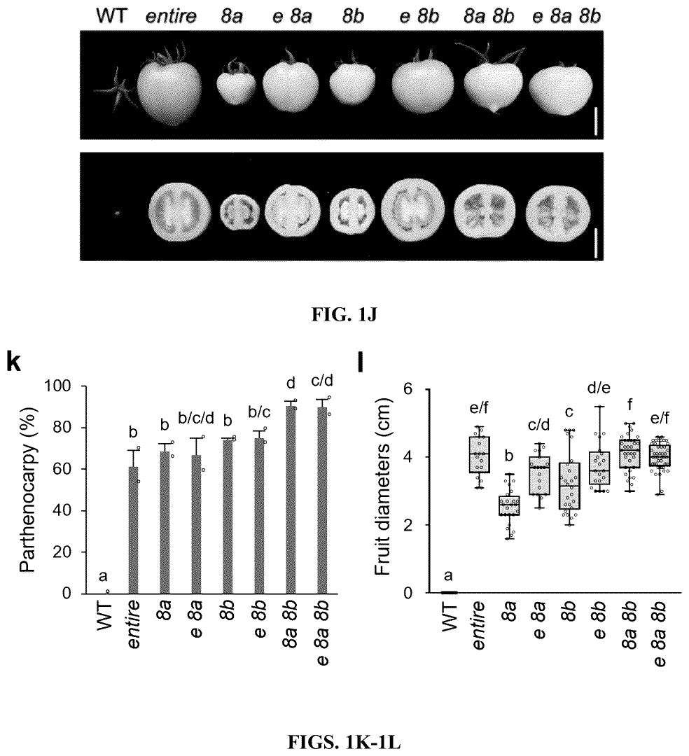

In some embodiments, the modified SlARF8A ortholog comprises Solanum lycopersicum SlARF8B comprising a deletion in amino acids 715-803 of SEQ ID NO: 83; Arabidopsis thaliana AtARF8 comprising a deletion in amino acids amino acids 704-792 of SEQ ID NO: 84; Capsicum annuum CaARF8 comprising a deletion in amino acids 534-622 of SEQ ID NO: 85; Solanum melongena SmARF8 comprising a deletion in amino acids 756-844 of SEQ ID NO: 86; Cucumis sativus CsARF8 comprising a deletion in amino acids 727-816 of SEQ ID NO: 87; Malus domestica MdARF8 comprising a deletion in amino acids 716-804 of SEQ ID NO: 88; or Citrus sinensis CsARF8 comprising a deletion in amino acids 690-778 of SEQ ID NO: 89. In some embodiments, the modified SlARF8A ortholog comprises Solanum lycopersicum SlARF8B and wherein amino acids 715-803 of SEQ ID NO: 83 or a portion thereof are deleted; wherein the modified SlARF8A ortholog comprises Arabidopsis thaliana AtARF8 and wherein amino acids 704-792 of SEQ ID NO: 84 or a portion thereof are deleted; wherein the modified SlARF8A ortholog comprises Capsicum annuum CaARF8 and wherein amino acids 534-622 of SEQ ID NO: 85 or a portion thereof are deleted; wherein the modified SlARF8A ortholog comprises Solanum melongena SmARF8 and wherein amino acids 756-844 of SEQ ID NO: 86 or a portion thereof are deleted; wherein the modified SlARF8A ortholog comprises Cucumis sativus CsARF8 and wherein amino acids 727-816 of SEQ ID NO: 87 or a portion thereof are deleted; wherein the modified SlARF8A ortholog comprises Malus domestica MdARF8 and wherein amino acids 716-804of SEQ ID NO: 88 or a portion thereof are deleted; or wherein the modified SlARF8A ortholog comprises Citrus sinensis CsARF8 and wherein amino acids 690-778 of SEQ ID NO: 89 or a portion thereof are deleted. In some embodiments, the modified SlARF8A ortholog is a truncated SlARF8B comprising amino acids 1-714 of SEQ ID NO: 83; a truncated AtARF8 comprising amino acids 1-703 of SEQ ID NO: 84; a truncated CaARF8 comprising amino acids 1-533 of SEQ ID NO: 85; a truncated SmARF8 comprising amino acids 1-755 of SEQ ID NO: 86; a truncated CsARF8 comprising amino acids 1-726 of SEQ ID NO: 87; a truncated MdARF8 comprising amino acids 1-715 of SEQ ID NO: 88; or a truncated CsARF8 comprising amino acids 1-689 of SEQ ID NO: 89. In some embodiments, the modified SlARF8A ortholog comprises one or more amino acid substitutions at positions corresponding K721, D771, E773, D775, or D781 of SEQ ID NO: 1, for example as shown in FIG. 1a.

In some embodiments, the ortholog is a functional ortholog that has retained a similar function in different species. In some embodiments, the ortholog is an ARF8A ortholog, for example, a SlARF8A ortholog, that comprises a PB1 domain, wherein the PB1 domain has at least 70%, 75%, 80%, 85%, 90%, 95%, or 99% sequence identity to the PB1 domain of SEQ ID NO: 1, and wherein the ortholog has least one activity of SEQ ID NO: 1, for example, protein-protein interaction between the PB1 domain of the ortholog and an Aux/IAA transcription repressor protein. In some embodiments, the ortholog is an ARF8 ortholog that comprises a PB1 domain, wherein the PB1 domain has at least 70%, 75%, 80%, 85%, 90%, 95%, or 99% sequence identity to the PB1 domain of any one of SEQ ID NOs: 90-97, as shown in FIG. 1a. In some embodiments, the ortholog is an ARF8 ortholog that comprises a PB1 domain, wherein the PB1 domain has at least 70%, 75%, 80%, 85%, 90%, 95%, or 99% sequence identity to the PB1 domain consensus sequence of SEQ ID NOs: 98, as shown in FIG. 1a. In some embodiments, the Aux/IAA transcription repressor protein is Solanum lycopersicum IAA9 or an ortholog thereof.

As used throughout, the term “nucleic acid” or “nucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar properties as the reference nucleic acid and are metabolized in a manner similar to naturally occurring nucleotides. A nucleic acid sequence can comprise combinations of deoxyribonucleic acids and ribonucleic acids. Such deoxyribonucleic acids and ribonucleic acids include both naturally occurring molecules and synthetic analogues. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

“Polypeptide,” “peptide,” and “protein” are used interchangeably herein to refer to a polymer of amino acid residues. As used herein, the terms encompass amino acid chains of any length, including full-length proteins, wherein the amino acid residues are linked by covalent peptide bonds.

The term “identity” or “substantial identity,” as used in the context of a polynucleotide or polypeptide sequence described herein, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 90%, 91%, 92%, 93%, 94%, 95%, 96%, 97%, 98%, or 99%, as compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, about 50 to about 200, and about 100 to about 150, in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85: 2444 (1988), by computerized implementations of these algorithms (e.g., BLAST), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25:3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10₋₂₀.

### Modifications

The genetically modified flowering plants provided herein comprise a modification, e.g., a mutation, in the PB1 domain of the ARF8A or ortholog thereof. As used herein, a modification or mutation can be an insertion, a deletion, or an amino acid substitution in the ARF8A or an ortholog thereof. In some embodiments, the modification includes one or more amino acid substitutions in the PB1 domain of the ARF8A or an ortholog thereof that disrupts the interaction between the ARF8A or an ortholog thereof and an auxin responsive protein, for example an IAA9 protein or ortholog thereof.

In some embodiments, the modification is one or more amino acid substitutions in the PB1 domain to replace a positively or negatively charged amino acid with an oppositely charged amino acid residue or a nonpolar amino acid to disrupt the charged interface that allows protein-protein interactions between the PB1 domains of ARF8A and one or more Aux/IAA transcription repressor proteins.

The PB1 domain in ARFs was formerly known as domains III and IV. This domain contains positively and negatively charged interfaces to allow protein-protein interaction. The key residues that contribute to these charged interfaces are the invariant lysine (K) residue in domain III (approximately, amino acids 716-752 of SEQ ID NO: 1) and the conserved acidic residues (D-x-D/E-x-D-xₙ-D/E in the OPCA motif) in domain IV (approximately amino acids 765-804 of SEQ ID NO: 1. Examples of substitutions that can be made in the PB1 domain include, but are not limited to one or more amino acid substitutions at positions K721, D771, E773, D775 and D781 of SlARF8A (SEQ ID NO: 1) or positions corresponding to K721, D771, E773, D775 and D781 of SEQ ID NO: 1 in the PB1 domain of an ortholog of SlARF8A.

In some embodiments, an amino acid substitution at one or more of positions D771,E773, D775 and D781 of SlARF8A (SEQ ID NO: 1) or positions corresponding to D771, E773, D775 and D781 of SEQ ID NO: 1 in the PB1 domain of an ortholog of SlARF8A replace a negatively charged amino acid with a nonpolar amino acid. In some embodiments, an amino acid substitution at one or more of position K721 of SlARF8A (SEQ ID NO: 1) or positions corresponding to K721 of SEQ ID NO: 1 in the PB1 domain of an ortholog of SlARF8A replace a positively charged amino acid with nonpolar amino acid. In some embodiments, an amino acid residue at one or more of the above-referenced positions can be substituted for an amino acid residue having an opposite charge.

In some embodiments, the modification is an amino acid substitution that introduces a stop codon into the ARF8A or an ortholog thereof which results in truncation or deletion of the C-terminus of the ARF8A or ortholog thereof, for example, truncation or deletion of the PB1 domain or a portion thereof. In some embodiments, the nucleic acid sequence encoding the ARF8A (one or both alleles of arf8a) or an ortholog thereof can be modified to introduce a frameshift mutation resulting in a truncation or deletion of the C-terminus of the ARF8A or ortholog thereof, for example, truncation or deletion of the PB1 domain or a portion thereof.

In other embodiments, the modification is a deletion or truncation of the entire PB1 domain or a portion thereof. In some embodiments, the deletion in the ARF8A or an ortholog thereof is a deletion of the amino acid corresponding to amino acids 716-804 of SEQ ID NO: 1 or a portion thereof. In some embodiments, the deletion is a deletion of amino acids 716-804 of SEQ ID NO: 1 (SlARF8A) or a portion thereof. In some embodiments, the deletion is a partial deletion of the PB1 domain where the amino acids corresponding to domain IV of the PB1 domain in SEQ ID NO: 1 (amino acids 765-804) are deleted. In some embodiments, amino acids 716-804 of SEQ ID NO: 1, amino acids 717-804 of SEQ ID NO: 1, amino acids 718-804 of SEQ ID NO: 1, amino acids 719-804 of SEQ ID NO: 1, amino acids 720-804 of SEQ ID NO: 1, amino acids 721-804 of SEQ ID NO: 1, amino acids 722-804 of SEQ ID NO: 1, amino acids 723-804 of SEQ ID NO: 1, amino acids 724-804 of SEQ ID NO: 1, or amino acids 725-804 of SEQ ID NO: 1 are deleted in SlARF8A. In some embodiments, the modified SlARF8A is a truncated SlARF8A comprising or consisting of amino acids 1-716 of SEQ ID NO: 1.

It is understood that when describing deletions of particular amino acid sequences in SEQ ID NO: 1, this also includes deletions of the corresponding amino acid sequences in an ortholog of SEQ ID NO: 1. In some embodiments, the deletion is a C-terminal deletion of at least 25, 30, 35, 40, 45, 50, 60, 70, 75, or 85 amino acids of the PB1 domain, for example, a C-terminal deletion of 25, 30, 35, 40, 45, 50, 60, 70, 75, or 85 amino acids of amino acids 716-804 of SEQ ID NO: 1 or the amino acid sequence corresponding to amino acids 716-804 of SEQ ID NO: 1.

In some embodiments, the genetically modified flowering plant does not comprise a modified ARF8B or ortholog thereof, for example, an inactive SlARF8B. In some embodiments, the genetically modified flowering plant expresses wildtype SlARF8B and/or comprises a wildtype SLARF8B gene.

Also contemplated are conservative amino acid substitutions in the modified ARF8A or ortholog thereof described above (i.e., variant sequences). By way of example, conservative amino acid substitutions can be made in one or more of the amino acid residues, for example, in one or more lysine residues of any of the polypeptides provided herein. One of skill in the art would know that a conservative substitution is the replacement of one amino acid residue with another that is biologically and/or chemically similar. The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine(S), Threonine (T); and
8) Cysteine (C), Methionine (M).

In some embodiments, the one or more lysine residues of a polypeptide described herein are substituted with one or more arginine residues.

By way of example, when an arginine to serine is mentioned, also contemplated is a conservative substitution for the serine (e.g., threonine). Nonconservative substitutions, for example, substituting a lysine with an asparagine, are also contemplated.

### Methods for Modification of Plants

In some embodiments, the genome of one or more flowering plant cells is modified by introducing a nucleic acid sequence encoding a modified plant protein (for example, an ARF8A protein comprising a deletion of the entire PB1 domain or a portion thereof) into one or more plant cells, as described in the Examples. ARF8A-NT, described in the Examples, is a construct comprising a truncated SlARF8A polypeptide consisting of amino acids 1-716 of SEQ ID NO: 1.In other words, amino acids 717-844 of SEQ ID NO: 1 are deleted in the truncated SlARF8A polypeptide produced by this construct.

In some embodiments, a recombinant nucleic acid encoding any of the modified ARF8A polypeptides described herein can be included in expression cassettes or constructs for expression in a plant. The cassette will include 5′ and 3′ regulatory sequences operably linked to a recombinant nucleic acid provided herein that allows for expression of the modified polypeptide. The cassette may additionally contain at least one additional gene or genetic element to be cotransformed into the organism. Where additional genes or elements are included, the components are operably linked. Alternatively, the additional gene(s) or element(s) can be provided on multiple expression cassettes. Such an expression cassette is provided with a plurality of restriction sites and/or recombination sites for insertion of the polynucleotides to be under the transcriptional regulation of the regulatory regions. The expression cassette may additionally contain a selectable marker gene. The expression cassette will include in the 5′-3′ direction of transcription, a transcriptional and translational initiation region (i.e., a promoter), a polynucleotide of the invention, and a transcriptional and translational termination region (i.e., termination region) functional in the organism of interest, i.e., a plant or bacteria. The promoters of the invention are capable of directing or driving expression of a coding sequence in a host cell, for example, a plant cell. The regulatory regions (i.e., promoters, transcriptional regulatory regions, and translational termination regions) may be endogenous or heterologous to the host cell or to each other. In some examples, an endogenous ARF8A promoter (for example, an SlARF8A promoter) can be used to drive expression of the modified ARF8A. As used herein, “heterologous” in reference to a sequence is a sequence that originates from a foreign species, or, if from the same species, is substantially modified from its native form in composition and/or genomic locus by deliberate human intervention.

Various methods can be used to introduce a sequence of interest into a host cell, for example, a plant cell. “Introducing” is intended to mean presenting a nucleic acid sequence to the host cell in such a manner that the sequence gains access to the interior of a cell. The methods disclosed herein do not depend on a particular method for introducing a sequence into a host cell only that the sequence gains access to the interior of at least one cell. Methods for introducing nucleic acid sequences into plants are known in the art including, but not limited to, stable transformation methods, transient transformation methods, and virus-mediated methods.

“Stable transformation” is intended to mean that the nucleotide construct introduced into a host cell or plant integrates into the genome of the host cell or plant and is capable of being inherited by the progeny thereof. “Transient transformation” is intended to mean that a polynucleotide is introduced into the host cell or plant and does not integrate into the genome of the host cell or plant or a polypeptide is introduced into a host cell or plant.

Transformation protocols as well as protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway et al. (1986) Biotechniques 4:320-334), electroporation (Riggs et al. (1986) Proc. Natl. Acad. Sci. USA 83:5602-5606, Agrobacterium-mediated transformation (U.S. Pat. Nos. 5,563,055 and 5,981,840), direct gene transfer (Paszkowski et al. (1984) EMBO J. 3:2717-2722), biolistic transformation (Klein et al. (1987) Nature 327:70-73), polyethylene glycol precipitation (Paszkowski et al. (1984) Embo J. 3: 2717-2722), and ballistic particle acceleration (see, for example, U.S. Pat. Nos. 4,945,050; 5,879,918; 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926); and Lecl transformation (WO 00/28058).

Methods are known in the art for the targeted insertion of a polynucleotide at a specific location in the plant genome. In one embodiment, the insertion of the polynucleotide at a desired genomic location is achieved using a site-specific recombination system. See, for example, International Application Nos. WO99/25821, WO99/25854, WO99/25840, WO99/25855, and WO99/25853, all of which are herein incorporated by reference in their entirety. Other methods include the use of targeted nucleases, for example, an RNA-guided nuclease, a transcription activator-like effector nuclease (TALEN), a zinc finger nuclease (ZFN), or a megaTAL (MT). See, for example, Gao et al. (2010) Plant Journal 1:176-187; and Zhang et al. (2018) Genome Biol. 19: 210. Base editing can also be used to modify the genome of one or more plant cells. See, for example, Rees and Liu (2018) Nat. Rev. Genet. 19 (12): 770-788.

The CRISPR/Cas9 system, an RNA-guided nuclease system that employs a Cas9endonuclease, can be used to edit the genome of a host cell or organism. The “CRISPR/Cas” system refers to a widespread class of bacterial systems for defense against foreign nucleic acid. CRISPR/Cas systems are found in a wide range of eubacterial and archaeal organisms. CRISPR/Cas systems include type I, II, and III sub-types. Wild-type type II CRISPR/Cas systems utilize an RNA-mediated nuclease, for example, Cas9, in complex with guide and activating RNA to recognize and cleave foreign nucleic acid. Guide RNAs having the activity of both a guide RNA and an activating RNA are also known in the art. In some cases, such dual activity guide RNAs are referred to as a single guide RNA (sgRNA).

As used herein, the term “Cas9” refers to an RNA-mediated nuclease (e.g., of bacterial or archeal orgin, or derived therefrom). Exemplary RNA-mediated nucleases include the foregoing Cas9 proteins and homologs thereof. Other RNA-mediated nucleases include Cpf1 (See, e.g., Zetsche et al., Cell, Volume 163, Issue 3, p759-771, 22 Oct. 2015) and homologs thereof.

Cas9 homologs are found in a wide variety of eubacteria, including, but not limited to bacteria of the following taxonomic groups: Actinobacteria, Aquificae, Bacteroidetes-Chlorobi, Chlamydiae-Verrucomicrobia, Chlroflexi, Cyanobacteria, Firmicutes, Proteobacteria, Spirochaetes, and Thermotogae. An exemplary Cas9 protein is the Streptococcus pyogenes Cas9 protein. Additional Cas9 proteins and homologs thereof are described in, e.g., Chylinksi, et al., RNA Biol. 2013 May 1; 10(5): 726-737; Nat. Rev. Microbiol. 2011 June; 9(6): 467-477; Hou, et al., Proc Natl Acad Sci U S A. 2013 Sep. 24;110(39):15644-9; Sampson et al., Nature. 2013 May 9;497(7448):254-7; and Jinek, et al., Science. 2012 Aug. 17;337(6096):816-21. Variants of any of the Cas9 nucleases provided herein can be optimized for efficient activity or enhanced stability in the host cell. Thus, engineered Cas9 nucleases are also contemplated. See, for example, “Slaymaker et al., “Rationally engineered Cas9 nucleases with improved specificity,” Science 351 (6268): 84-88 (2016)).

Transformed plant cells which are produced by any of the above transformation techniques can be cultured to regenerate a whole plant which possesses the transformed genotype and thus the desired phenotype, such as production of seedless fruit, compared to a control plant that was not transformed or transformed with an empty vector. Such regeneration techniques often rely on manipulation of certain phytohormones in a tissue culture growth medium, typically relying on a biocide and/or herbicide marker which has been introduced together with the desired nucleotide sequences. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally in Klee et al. Ann. Rev. of Plant Phys. 38:467-486 (1987).

### Methods for Making Plants

Provided herein are methods for producing any of the genetically modified plants described herein. In some embodiments, the method comprises a) modifying one or both alleles of an arf8a gene in one or more flowering plant cells to introduce a mutation into the nucleic acid sequence of the PB1 domain, as described above; and b) generating one or more flowering plants from the one or more flowering plant cells. In some embodiments, mutation is an insertion, a deletion or substitution of one or more nucleic acids in the genomic sequence encoding the PB1 domain

In some embodiments, the genome of the one or more flowering plant cells is modified by contacting the one or more flowering plant cells with an expression construct comprising a nucleic acid sequence encoding ARF8A or an ortholog thereof comprising a mutation in the PB1 domain, as described above. In some embodiments, the genome of the one or more flowering plant cells is modified by using Clustered Regularly Interspaced Short Palindromic Repeats (CRISPR) gene editing.

Some methods further comprising obtaining fruit from the one or more flowering plants. Some methods further comprising crossing the genetically modified flowering plant with a wildtype plant of the same species.

A recombinant plant produced by any of the methods provided herein is also provided. Also provided herein are the progeny of crosses (controlled or naturally occurring) derived from the genetically modified plants described herein, wherein the progeny is obtained without additional transformation or tissue culture propagation.

### Plant Characteristics

In some embodiments, the genetically modified flowering plants provided herein exhibit increased yield as compared to a control, for example, a wild type flowering plant of the same species. In some embodiments, the control is a reference value. As used herein, yield includes but is not limited to, the number of fruits, fruit size, the percentage of parthenocarpic fruit, placental growth, number of fruits per inflorescence, harvest index, or a combination thereof. In some embodiments, the genetically modified flowering plants provided herein exhibit similar yield as compared to wild type flowering plants of the same species, for example. In some embodiments, the increase in yield is at least a 5%, 10%, 15%, 20%, 25%, 30%, 35%, 40%, 45%, 50%, 55%, 60%, 65%, 70%, 75%, 80%, 90%, 95%, 100%, 150%, 200%, 250%, 300%, 350%, 400%, 500%, 550%, 600%, 650%, 700%, 750%, 800%, 850%, 900%, 950%, or a 1000% increase as compared to a control.

As described in the Examples, the fruit produced from the genetically modified flowering plants described herein are seedless, and surprisingly, exhibit placental growth similar to wildtype fruit. Therefore, in some embodiments, the fruit from the genetically modified flowering plants described herein, for example, a tomato, has at least 80%, 85%, 90%, 95%, or 100% placental growth as compared to a wildtype fruit from the same species.

In some embodiments, the genetically modified plants described herein have increased or enhanced stress tolerance as compared to a control plant, for example, a wildtype plant of the same species. Enhanced stress tolerance refers to an increase in the ability of a plant to decrease or prevent symptoms associated with one or more stresses. The stress can be a biotic stress that occurs as a result of damage done to plants by other living organisms such as a pathogen (for example, bacteria, viruses, fungi, parasites), insects, nematodes, weeds, cultivated or native plants. The stress can also be an abiotic stress such as extreme temperatures (high or low), high winds, drought, salinity, chemical toxicity, oxidative stress, flood, tornadoes, wildfires, radiation and exposure to heavy metals. Therefore, increased stress tolerance can be, but is not limited to, an increase in disease resistance, an increase in drought resistance, an increase in heat tolerance, an increase in low temperature tolerance (cold stress), and/or an increase in heavy metal tolerance, to name a few.

Plants with increased or enhanced stress tolerance can be selected or identified in several ways. One of ordinary skill in the art will recognize that the following methods are but a few of the possibilities. One of skill in the art will also recognize that stress responses of plants vary depending on many factors, including the type of stress and plant used. Generally, enhanced stress tolerance is measured by the reduction or elimination of symptoms associated with a particular stress when compared to a control plant. This reduction or decrease does not have to be complete, as this reduction can be about a 10, 20, 30, 40, 50, 60, 70, 80, 90 or 100% reduction when compared to a control plant.

For example, one of skill in the art can select plants with increased abiotic (e.g., drought, high or low temperatures, heavy metal, UV, salt) resistance by determining the rates of photosynthesis and stomatal conductance of a plant under stress conditions (See, for example, Hozain et al. Tree Physiology 30: 32-44 (2010); Frost et al. PLoS One 7(8):e44467 (2012)). Tolerance to stresses can also be gauged by production of reactive oxygen species (ROS), by increased expression of marker genes (such as genes encoding heat-shock protein in the case of heat tolerance), or by electrolyte leakage assays of the membrane (Wahid et al. Environmental and Experimental Botany 61(3):199-223(2007); Bajji et al. Plant Growth Regulation 36:61-70 (2002)).

In another example, a method of selecting plants with increased disease resistance is to determine resistance of a plant to a specific plant pathogen (see, e.g., Agrios, Plant Pathology (Academic Press, San Diego, CA) (1988)). Enhanced resistance is measured by the reduction or elimination of disease symptoms when compared to a control plant. In some cases, however, enhanced resistance can also be measured by the production of the hypersensitive response (HR) of the plant (see, e.g., Staskawicz et al. Science 268(5211): 661-7 (1995)). Plants with enhanced disease resistance can produce an enhanced hypersensitive response relative to control plants.

In some embodiments, the amount and/or activity of ARF8A mRNA or ARF8A protein produced by the plant is increased as compared to a control. In some embodiments, for example, in a tomato plant, the amount and/or activity of SlARF8A mRNA or SlARF8A protein produced by the plant is increased as compared to a control. In some embodiments the increase in activity is a decrease in the interaction (i.e., binding) between ARF8A protein and an auxin response corepressor protein, for example, an Aux/IAA transcription repressor protein. In some embodiments the increase in activity is because there is a decrease in the interaction (i.e., binding) between SlARF8A and SIAA9. This decreased interaction, as discussed above, does not have to be complete, as this decrease can be about a 10, 20, 30, 40, 50, 60, 70, 80, 90 or 100% reduction when compared to the amount of interaction between the proteins in a control plant.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### EXAMPLES

### Example I. Class A-Auxin Response Factor Analysis

### A. Plant Materials, Growth Conditions and Statistical Analysis

Tomato (Solanum lycopersicum) cultivar Moneymaker (MM) was used as WT in this study. The entire (iaa9) mutant was backcrossed to MM five times from their original Alisa Craig background (Zhang et al. A single-base deletion mutation in SlIAA9 gene causes tomato (Solanum lycopersicum) entire mutant. J. Plant Res. 120, 671-678, (2007)). arf5-1 and CR-arf7 mutants were backcrossed to MM five times from their original M82 background (Hu et al. The Interaction between DELLA and ARF/IAA Mediates Crosstalk between Gibberellin and Auxin Signaling to Control Fruit Initiation in Tomato. Plant Cell 30, 1710-1728, (2018)). CR-arf8a and CR-arf8b mutants were generated in this study. Genotyping primers for entire, arf5-1 and CR-arf7 were described previously (Hu et al.). Genotyping primers for the CR-arf8a, CR-arf8b mutants are listed in Table 2. In this study, all single and higher order arf mutants are homozygous, except when specified to be heterozygous.

TABLE 2
List of Primers

| | | | | |
|---|---|---|---|---|
| | | SEQ ID | | |
| Primer | Sequence | NO: | Used for | Notes |

Cloning

| | | | | |
|---|---|---|---|---|
| ARF8A-6 | 5′- | 3 | Making | 20-mer |
| | GTCGAAGTAGTGATTGCAAGTGATACCAGTACGCAGTTT | | pCR4- | sequence in |
| | TAGAGCTAGAA | | ARF8A | bold |
| ARF8A-7 | TTCTAGCTCTAAAACTGCGTACTGGTATCACTTGCAATCA | 4 | gRNA | |
| | CTACTTCGAC | | | |
| ARF8B-6 | 5′- | 5 | Making | 20-mer |
| | GTCGAAGTAGTGATTGCAAGTGATACTAGCACGCAGTTT | | pCR4- | sequence in |
| | TAGAGCTAGAA | | ARF8B | bold |
| ARF8B-7 | TTCTAGCTCTAAAACTGCGTGCTAGTATCACTTGCAATCA | 6 | gRNA | |
| | CTACTTCGAC | | | |
| pCUT3 | 5′- | 7 | Cloning of | |
| gRNA1-5′ | GACTCTAGACACGGGGGGTTTAAGCTTTCGTTGAACAA | | ARF8A | |
| | CGGAAAC | | gRNA to | |
| pCUT3 | 5′-CTTGTTTAAACGTTTAAACAAAAAAAGCACCGACTCGG | 8 | pCUT3 | |
| gRNA1-3′ | | | | |
| pCUT3 | 5′-TTTGTTTAAACGTTTAAACAAGCTTTCGTTGAACAACGG | 9 | Cloning of | |
| gRNA2-5′ | | | ARF8B | |
| pCUT3 | AGCAACTCATTACAACTTGTTTCAAAAAAAGCACCGACTC | 10 | gRNA to | |
| gRNA2-3″ | GGTG | | pCUT3 | |
| ARF8A-13 | 5′-GGTCCTTGTTATTGTCGACCGTTTTCG | 11 | Cloning of | Native |
| | | | ARF8A 3 kb | Sall/Xmal |
| ARF8A-14 | 5′- | 12 | promoter to | |
| | TCAGACCCGGGATTTTTAAACACCCTTAAATTATGATTAT | | pBS | |
| | CC | | | |
| ARF8A-15 | 5′-GTACGTCGACATGAAGCTTTCAACATCAGGAATGGG | 13 | Cloning of | Sall/Sacl |
| | | | ARF8A | |
| ARF8A-16 | 5′-GTACGAGCTCTCAGTAATCAAGTGATCCTATAGATG | 14 | cDNA to | |
| | | | pBS | |
| HA-7 | 5′- | 15 | Cloning of | BamHI/Eco |
| | GATCCGTACCCATACGATGTTCCAGATTACGCTTACCCAT | | 2xHA tag to | |
| | ACGATGTTCCAGATTACGCTG | | pDONR207- | |
| HA-8 | 5′- | 16 | 3FLAG-RGA | RI |
| | AATTCAGCGTAATCTGGAACATCGTATGGGTAAGCGTAAT | | (RZ) | |
| | CTGGAACATCGTATGGGTACG | | | |
| 3FLAG2H | 5′- | 17 | Cloning of | Apal/Sall; |
| A-1 | ATCAGGGGCCCGGGATGGACTACAAAGACCATGACGGT | | 3FLAG2HA | Xmal is |
| | GATTA | | tag to pBS- | after Apal |
| 3FLAG2H | 5′- | 18 | ARF8A | for next step |
| A-2 | ATTCGTCGACAGCGTAATCTGGAACATCGTATGGGTAAG | | cDNA | |
| | CG | | | |
| ARF8B-11 | 5′-AGCTGTCGACATGAAGCTTTCAACATCAGGAATGGG | 19 | Cloning of | Sall/Sacl |
| | | | ARF8B | |
| | | | cDNA to | |
| ARF8B-12 | 5′-AGCTGAGCTCAGTACTCCAGCGATCCAAGAGATG | 20 | pBS-3F2H- | |
| | | | ARF8A | |
| | | | cDNA | |
| ARF8B-13 | 5′-GGACTAAAAGCCTCACTACGGTCCGTG | 21 | Cloning of | Xbal/Xmal |
| | | | ARF8B | |
| | | | 3.5 kb | |
| ARF8B-14 | 5′-AGCT CCCGGG | 22 | promoter to | |
| | AATTATATTTTATTTATTTATTTAAAAACCC | | pBS | |
| pSLT1- | 5′-AAACTTAATTAATTTAAATCCCGGGATCC | 23 | Cloning of | Pmel/Xhol |
| MCS-F | | | MCS (Pmel- | |
| | | | Pacl-Swal- | |
| | | | Xmal- | |
| pSLT1- | 5′-TCGAGGATCCCGGG ATTTAAATTAATTAAGTTT | 24 | BamHI-Xhol) | |
| MCS-R | | | to pCUT3 to | |
| | | | make pSLT1 | |
| 3FLAG-6 | 5′- | 25 | Cloning of | Gibson |
| | GTTTAAACTTAATTAATTTATGGACTACAAAGACCATGAC | | 3F2H- | |
| | GGTGATT | | ARF8B to | |
| ARF8B-21 | 5′- | 26 | pSLT1 | assembly |
| | TCGAGGATCCCGGGATTTTCAGTACTCCAGCGATCCAAG | | | |
| | AGATG | | | |
| ARF8B-22 | 5′- | 27 | Cloning of | Gibson |
| | CTCTAGACACGGGGTGGTTTCTAGACTTTTAAATTTTGAA | | ARF8B | |
| | GTGTTAT | | 3.5 kb | |
| ARF8B-23 | 5′- | 28 | promoter to | assembly |
| | GTCCATAAATTAATTAAGTTTAATTATATTTTATTTATTTAT | | pSLT1- | |
| | TTAAAAACC | | 3F2H- | |
| | | | ARF8B | |
| | | | cDNA | |
| ARF5-5 | 5′-TCGAGGATCCCGGGATTT | 29 | Cloning of | Gibson |
| | TTAGTTTGGGAATTCTGATGTTGATCC | | 3F-ARF5 to | assembly |
| | | | pSLT1, with | |
| | | | 3FLAG-6 | |
| ARF5-6 | 5′- | 30 | Cloning of | Gibson |
| | CTCTAGACACGGGGTGGTTTCTCTATCATGACTTCTCGC | | ARF5 3.7 kb | |
| | GTCATTTC | | promoter to | |
| ARF5-7 | 5′- | 31 | pSLT1-3F- | assembly |
| | GTCCATAAATTAATTAAGTTTAAAAACCTCTGTGATTCCCT | | ARF5 cDNA | |
| | TTACATCA | | | |
| ARF7-1 | 5′-GTACGTCGACATGAAAGCGCCATCAAACGGATATC | 32 | Cloning of | Sall/Xmal |
| | | | ARF7 cDNA | |
| ARF7-8 | 5′-GTATCCCCGGGTCAATTAAATGATGCAGCTGAGTTGTC | 33 | to pBS | |
| 3FLAG-1 | 5′- | 34 | Cloning of | Apal/Sall; |
| | CGGGATGGACTACAAAGACCATGACGGTGATTATAAAGA | | 3xFLAG tag | Xmal is |
| | TCATGACATCGATTACAAGGATGACGATGACAAGG | | to pBS- | |
| 3FLAG-2 | 5′- | 35 | ARF7 cDNA | after Apal |
| | TCGACCTTGTCATCGTCATCCTTGTAATCGATGTCATGAT | | | for next step |
| | CTTTATAATCACCGTCATGGTCTTTGTAGTCCATCCCGGG | | | |
| | CC | | | |
| ARF7-6 | 5′-GCACGTCGACGCCCTATCTTAGGGTGTCTGTAAAG | 36 | Cloning of | Sall/Sacl; |
| | | | ARF7 4 kb | Xmal is |
| ARF7-7 | 5′- | 37 | promoter to | before Sacl |
| | ATGCGAGCTCCCCGGGTTCAGCAAAAAATCCACTTAAGT | | pGPTV-Kan | for next step |
| | TGAATTAC | | | |
| IAA9-3 | 5′- ATAAGTCGACCGTAGGTGGGTGGGTGGGGGTG | 38 | Cloning of | Sall/Sacl; |
| | | | IAA9 3 kb | Xmal is |
| IAA9-4 | 5′- | 39 | promoter to | before Sacl |
| | CGGAGAGCTCCCGGGCACAATTACAGATCACTCTAATAA | | pBS | for next step |
| | TCCTGC | | | |
| ARF8A-19 | 5′-GTACGAGCTCTTAGGGCACAGTTTGTGCATCAGCTTGC | 40 | Cloning of | Sall/Sacl |
| | | | ARF8A-NT | |
| | | | to pBS, with | |
| | | | ARF8A-15 | |
| ARF8B-15 | 5′- | 41 | Cloning of | Sall/Sacl |
| | GTACGAGCTCTTAGGTTGCATTTGGTGGATCAATTTGTCC | | ARF8B-NT | |
| | | | to pBS, with | |
| | | | ARF8B-11 | |
| 3FLAG-3 | 5′- | 42 | Cloning | |
| | TCAGGTCGACATGGACTACAAAGACCATGACGGTGATTA | | ARF8A/ARF | |
| | | | 8A- | |
| | | | NT/TPL1N1 | |
| | | | 00-IAA9 to | |
| | | | pCR8 | |
| TPL1-1 | 5′-CTAGGTCGACATGTCATCTCTCAGTAGAGAGCTTG | 43 | Cloning of | Dral/Sall |
| | | | TPL1 N- | |
| | | | terminal | |
| TPL1-2 | 5′-AAACGTCGACATCCTTTACAAGAATTTCAACACCC | 44 | 100aa to | |
| | | | pENTR1A- | |
| | | | IAA9 | |

Mutant Analysis

| | | | |
|---|---|---|---|
| ARF8A-10 | 5-TGCTGTTGCATTTTCAGCAAGAAC | 45 | genotyping arf8a crispr |
| | | | mutants. WT/8a-1 PCR |
| | | | products: 197 bp/73 bp.For |
| ARF8A-11 | 5-AAGACAGTGGACCAGTTATATCACC | 46 | other alleles, after Rsal |
| | | | digestion, WT 116 + 81 bps, |
| | | | 8a-2 to 8a-6 alleles uncut. |
| ARF8B-8 | 5′-CTCTGTGTTGGATTTCAGGAAG | 47 | genotyping arf8b crispr |
| | | | mutants. After Mlul |
| ARF8B-10 | 5′-CGACGAGGGACAGAAAAGCCACCACG | 48 | digestion, WT 118 + 28 bps, |
| | | | 8b-1 to 8b-4 alleles uncut. |

RT-qPCR analysis

| | | |
|---|---|---|
| MADS2 Frt/ | 5′-GAGAAGCAATTGGAGCAGAGTGTC/5′- | 49/113 |
| Rrt | CAGCTGTAAGGGCTCTCTCCCTT | |
| HOX.1 Frt/ | 5′-GAGGCTCTCCTAGCTCATAACAAC/5′- | 50/114 |
| Rrt | GATTGATTCTGTTGGCCCCTTTCC | |
| HSF Frt/ | 5′-GAAGAAGAGGAGGAGGCCAATTG/5′- | 51/115 |
| Rrt | CCCATGTGACATGTTCATAGCCAC | |
| XTH7 Frt/ | 5′-GGGAAGCTGACGACTGGGCTAC/5′- | 52/116 |
| Rrt | GCTGGTCCTGGCATTGCACATCC | |
| TPX1 Frt/ | 5′-GCCCTGGAGTTGTTTCTTGTGC/5′- | 53/117 |
| Rrt | CCAGTTGGCACATTCCAGAAAGG | |
| NR Frt/ | 5′-ACCCTCAACACTCAACCCGAG/5′- | 54/118 |
| Rrt | TCCCTTGTGAGGTTTGCACAC | |
| ABAH Frt/ | 5′-GTGGTGTACATGCTTGTCCAGGG/5′- | 55/119 |
| Rrt | TACCAGATCCTACCACTTCCCAC | |
| ERF4 Frt/ | 5′-CAGTCCAGTTTTCGACAGCTCCGC/5′- | 56/120 |
| Rrt | CCCCCATGGCCTTCTCCTTACC | |
| IAA9 Frt/ | 5′-GAGAGGCCTTCTGCTGTGAA/5′- | 57/121 |
| Rrt | CAACCAACAACCTGTGCCTT | |

ChIP-qPCR analysis

| | | |
|---|---|---|
| AG1 - | 5′-GTCTCATGATTGGCATAGAAGAG/5′- | 58/122 |
| 1600F/R | GCCTTCCCTTAGGATATGATTC | |
| AG1 - | 5′-TGGATCTGTCTATGATAGTGAGAG/5′- | 59/123 |
| 800F/R | GTAAACTCCACCTCTCCATCCC | |
| AG1 - | 5′-CCCATCCCTCACCCAAAATTCTC/5′- | 60/124 |
| 500F/R | GGATGATCACTCATTTCTTGGAC | |
| MADS2 - | 5′-CAGCATCAGCATCTAACAACG/5′- | 61/125 |
| 1700F/R | CTACCTTATTTGTTGGCTTGAAT | |
| MADS2 - | 5′-GATGAATGCACCTCTCGAGAAA/5′- | 62/126 |
| 1200F/R | GGGTGCCTTGTGGTGACAGAT | |
| MADS2 - | 5′-GATGTAGATGCAGATCCAAGTCC/5′- | 63/127 |
| 300F/R | CTGGATGAAGTACAACCTCTTGAG | |
| AGL6 - | 5′-GGCAATAGGTGGAGGGATC/5′- | 64/128 |
| 2400F/R | GGTAGTTTCATATGACATGC | |
| AGL6 - | 5′-GTCTGAGAAGACATCGCAAC/5′- | 65/129 |
| 1400F/R | CCACGGTTCAAATCCAAGACG | |
| AGL6 - | 5′-GGTTGCTAAATCAGCCCATAT/5′- | 66/130 |
| 600F/R | GTAGCATCCAACTAATGTCT | |
| XTH7 - | 5′-CCTGTCTAAAAGCCTAGAGACAAC/5′- | 67/131 |
| 2400F/R | GGACAAGTCACGAATAACAATCCC | |
| XTH7 - | 5′-CCTATGGCTCCTCCATTGGGC/5′- | 68/132 |
| 1500F/R | GCAAGACACTCCCGTGACAAGC | |
| XTH7 - | 5′-CATAGGGTACACCTAGCTAGATGT/5′- | 69/133 |
| 600F/R | GATCTCTCTTTGAATGGTGTCTCC | |
| GA20ox1 - | 5′-CGTCCCAATGTCTCATTCAGTGG/5′- | 71/134 |
| 2800F/R | AAGCGCAATATGATATACAATTCCG | |
| GA20ox1 - | 5′-GCGCCATCCAAAGCGGACCCA/5′- | 72/70 |
| 1500F/R | GCTTATACCCGTAGCACGGTAC | |

Tomato plants were grown in greenhouse with 16 h day/8 h night light cycle as described previously (Hu et al.). Parthenocarpy test was done as described previously in Hu et al., with some modifications: −2 DAA flowers on mature tomato plants (2-6 weeks after first flowering) were emasculated and recorded fruit growth 5 weeks later; fruit diameter of at least 1 cm was considered as parthenocarpy. For fruit yield analysis, the number of flowers were counted for each young flower cluster of ˜two-month-old plants, and fruits developed on these clusters were harvested individually and weighed when they were at the red ripe stage. Whole plant phenotypes including plant height, internode numbers and length, and leaf morphology were recorded 5 weeks after sowing. For 2,4-D treatment, 10 μl of 100 μM 2,4-D, or mock solvent (5% MeOH, 0.1% Tween-20) was applied to −2 DAA ovaries of emasculated flowers. All statistical analyses were performed using Student's t-test.

### B. Plasmid Construction

Primers for plasmid construction are listed in Table 2. PCR-amplified DNA fragments in constructs were sequenced to ensure that no mutations were present. Detailed information on plasmid construction is described in Table 3A and Table 3B.

TABLE 3A
Constructs for Tomato

| | | Antibiotic | Insert/PCR | | | | Cloning |
|---|---|---|---|---|---|---|---|
| Construct | Plasmid name | Resistance | product | Primers | Template | Plasmid backbone | method |
| ARF8A | pCR4-ARF8A | Kanamycin | ARF8A 20-mer | ARF8A-6/-7 | TOPO GFP | TOPO GFP | site- |
| gRNA | gRNA | | gRNA | | gRNA | gRNA | directed |
| | | | | | | | mutagenesis |
| ARF8B | pCR4-ARF8B | Kanamycin | ARF8B 20-mer | ARF8B-6/-7 | TOPO GFP | TOPO GFP | site- |
| gRNA | gRNA | | gRNA | | gRNA | gRNA | directed |
| | | | | | | | mutagenesis |
| ARF8A/8B | pCUT3-ARF8A/ | Spectinomycin | ARF8A/8B | pCUT3 | pCR4-ARF8A/8B | pCUT3 | Gibson |
| gRNA | 8B gRNA | | gRNAs | gRNA1/2- | gRNA | | assembly |
| | | | | 5′ + -3′ | | | |
| ARF8A | pBS-ARF8A | Ampicillin | ARF8A 3 kb | ARF8A- | WT genomic | pBlueScriptII | Sall/Xmal |
| promoter | promoter | | promoter | 13/-14 | DNA | | |
| ARF8A | pGPTV-ARF8A | Kanamycin | ARF8A 3 kb | — | pBS-ARF8A | pGPTV-Kan | Sall/Xmal |
| promoter | promoter | | promoter | | promoter | | |
| ARF8A | pBS-ARF8A | Ampicillin | ARF8A cDNA | ARF8A- | pCR8-ARF8A | pBlueScriptII | Sall/Sacl |
| cDNA | cDNA | | | 15/-16 | | | |
| 3xFLAG- | pDONR207- | Gentamycin | 2xHA tag | HA-7/-8 | annealed oligos | pDONR207- | BamHI/Ec |
| 2xHA | 3xFLAG-2xHA | | | | | 3xFLAG-RGA | ORI |
| 3F2H-ARF8A | pBS-3F2H- | Ampicillin | 3xFLAG-2xHA | 3FLAG2H | pDONR207- | pBS-ARF8A cDNA | Apal/Sall |
| cDNA | ARF8A cDNA | | tag | A-1/-2 | 3xFLAG-2xHA | | |
| Pro_{ARF8A}:3F2 | pGPTV-Pro_{ARF8A}:3F2H- | Kanamycin | 3F2H-ARF8A | | pBS-3F2H- | pGPTV-ARF8A | Xmal/Sacl |
| H-ARF8A | ARF8A | | cDNA | | ARF8A cDNA | promoter | |
| 3F2H-ARF8B | pBS-3F2H-ARF8B | Ampicillin | ARF8B cDNA | ARF8B- | pCR8-ARF8B | pBS-3F2H-ARF8A | Sall/Sacl |
| cDNA | cDNA | | | 11/-12 | | cDNA | |
| ARF8B | pBS-ARF8B | Ampicillin | ARF8B 3.5 kb | ARF8B- | WT genomic | pBlueScriptII | Xbal/Xmal |
| promoter | promoter | | promoter | 13/-14 | DNA | | |
| pSLT1 MCS | pSLT1 | Spectinomycin | pSLT1 MCS | pSLT1- | annealed oligos | pCUT3 | Pmel/Xhol |
| | | | | MCS-F/-R | | | |
| 3F2H-ARF8B | pSLT1-3F2H-ARF8B | Spectinomycin | 3F2H-ARF8B | 3FLAG- | pBS-3F2H- | pSLT1 | Gibson |
| cDNA | cDNA | | cDNA | 6/ARF8B-21 | ARF8B cDNA | | assembly |
| Pro_{ARF8B}:3F2 | pSLT1-Pro_{ARF8B}:3F2H- | Spectinomycin | ARF8B 3.5 kb | ARF8B- | pBS-ARF8B | pSLT1-3F2H- | Gibson |
| H-ARF8B | ARF8B | | promoter | 22/-23 | promoter | ARF8B cDNA | assembly |
| ARF5 cDNA | pEG-3F-ARF5 | Kanamycin | ARF5 cDNA | — | pCR8-ARF5 | pEG-3xFLAG | Gateway LR |
| 3F-ARF5 | pSLT1-3F-ARF5 | Spectinomycin | 3xFLAG-ARF5 | 3FLAG- | PEG-3F-ARF5 | pSLT1 | Gibson |
| | | | | 6/ARF5-5 | | | assembly |
| Pro_{ARF5}:3F- | pSLT1-Pro_{ARF5}:3F-ARF5 | Spectinomycin | ARF5 3.7 kb | ARF5-6/-7 | WT genomic | pSLT1-3F-ARF5 | Gibson |
| ARF5 | | | promoter | | DNA | | assembly |
| ARF7 cDNA | pBS-ARF7 cDNA | Ampicillin | ARF7 cDNA | ARF7-1/-8 | pENTR1A-ARF7 | pBlueScriptII | Sall/Xmal |
| 3xFLAG-ARF7 | pBS-3F-ARF7 | Ampicillin | 3xFLAG tag | 3FLAG-1/-2 | annealed | pBS-ARF7 | Apal/Sall |
| cDNA | cDNA | | | | oligos | cDNA | |
| ARF7 | pBS-ARF7 | Ampicillin | ARF7 4 kb | ARF7-6/-7 | WT genomic | pBlueScriptII | Sall/Sacl |
| promoter | promoter | | promoter | | DNA | | |
| ARF7 | pGPTV-ARF7 | Kanamycin | ARF7 4 kb | — | pBS-ARF7 | pGPTV-Kan | Sall/Sacl |
| promoter | promoter | | promoter | | promoter | | |
| Pro_{ARF7}:3F- | pGPTV-Pro_{ARF7}:3F-ARF7 | Kanamycin | 3xFLAG-ARF7 | — | pBS-3F-ARF7 | pGPTV-ARF7 | Xmal |
| ARF7 | | | cDNA | | cDNA | promoter | |
| IAA9 cDNA | pBS-IAA9 | Ampicillin | IAA9 cDNA | — | pENTR1A-IAA9 | pBlueScriptII | Sall/EcoRV |
| 3F2H-IAA9 | pBS-3F-IAA9 | Ampicillin | 3xFLAG-2xHA | 3FLAG2H | pDONR207- | pBS-IAA9 | Apal/Sall |
| cDNA | | | tag | A-1/-2 | 3xFLAG-2xHA | | |
| IAA9 | pBS-IAA9prom | Ampicillin | IAA9 3 kb | IAA9-3/-4 | WT genomic | pBlueScriptII | Sall/Sacl |
| promoter | | | promoter | | DNA | | |
| IAA9 | pGPTV-IAA9prom | Kanamycin | IAA9 3 kb | — | pBS-IAA9prom | pGPTV-Kan | Sall/Sacl |
| promoter | | | promoter | | | | |
| ProlAA9:3F2 | pGPTV-IAA9:3F-IAA9 | Kanamycin | 3F2H-IAA9 | — | pBS-3F-IAA9 | pGPTV-IAA9prom | Xmal |
| H-IAA9 | | | cDNA | | | | |
| 3F2H- | pBS-3F2H-ARF8A-NT | Ampicillin | ARF8A-NT | ARF8A- | pCR8-ARF8A | pBS-3F2H-ARF8A | Sall/Sacl |
| ARF8A-NT | | | (aa 1-716) | 15/-19 | | cDNA | |
| Pro_{ARF8A}:3F2 | pGPTV-Pro_{ARF8A}:3F2H- | Kanamycin | ARF8A-NT | — | pBS-3F2H- | pGPTV-ARF8A | Xmal/Sacl |
| H-ARF8A-NT | ARF8A-NT | | (aa 1-716) | | ARF8A-NT | promoter | |
| 3F2H- | pBS-3F2H-ARF8B-NT | Ampicillin | ARF8B-NT | ARF8B- | pCR8-ARF8B | pBS-3F2H-ARF8B | Sall/Sacl |
| ARF8B-NT | | | (aa 1-715) | 11/-15 | | cDNA | |
| 3F2H- | pGPTV-3F2H-ARF8B-NT | Kanamycin | ARF8B-NT | — | pBS-3F2H- | pGPTV-Kan | Xmal/Sacl |
| ARF8B-NT | | | (aa 1-715) | | ARF8B-NT | | |
| Pro_{ARF8B}:3F2 | pGPTV-Pro_{ARF8B}:3F2H- | Kanamycin | ARF8B 3.5 kb | — | pBS-ARF8B | pGPTV-3F2H- | Xbal/Xmal |
| H-ARF8B-NT | ARF8B-NT | | promoter | | promoter | ARF8B-NT | |

TABLE 3B
Constructs for Yeast Auxin Response Circuit

| | | Antibiotic | Insert/PCR | | | Plasmid | Cloning |
|---|---|---|---|---|---|---|---|
| Construct | Plasmid name | Resistance | product | Primers | Template | backbone | method |
| 3F2H-ARF8A | pCR8-3F2H-ARF8A | Spectinomycin | 3F2H-ARF8A | 3FLAG- | pBS-3F2H-ARF8A | pCR8/GW/ | TOPO TA |
| | cDNA | | cDNA | 3/ARF8A-2 | cDNA | TOPO | cloning |
| Pro_{ADH1}:3F2H- | pGP8A-3F2H- | Ampicillin | 3F2H-ARF8A | — | pCR8-3F2H- | pGP8A- | Gateway LR |
| ARF8A | ARF8A cDNA | | cDNA | | ARF8A cDNA | ccdB | |
| 3F2H-ARF8A-NT | pCR8-3F2H- | Spectinomycin | 3F2H-ARF8A- | 3FLAG- | pBS-3F2H- | pCR8/GW/ | TOPO TA |
| | ARF8A-NT | | NT | 3/ARF8A-19 | ARF8A-NT | TOPO | cloning |
| Pro_{GPD}:3F2H- | pGP5G-3F2H- | Ampicillin | 3F2H-ARF8A- | — | pCR8-3F2H- | pGP5G- | Gateway LR |
| ARF8A-NT | ARF8A-NT | | NT | | ARF8A-NT | ccdB | |
| TPL1^{N100}-IAA9 | pENTR1A- | Kanamycin | TPL1 N-terminal | TPL1-1/-2 | WT cDNA | pENTR1A- | Dral/Sall |
| | TPL1^{N100}-IAA9 | | 100aa | | | IAA9 | |
| 3F-TPL1^{N100}-IAA9 | PEG-3F-TPL1^{N100}- | Kanamycin | TPL1^{N100}-IAA9 | — | pENTR1A- | PEG- | Gateway LR |
| | IAA9 | | | | TPL1^{N100}-IAA9 | 3xFLAG | |
| 3F-TPL1^{N100}-IAA9 | PCR8-3F-TPL1^{N100}- | Spectinomycin | 3F-TPL1^{N100}- | 3FLAG- | PEG-3F-TPL1^{N100}- | pCR8/GW/ | TOPO TA |
| | IAA9 | | IAA9 | 3/IAA9-2 | IAA9 | TOPO | cloning |
| Pro_{GPD}:3F- | pGP4G-3F- | Ampicillin | 3F-TPL1^{N100}- | — | pCR8-3F- | pGP4G- | Gateway LR |
| TPL1^{N100}-IAA9 | TPL1^{N100}-IAA9 | | IAA9 | | TPL1^{N100}-IAA9 | ccdB | |

### C. Plant Transformation

The P_{GENE}:tag-GENE (including SlARF8A, SlARF8B, SlARF5, SlARF7 , SlIAA9), P_{GENE}:tag-GENE-NT (including SlARF8A-NT and SlARF8B-NT) and CRISPR Cas9-ARF8A/ARF8B constructs were introduced into tomato (MM) through agrobacterium-mediated plant transformation with strain GV3101 pMP90 (Bajwa et al. Identification and functional analysis of tomato BRI1 and BAK1 receptor kinase phosphorylation sites. Plant Physiol 163, 30-42, (2013)). For P_{GENE}:tag-GENE transformation, T0 lines containing a single insertion were identified by their T1 seedlings showing a 3:1 ratio of kanamycin-resistant versus kanamycin-sensitive segregation patterns. For each construct, 3-5 independent homozygous lines with WT-like phenotype were tested by immunoblot analysis to identify representative spatial expression patterns in ovary, and a representative line (i.e. 3F-ARF5 #C7-3-3; 3F-ARF7 #C4-7-6; 3F2H-ARF8A #C5-24-4; 3F2H-ARF8B #C8-4-1; 3F2H-IAA9 #C6-4A-6) was chosen for final expression analysis and complementation test. Most of the 31 3F2H-ARF8A T0 lines had normal fruit and seed production, while one line #C5-5 was completely parthenocarpic. This line had the highest ARF8A protein level among all T0s and was named ARF8A-OE. For P_{GENE}:tag-GENE-NT transformation, 25 ARF8A-NT and 9 ARF8B-NT T0 lines were generated. These T0 lines were used directly for phenotyping, protein analysis and RT-qPCR analysis. The representative lines used in this study are 3F2H-ARF8A-NT #G1-2 and 3F2H-ARF8B-NT #G2-3. For CRISPR Cas9-ARF8A/8B transformation, arf8A and arf8b mutant alleles were amplified by PCR using genotyping primers in T0 lines and sequenced to identify the molecular lesions. To remove possible off-target mutation(s) and the Cas9 transgene, a representative T0 mutant was backcrossed to WT once.

### D. Synthetic Auxin Response System in Yeast

The published minimal ARC^{SC} (for ARC in yeast) contains a single AtTIR1/AFB, AtTPL^{N100}-AtIAA (truncated TPL-IAA fusion), AtARF and an auxin-responsive promoter:Venus (output reporter) (Pierre-Jerome et al. Recapitulation of the forward nuclear auxin response pathway in yeast. Proc Natl Acad Sci U S A 111, 9407-9412, (2014)). The Venus signal can be quantified by flow cytometry. New ARC^{SC} with tomato components including 3F-SlTPL1^{N100}-SlIAA9 fusion, 3F-SlARF8A or 3F-SlARF8A-NT were generated. All tomato genes are expressed using constitutive yeast promoters. The system described herein also contains a synthetic auxin-response promoter P3_2x fused to the fluorescent protein Venus coding sequence (P3_2x:Venus) (Pierre-Jerome et al.). pGP8A-3F-SlARF8A, pPG5G-3F-SlARF8A-NT constructs were separately transformed into the yeast reporter line in yeast strain MATa. Then pGP4G-3F-SlTPL1^{N100}-SlIAA9 was transformed to ARF8A or 8A-NT-containing lines or P3_2x:Venus line alone. All constructs were incorporated in yeast chromosome permanently. Venus fluorescence was recorded by flow cytometry with an FACSCanto Flow Cytometer (BD Bioscience). The median value of 10,000 cells was recorded as one reading.

### E. Immunoblot Analysis

Whole ovary or dissected ovary tissues were extracted by grinding in 2x Laemmli buffer (Bio-Rad) and boiling for 10 min. After centrifugation, lysates were separated by SDS-PAGE, and detected by immunoblotting with either an HRP-conjugated anti-FLAG antibody (Sigma A8592 clone M2, for detection of 3F2H-ARF8A, 3F2H-ARF8B, 3F-ARF5, 3F-ARF7 with dilutions of 1:3,000, 1:2,000, 1:1,000 and 1:2,000, respectively) or a mouse anti-HA antibody (BioLegend #901503, for detection of 3F2H-IAA9, 3F2H-ARF8A-NT, 3F2H-ARF8B-NT with dilution of 1:1,000). As gel loading control, tubulin was detected with a mouse anti-tubulin antibody (Sigma T5168) at dilution of 1:500,000. HRP-conjugated donkey anti-mouse secondary antibody (Jackson ImmunoResearch) was used for anti-HA and anti-tubulin immunoblotting at dilution of 1:5,000 and 1:50,000, respectively.

### F. RNA-Seq Analysis

Total RNAs were purified from −2 DAA ovaries of WT and 8a-1 8b-1, as well as −2 DAA WT ovaries treated with 100 μM 2,4-D for 2/6/24 h or mock treated (5% methanol, 0.1% Tween 20). RNA-Seq cDNA libraries (three biological repeats) were prepared with the QuantSeq 3′mRNA-Seq library prep kit FWD for Illumina (Lexogen). DNA sequencing was performed with Illumina Next-Seq500 High-Output 75 bp SR. Sequence alignment and DE (differential expression) analysis were done online at lexogen.bluebee.com. Heatmap was created in RStudio with plotly package. Co-regulated genes among 8a 8b and +2,4-D 2/6/24 h gene lists were then identified (fold change >1.5; p<0.1 for 8a 8b vs. WT; p<0.05 for +2,4-D vs. mock). Venn Diagrams were made using online tool at InteractiVenn.net^{53.} GO analysis was performed using agriGO v2 toolkit (Tian et al. agriGO v2.0: a GO analysis toolkit for the agricultural community, 2017 update. Nucleic Acids Res 45, W122-W129, (2017)). Heatmap analysis was made in R language with the plotly package.

### G. RT-qPCR Analysis

Total RNAs from whole ovary or dissected ovary tissues were isolated with Quick-RNA MiniPrep kit (Zymo Research). First-strand cDNA was then synthesized using a Transcriptor First Strand cDNA Synthesis kit (Roche). qPCR analyses were performed using FastStart Essential DNA Green Master mix (Roche) and LightCycler 96 instrument (Roche). The PCR program was performed as described previously (Hu et al.). Three biological replicates from independent pools of tissues (2 technical repeats each) were included for each experiment. Primers for qPCR are either previously described (Hu et al.) or listed in Table 2.

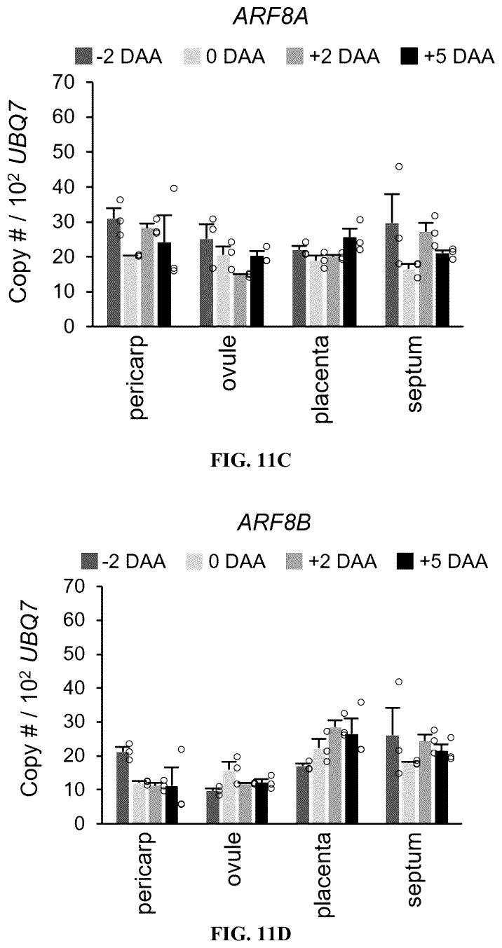

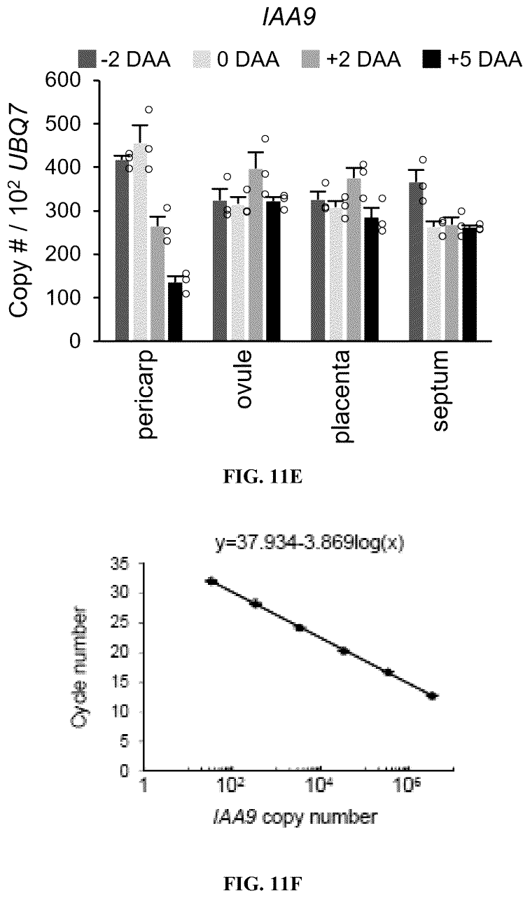

For absolute qPCR analysis, the qPCR standard curves of UBQ7 and SlARF genes were made previously (Hu et al.). For SlIAA9, linearized plasmid pENTRIA-IAA9 cDNA (Hu et al.) served as template for determining cDNA copy vs qPCR cycle number (FIG. 11F). With these standard curves, the cDNA copy numbers of UBQ7, SlARFs and SlIAA9 were determined according to their respective cycle numbers.

### H. ChIP-qPCR Analysis

Ovaries of the 3F2H-ARF8A transgenic line C5-24-4 (−2 and +5 DAA) and ARF8A-NT line (−2 DAA) were used for ChIP-qPCR. Experiments were performed as described (Hu et al). Primer sequences for qPCR testing are listed in Table 2.

### I. Accession Numbers

Sequence information for genes included throughout can be found in the Sol Genomics Network (https://solgenomics.net/) under the following g accession numbers: ARF8A (Solyc03g031970), ARF8B (Solyc02g037530), ARF5 (Solyc04g084210), ARF7 (Solyc07g042260), IAA9 (i.e. ENTIRE, Solyc04g076850), AG1 (Solyc02g071730), MADS2 (Solyc01g092950), AGL6 (Solyc01g093960), HOX.1 (Solyc01g010600), HSF (Solyc06g053950), ACO4 (Solyc02g081190), XTH7 (Solyc02g091920), TPX1 (Solyc07g052510), NR (Solyc11g013810), ABAH (Solyc04g078900), ERF4 (Solyc05g052030), GA20ox1 (Solyc03g006880), XTH15 (Solyc03g031800), HD52 (Solyc04g074700), ZF-HD1 (Solyc04g074990), MYB21 (Solyc02g067760), MYB77 (Solyc04g079360), CKX2 (Solyc10g017990), MIPS (Solyc04g054740), UBQ7 (Solyc10g005560), TPL1 (Solyc03g117360).

### J. Results

1. arf8a arf8b Produced Seedless Fruits With Reduced Placenta

SlARF8A (Solyc03g031970) and SlARF8B (Solyc02g037530) in tomato are the closest orthologs to Arabidopsis AtARF8, and their encoded protein sequences share 81% identity. To examine the role of SlARF8A and SlARF8B in fruit development, slarf8a, slarf8b knockout mutants were generated in the Moneymaker (MM) cultivar using the CRISPR/Cas9 technology. In a single tomato transformation experiment, each ARF8 gene was targeted by a unique guide RNA (gRNA) at its coding sequence within the DNA-binding domain (FIG. 1a). Several frame-shift null slarf8a, slarf8b alleles were identified in T0 lines by DNA sequence analysis (FIG. 1b-1c), and each T0 line contains two different mutant alleles for SlARF8A and SlARF8B. To obtain the slarf8A and slarf8b single and double mutants without the Cas-9/gRNAs transgene, a T0 line that contained arf8a-1/8a-2 arf8b-1/8b-2 alleles was backcrossed to WT (wild type). Two sets of single and double arf8a arf8b homozygous mutants were identified that contain two independent 8a and 8b alleles in the F2 generation: (1) arf8a-1, arf8b-1 and arf8a-1 arf8b-1; (2) arf8a-2, arf8b-2 and arf8a-2 arf8b-2. As shown below, these two sets of mutants showed consistent phenotypes. Therefore, arf8a-1 (8a-1), arf8b-1 (8b-1) and 8a-1 8b-1 (8a 8b) lines were used for further studies.

To examine fruit set without pollination, flowers of these arf8 mutants were emasculated to remove stamens and petals at 2 days before anthesis (−2 DAA), and their parthenocarpic fruit growth was recorded after 5 weeks when the fruits had reached their maximum size at the mature green stage. WT did not display any parthenocarpy, while 8a-1 and 8b-1 single mutants produced seedless fruits from ˜70% of unfertilized ovaries (FIG. 1e, 1f). The 8a 8b double mutant displayed even stronger parthenocarpy (˜90%) than single mutants, and its parthenocarpic fruit size was also bigger than the single mutants (FIG. 1e, 1g), indicating that SlARF8A and SlARF8B have overlapping functions in repressing fruit initiation and development. However, 8a 8b almost completely eliminated placenta growth (FIG. 1e, 1h), which is the tissue for ovules/seeds attachment (FIG. 1d). The 8a and 8b single mutants also showed reduced placenta growth with intermediate phenotypes between WT and the double mutant. The strong parthenocarpy and the lack of placenta growth in 8a 8b mutant suggest dual function of SlARF8A/8B in repressing fruit initiation and promoting placenta growth, respectively.

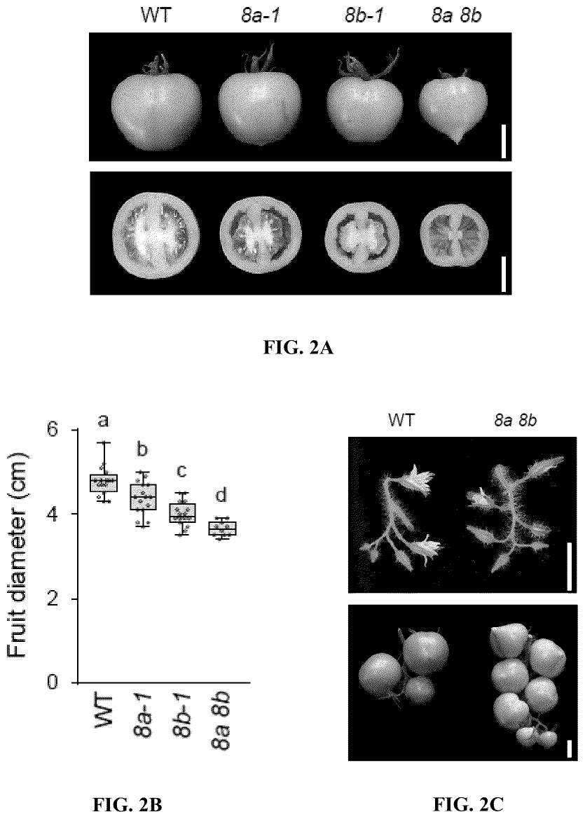

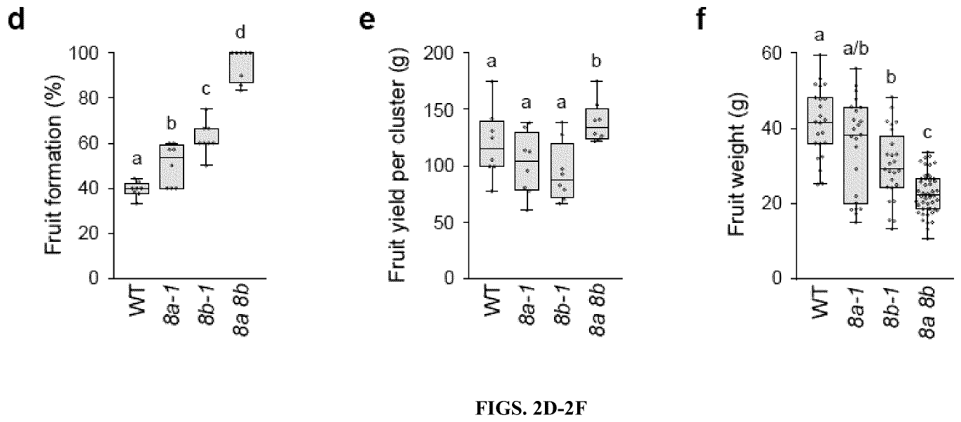

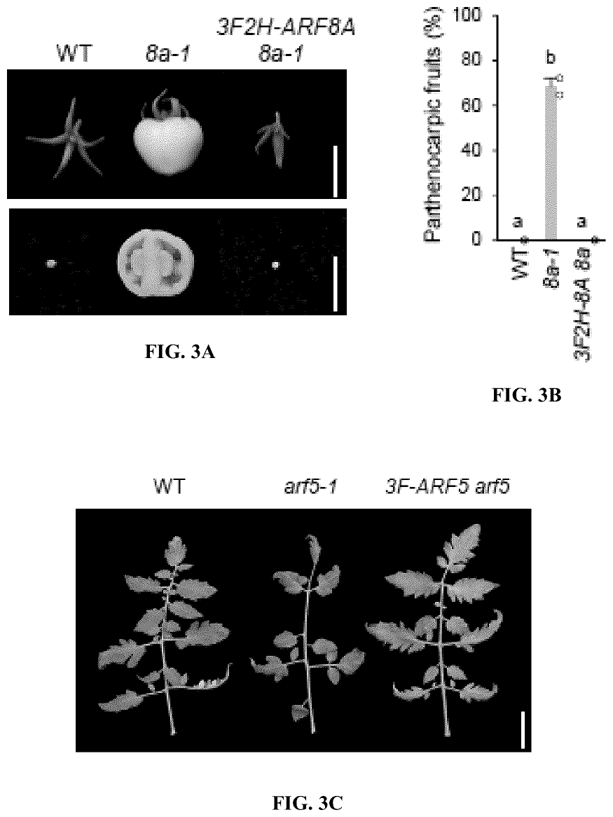

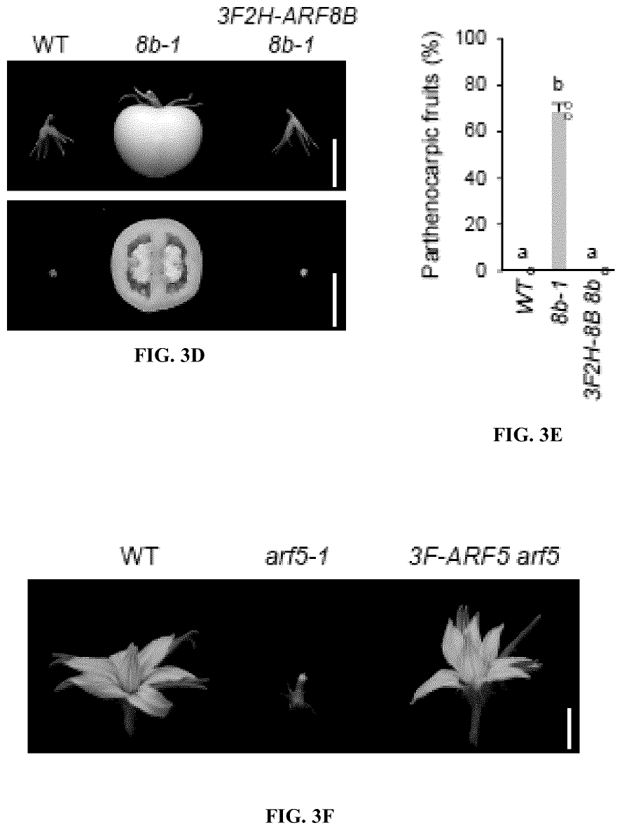

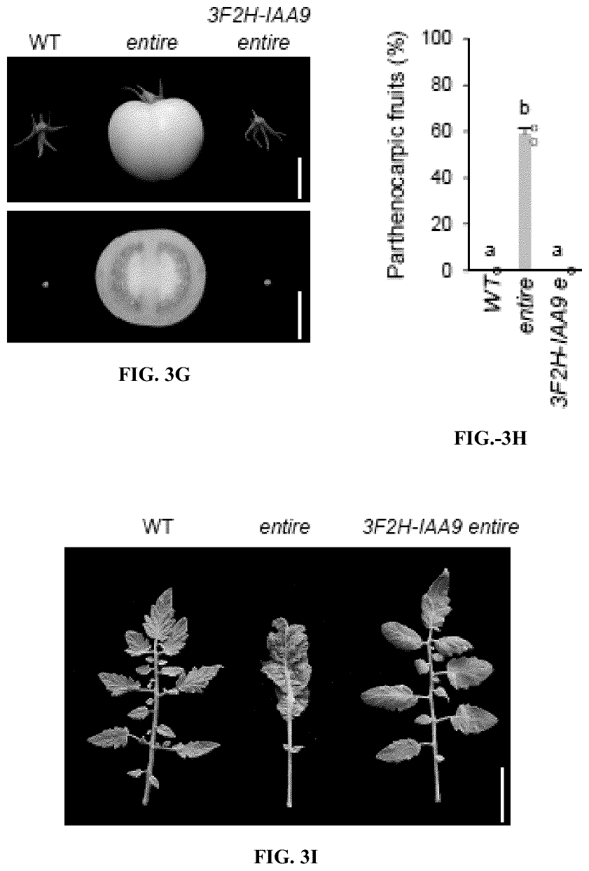

Phenotypes of mutant fruits after natural pollination were also examined, and it was found that the overall fruit sizes of 8a-1 and 8b-1 were smaller than WT (FIG. 2a-2b). The placenta and locular tissue in these single mutants were also reduced in comparison to WT. The fruit phenotypes (after fertilization) in 8a-1 and 8b-1 point to the possible activator function of ARF8in fruit growth. In contrast, the 8a 8b double mutant displayed obligatory parthenocarpy because it produced similar parthenocarpic fruits with or without emasculation (FIG. 1e and FIG. 2a), indicating the double mutant is either male- and/or female-sterile. The 8a 8b fruit size was further reduced from the 8a-1 or 8b-1 single mutant (FIG. 2b), indicating that both SlARF8A and SlARF8B promote fruit growth. It was also found that almost every flower of 8a 8b was able to form fruit (95%, FIG. 2c-2d) because of its high parthenocarpy frequency. In contrast, only 40% of WT flowers developed into fruits under the same growth condition. The total fruit yield (total weight) per cluster was also higher in 8a 8b, although average weight per fruit of 8a 8b was lower comparing to WT and arf8 single mutants (FIG. 2e-2f). To confirm that the phenotypes in 8a-1 and 8b-1 were caused respectively by the slarf8A or slarf8b crispr allele but not off-target mutations, P_{ARF8A}:3xFLAG-2xHA-ARF8A (3F2H-ARF8A) and P^{ARF8B}:3xFLAG-2xHA-ARF8B (3F2H-ARF8B) transgenic lines were made and crossed to 8a-1 or 8b-1 respectively. 3F2H-ARF8A and 3F2H-ARF8B transgenes rescued the strong parthenocarpy phenotype in 8a-1 and 8b-1 mutants, respectively (FIG. 3a-3e), verifying that 8a-1 and 8b-1 alleles lead to parthenocarpy.

As mentioned earlier, the placenta growth defect in 8a 8b is likely the consequence of missing the activator roles of SlARF8A/8B. But it could also result from the repressor functions of other class A-ARFs forming complexes with IAA9. To test the latter possibility, epistasis analysis was performed between 8a 8b and the sliaa9 null allele [entire (e)] to examine if removing SlIAA9function would restore placenta growth in 8a 8b. As shown in FIG. 1i, the emasculated entire mutant flowers produced large seedless fruits with well-developed placenta and locular gel-like tissue, whereas the e 8a 8b triple mutant displayed similar placenta defect as in 8a 8b, suggesting that ARF8A/8B are the major ARFs promoting placenta growth in tomato. However, the e 8a and e 8b double mutants displayed more placenta growth than the 8a-1 and 8b-1 single mutants, indicating that removing SlIAA9 repression can enhance the activator role of the remaining functional SlARF8A or 8B in placenta. These results thus confirmed that the slarf8A slarf8b double mutations are epistatic to sliaa9 in regulating placenta growth.

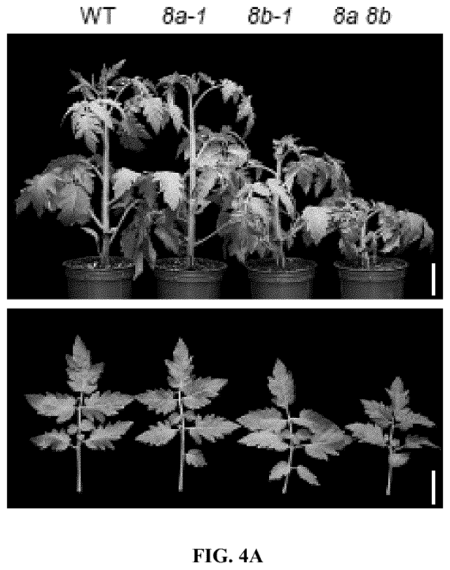

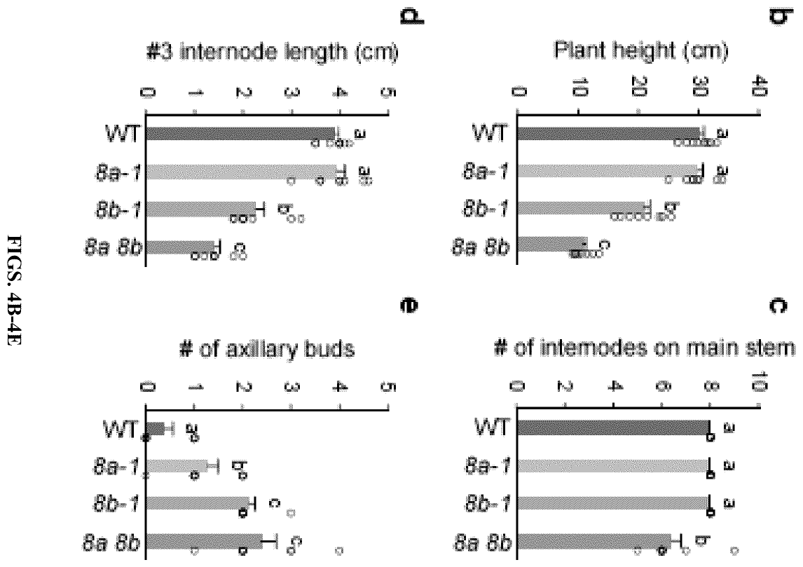

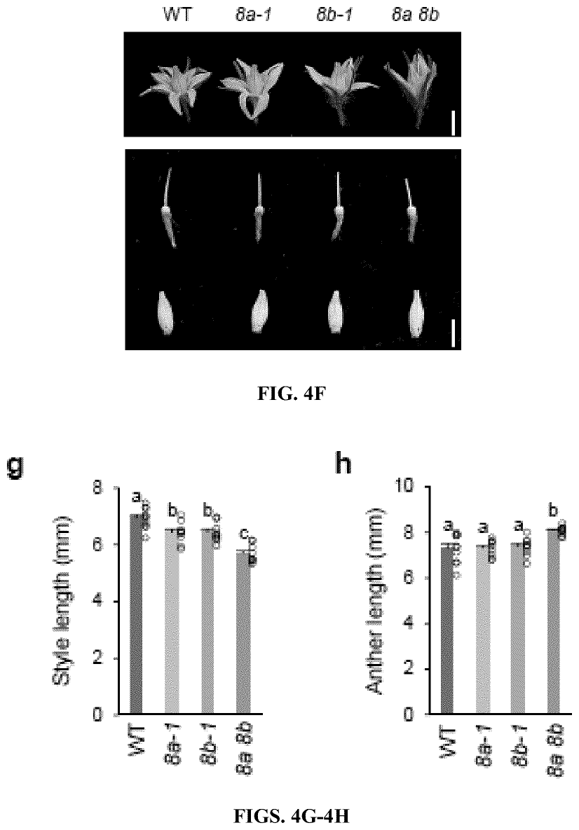

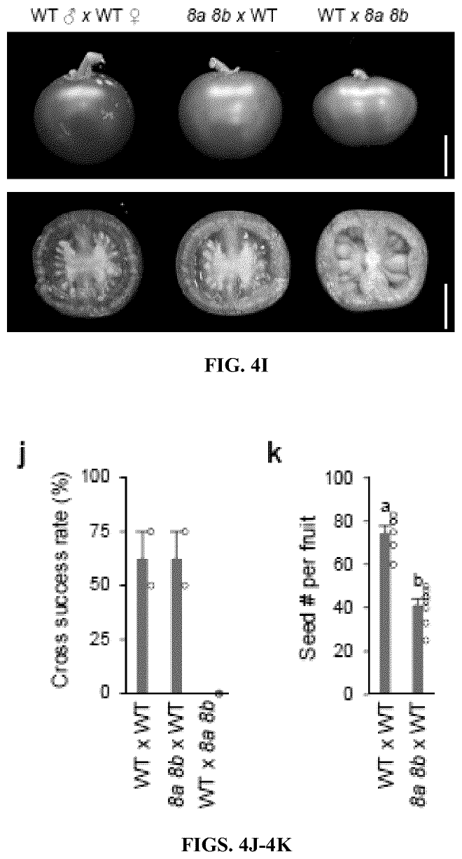

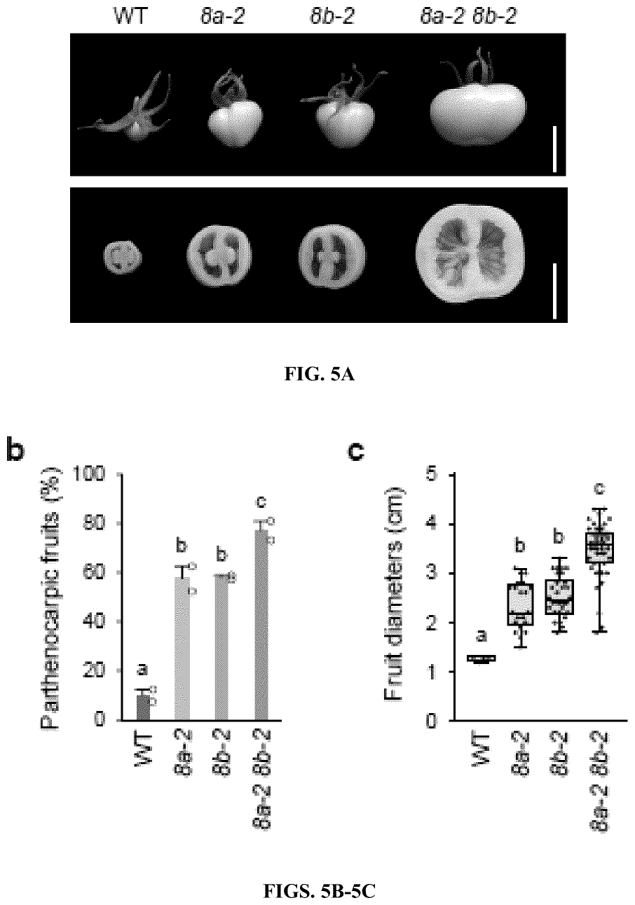

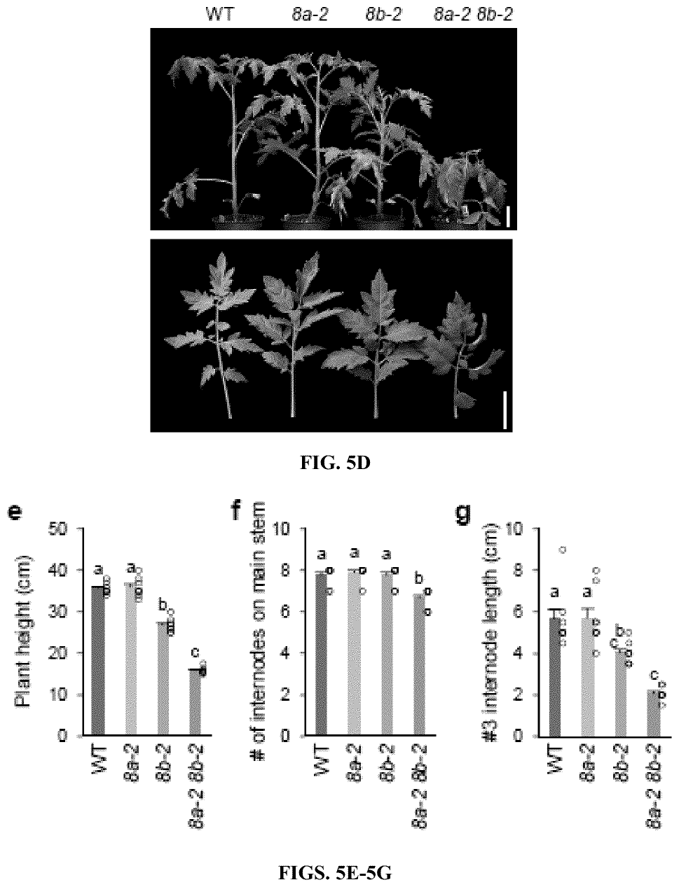

In addition to fruit development, the ARF8 mutants also showed additional defects in vegetative growth and in flower development. 8b-1 was shorter than WT and the 8a 8b double mutant was even shorter and produced smaller leaves than 8b-1 (FIG. 4a-4e), indicating ARF8A and ARF8B additively promote stem growth and leaf expansion. The 80-1 and 8b-1 mutations also additively reduced style length to 82% of WT, but increased anther cone length to 110% of WT (FIG. 4f-4h). These subtle defects may reduce pollination efficiency, but the total infertility in 8a 8b is likely caused by additional factors as shown by the results of reciprocal crosses between WT and 8a 8b (FIG. 4i-4k). It was found that 8a 8b pollen was as efficient as the WT pollen to trigger fruit set when WT ovary was the recipient. However, the fruits from 8a 8b (♂)×WT only produced 50% of seeds than those from WT x WT, suggesting reduced pollen viability in 8a 8b. In contrast, WT (♂)×8a 8b cross produced seedless fruits just like 8a 8b itself, indicating 8a 8b is female-sterile. The smaller leaf, shorter stem/style and female-sterile phenotypes in 8a 8b are consistent with the phenotype previously reported for the MIR167 transgenic tomato line with reduced ARF6/8 expression (targeted by overexpression of AtMIR167a). These results suggest that SlARF8A/8B mainly act as activators for promoting auxin-mediated flower development, except that they may inhibit anther cone growth. The parthenocarpy and whole plant phenotypes in arf8a-1, arf8b-1 mutants were reproducible in arf8a-2, arf8b-2 single and double mutants (FIG. 5), confirming that the mutant phenotypes in 8a 8b are due to slarf8a and slarf8b mutations.

### 2. Class A-ARFs Exhibited Dual Function in Fruit Development

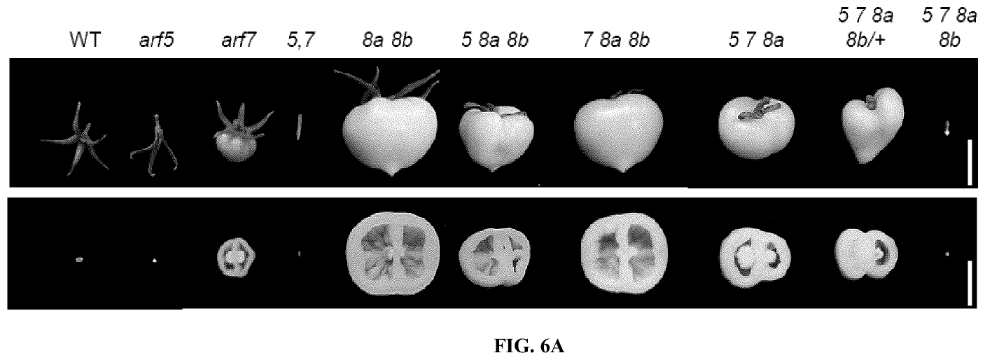

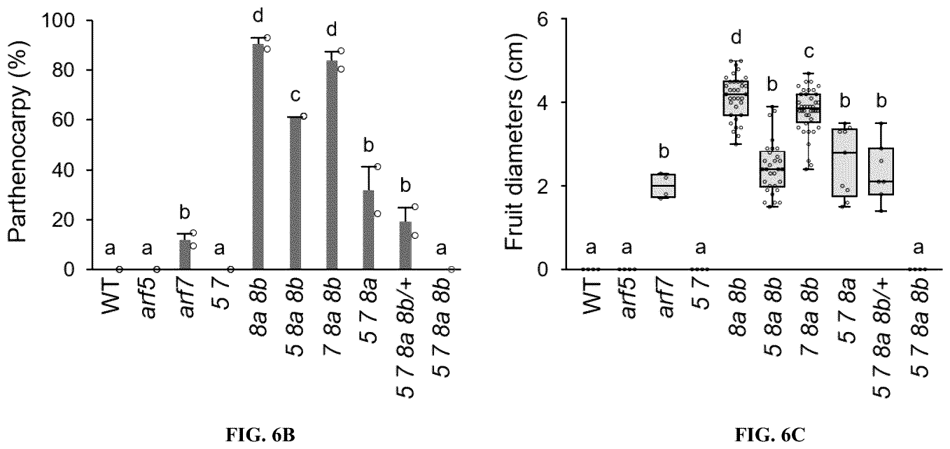

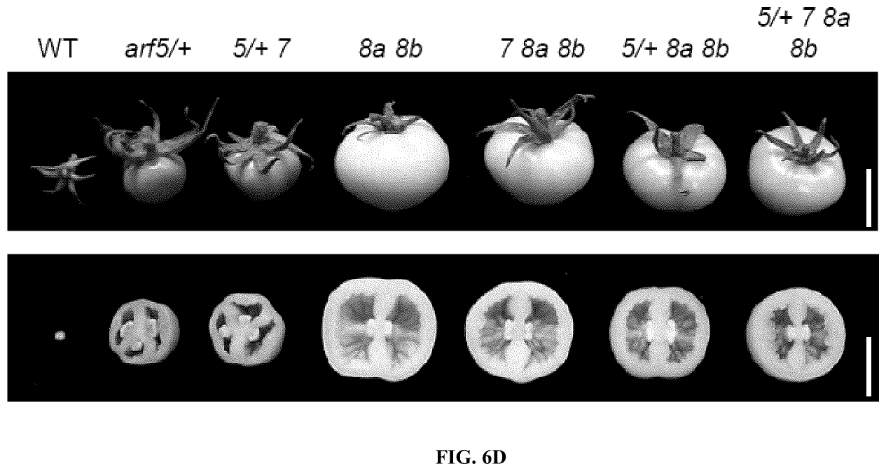

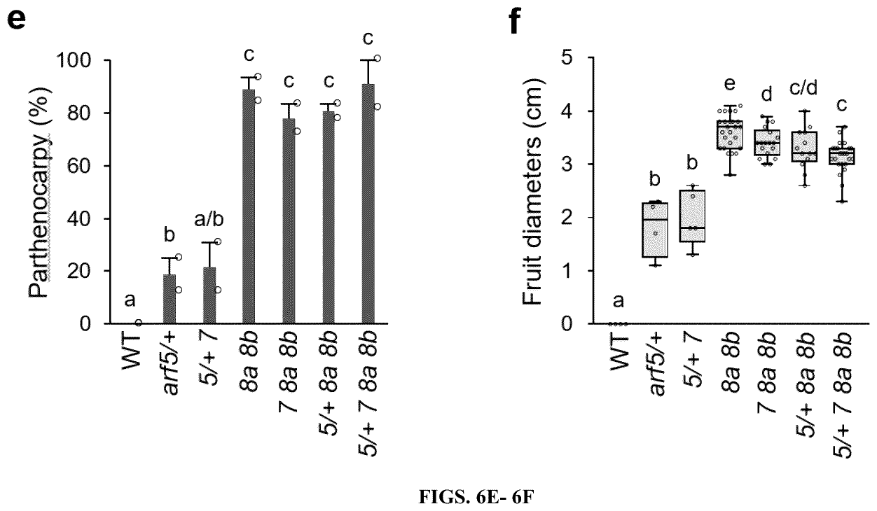

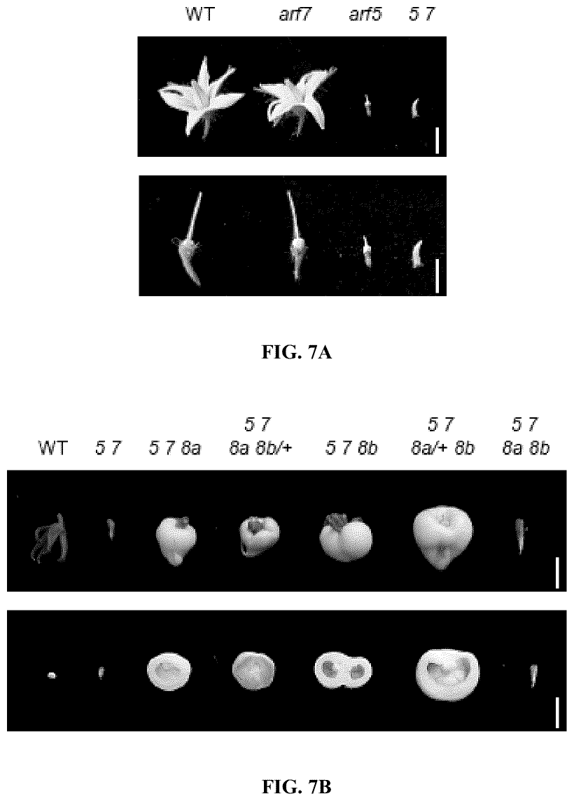

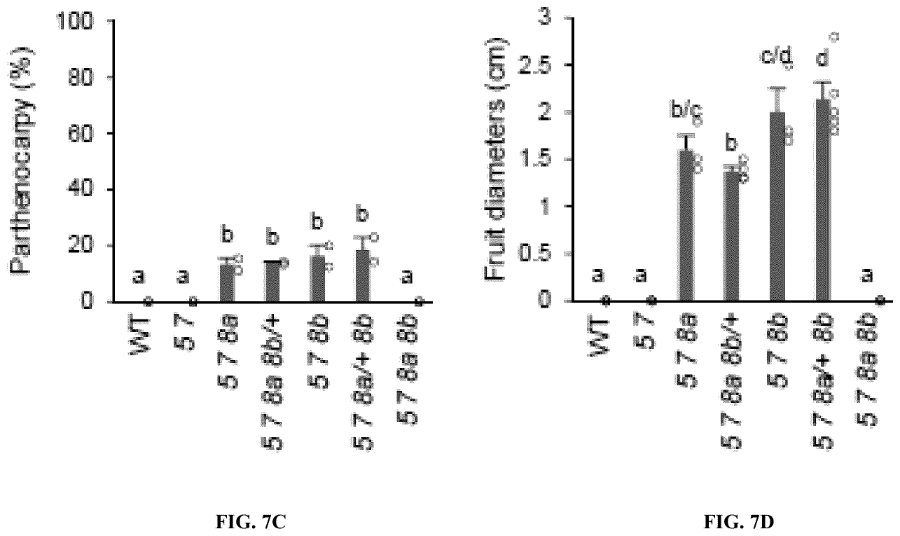

The arf8a arf8b double mutant produced large parthenocarpic fruits, which are likely promoted by other class A-ARFs, such as SlARF5 and SlARF7 that are also expressed at elevated levels in the ovary around anthesis (Hu et al.). To test this idea, higher order arf mutants were generated and characterized. In this study, all single and higher order arf mutants are homozygous, except when specified to be heterozygous. The arf5-1 (arf5) and CR-arf7 (arf7) null alleles were originally generated in the M82 cultivar, whereas the arf8a arf8b mutants are in the MM cultivar. Therefore, the arf5 and arf7 null alleles were introgressed into MM cultivar by genetic crossing 5 times and then these mutants were crossed with 8a 8b to make higher order arf mutant combinations. arf5 displayed severe flower development defects in all floral organs. The arf5 flower is missing petals and anthers, and only consists of tiny ovary, sometimes with sepals (FIG. 6a, FIG. 7a). As a result, arf5 could produce neither normal fruit nor parthenocarpic fruit. The heterozygous arf5/+ mutant showed overall WT-like phenotypes. However, the heterozygous arf5/+ mutant produced small parthenocarpic fruits at a low frequency in comparison to those of 8a 8b (FIG. 6d-6f). The arf7 mutant showed WT-like phenotypes throughout vegetative and reproductive growth. Similar to arf5/+, arf7 produced small parthenocarpic fruits at a low frequency (FIG. 6a-6c). Both 5 8a 8b and 7 8a 8b triple homozygous mutants showed lower parthenocarpy frequency and produced smaller seedless fruits than those of 8a 8b (FIG. 6a-6c). The placenta growth defects in 5 8a 8b and 7 8a 8b were the same as in 8a 8b. These results indicated that SlARF5 and SlARF7 also function in repressing fruit initiation and promoting pericarp expansion during fruit growth, while they are not required for placenta growth.

To examine if SlARF8A and SlARF8B promote pericarp growth in addition to their strong roles in repressing fruit initiation, the effect of adding 8-1 and 8b-1 alleles into the arf5 arf7 background was analyzed. Comparing to the arf5 single mutant, the arf arf7 double homozygous mutant showed even more severe floral defects as its flowers only formed a pin-like structure, which could not develop into fruits (FIG. 6a). Remarkably, both 5 7 8a and 5 7 8a 8b/+ mutants that contain two or one copies of WT 8B produced medium-size seedless fruits with 20-30% frequency, whereas the 5 7 8a 8b quadruple homozygous mutant showed no parthenocarpy at all (FIG. 6a-6c). Similarly, both 5 7 8b and 5 7 8a/+8b mutants that contain two or one copies of WT 8A produced medium-size seedless fruits with ˜15% frequency (FIG. 7b-7d). These results indicated that both SlARF8A and 8B also promote fruit growth. These data revealed a biphasic response in parthenocarpic fruit expansion, which increased initially with removal of 1-2 SlARFs, then decreased when more SlARFs were knocked out, until no growth occurred when all four SlARFs were knocked out. The roles of these SlARFs were verified further using arf mutant combinations with arf5/+ for the parthenocarpy assay, because the homozygous arf5 mutant had severe ovary defect (during flower development) that may interfere with fruit development. The 5/+7 mutant displayed weak parthenocarpy as in arf5/+, again indicating their minor roles in repressing fruit initiation (FIG. 6d-6f). Similar to 7 8a 8b, 5/+8a 8b produced smaller parthenocarpic fruits than 8a 8b, demonstrating that removing one functional copy of SlARF5 in 8a 8b could reduce fruit growth. Furthermore, 5/+7 8a 8b seedless fruits were even smaller than those in 5/+8a 8b and 7 8a 8b (FIG. 6d-6f). Again, the placenta growth in these triple and quadruple mutants remained the same as in 8a 8b.

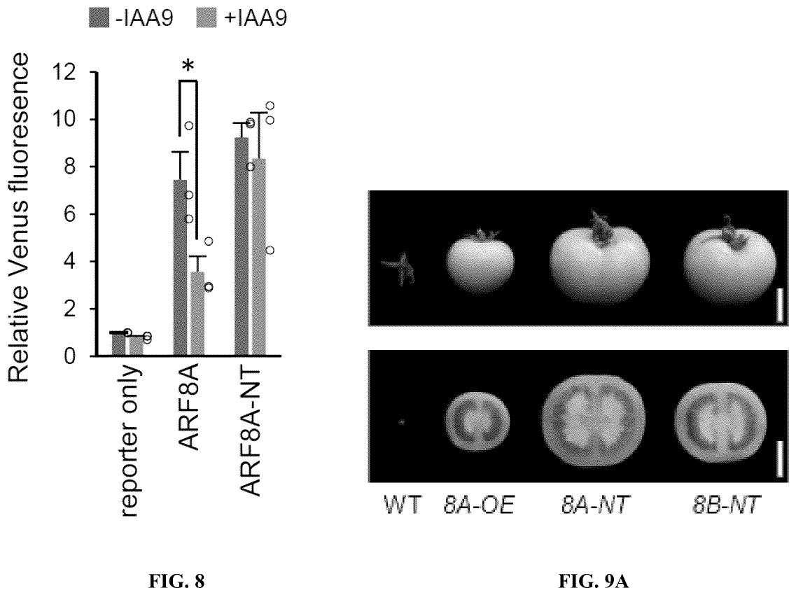

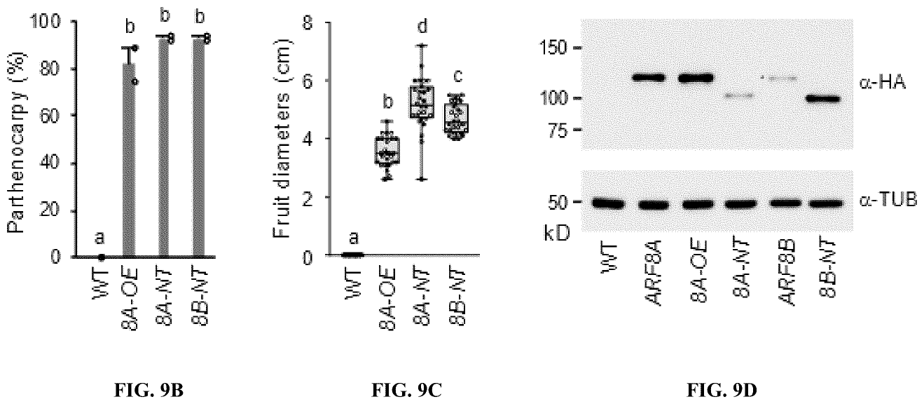

To verify further the role of SlARF8A and 8B in promoting fruit growth, transgenic tomato lines P_{ARF8A}:3F2H-ARF8A-NT and P_{ARF8B}:3F2H-ARF8B-NT, which expressed either truncated SlARF8A or SlARF8B proteins lacking the C-terminal PB1 domain (1-716 a.a. for ARF8A-NT and 1-715 a.a. for ARF8B-NT) were generated. Because the PB1 domain is required for ARF8-IAA9 interaction (Hu et al.), expression of the SlARF8-NT proteins should lead to constitutive auxin signaling by uncoupling SlARF8A/8B activity from SlIAA9. The effect of IAAon ARF8A vs ARF8A-NT was first tested in a synthetic yeast system, which was modified from the auxin response circuit (ARC) that was previously proven functional for testing Arabidopsis auxin signaling components (Pierre-Jerome et al.). In this yeast ARC system, SlARF8A or SlARF8A-NT was expressed in the presence or absence of SlTPL1^{N100}-SlIAA9 fusion (a truncated TPL-IAA9 fusion). The yeast strain also contains an auxin-responsive promoter P3_2x:Venus (an output reporter). Both SlARF8A and SlARF8A-NT induced P3_2x:Venus expression (FIG. 8). However, co-expression of SlTPL1^{N100}-SlIAA9 dramatically down-regulated SlARF8A-induced P3_2x:Venus, but had no effect on SlARF8A-NT activity. Consistent with the yeast ARC results, both transgenic ARF8A-NT and ARF8B-NT tomato lines with high expression levels exhibited strong parthenocarpy (FIG. 9a-9d). Moreover, these ARF8-NT lines produced large seedless fruits with well-developed placenta and locular tissue, similar to the entire (sliaa9) seedless fruit (FIG. 1i) and pollinated WT fruit (FIG. 2a). In contrast, most of the 31 P_{ARF8A}:3F2H-ARF8A transgenic lines produced WT-like seeded fruits (FIG. 8a-8b), except one line with highest expression (referred to as ARF8A-OE displayed obligate parthenocarpy (FIG. 9a-9d). This ARF8A-OE line produced parthenocarpic fruits that are smaller than those of 8A-NT and 8B-NT lines, although its ARF8A protein levels were higher than the ARF8-NT lines. These results support that deletion of the PB1 domain in ARF8A and ARF8B enabled them to promote parthenocarpic fruit growth more effectively than the full-length ARF8.

Altogether, the results from the different arf combinations and the ARF8-NT transgenic lines strongly support that SlARF5, SlARF7 , SlARF8A and SlARF8B all play dual function in fruit development: they act as inhibitors when associated with SlIAA9 during fruit initiation and as activators in fruit growth. SlARF8A and SlARF8B are stronger repressors than ARF5 and ARF7 in fruit initiation, while all four ARFs act as activators to promote pericarp growth. On the other hand, placenta growth is mostly promoted by SlARF8A/8B.

### 3. Spatiotemporal Expression Patterns of ARFs and IAA9 in Ovary

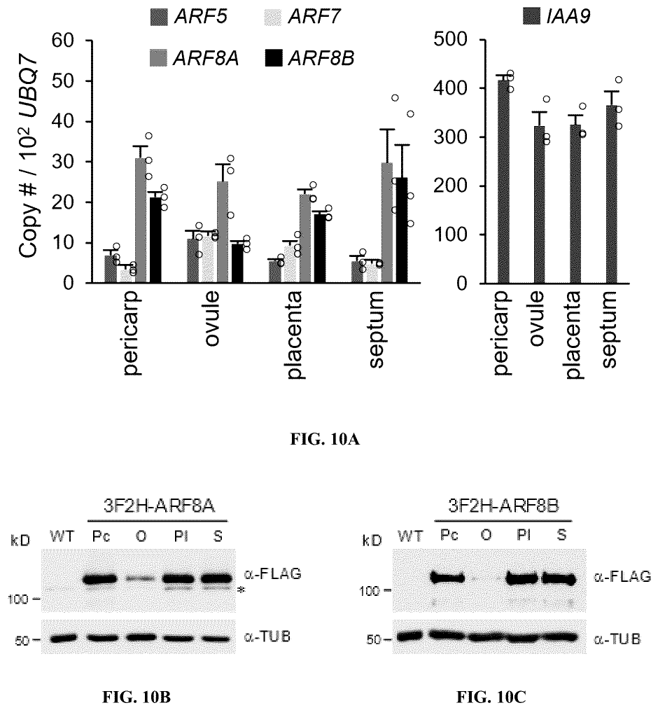

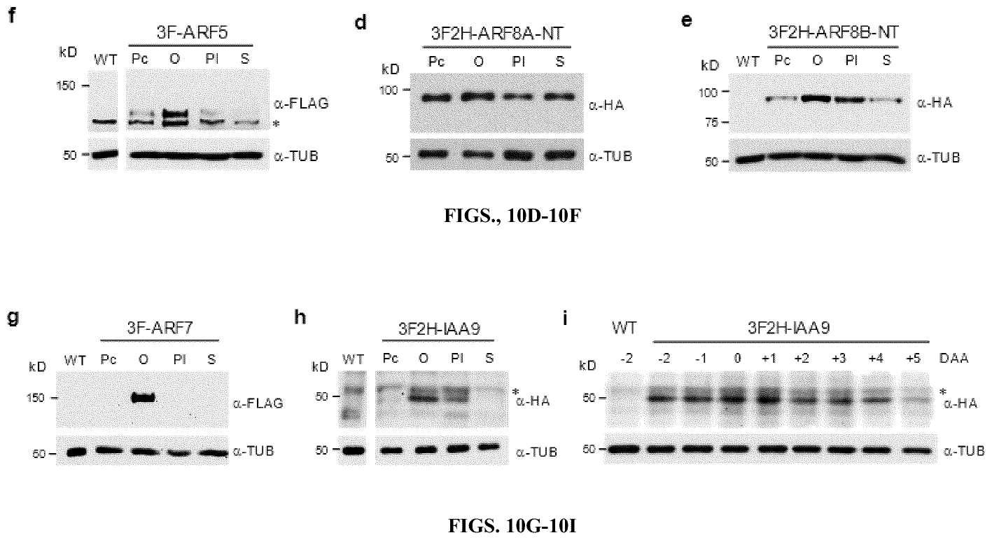

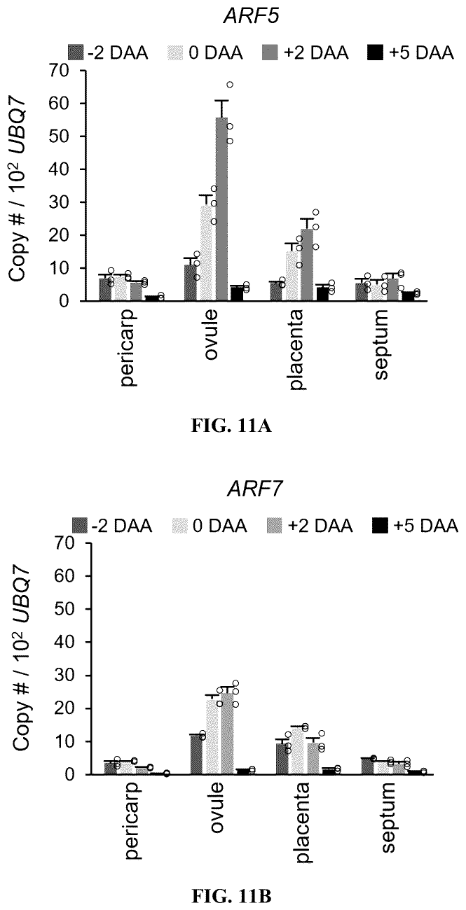

To investigate the roles of these four class A-SlARFs and SlIAA9 in different ovary tissues (pericarp, ovule, placenta and septum) during fruit initiation and growth, absolute transcript levels of these genes were examined around anthesis by RT-qPCR analysis (FIG. 10a and FIG. 11). In −2 DAA ovary, SlARF8A and SlARF8B transcript levels were similar in all four tissues, whereas SlARF5 and SlARF7 transcript levels were highest in ovule (FIG. 10a). SlIAA9 transcript levels were high in all four ovary tissues (FIG. 10a). In addition to −2 DAA ovary, we also monitored spatial and temporal expression patterns of these genes using 0, +2, +5 DAA ovaries (FIG. 11). In general, the distribution patterns for each SlARF and SlIAA9 were similar as those in −2 DAA ovary. Most notably, expression of SlARF5 and SlARF7 in ovule/seeds peaked around 0 to +2 DAA, and dramatically decreased at +5 DAA, implying their roles during the ovule-to-seed transition period.

To monitor spatial distribution of SlARFs and SlIAA9 proteins, transgenic tomato lines expressing epitope-tagged SlARF/IAA9 constructs under corresponding endogenous promoters (P_{GENE}:tag-ARF or IAA9) were generated. SlARF5 and SlARF7 constructs contained a 3xFLAG (3F) tag while SlARF8A, SlARF8B and SlIAA9 had a 3F2H tag. As described earlier, 3F2H-ARF8A and 3F2H-ARF8B are functional in planta to rescue the 8a-1 and 8b-1 mutant phenotypes, respectively (FIG. 3a-3e). 3F-ARF5 and 3F2H-SlIAA9 also rescued all defects in the arf5 homozygous mutant and the sliaa9(entire) mutant, respectively (FIG. 3f-3i). ARF protein localizations were analyzed in four ovary tissues from −2 DAA flower: pericarp, ovule, placenta and septum. Immunoblot analysis indicated that SlARF8A and SlARF8B showed similar spatial expression patterns with high levels in pericarp, placenta and septum, and low levels in the ovule (FIG. 10b-10c). These protein expression patterns are mostly consistent with their transcript levels, except in the ovule, possibly due to downregulation of SlARF8A/8B translation by miR167 in ovule³³. Indeed, SlARF8A-NT/8B-NT that lack the miR167 target sequence within the PB1 domain showed similar levels in all four ovary tissues (FIG. 10d-10e). SlARF5 and SlARF7 showed opposite expression patterns to those of SlARF8A and SlARF8B in that they were mainly present in ovules (FIG. 10f-10g), although low levels of SlARF5 were also detected in the pericarp, septum and placenta (FIG. 10f). SlIAA9 was detected in all four tissues: high levels in ovules, medium levels in placenta and low in pericarp and septum (FIG. 10h), again showing discrepancy between transcript and protein distributions for this key auxin signaling component. SlIAA9 protein levels were also examined in the ovary daily from −2 DAA to +5DAA, and found that it was slightly reduced at +2 DAA and was significantly decreased at +5 DAA (FIG. 10i), consistent with the idea that auxin produced after anthesis results in degradation of IAA9 protein.

Taken together, the distinct spatial distributions of SlARFs and SlIAA9 proteins in ovary indicated specific roles of SlARFs and SlIAA9/ARF repressor modules in regulating development of different fruit tissues, and these spatial results were, in general, consistent with arf and entire mutant phenotypes.

### 4. SlARF8A/8B Target Genes During Fruit Initiation and Growth

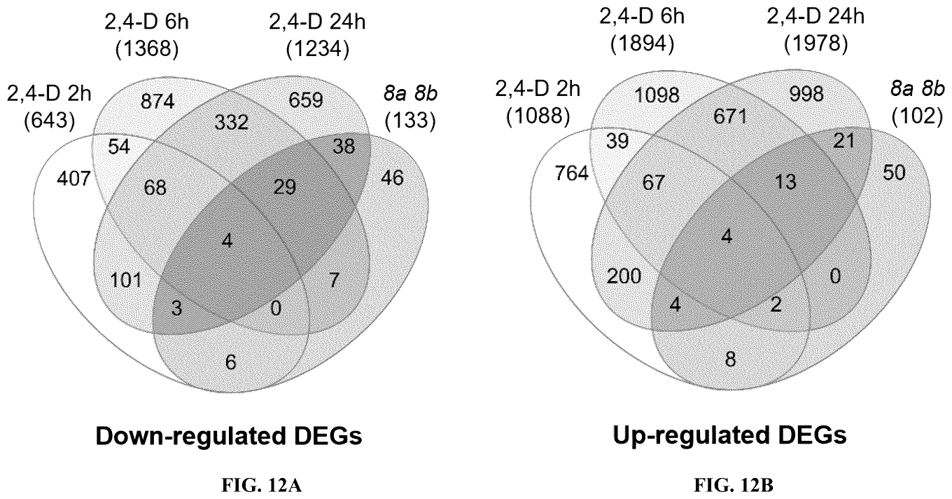

To identify putative SlARF8A/8B target genes that are involved in auxin-mediated fruit set and growth, transcriptome analysis was performed by RNA-Seq using the following samples: (1) −2 DAA ovaries of 8a 8b and WT; (2) −2 DAA WT ovaries treated with mock or 100 μM 2,4-D (a synthetic auxin) for 2, 6 or 24 h, respectively. These 2,4-D treatment time points were chosen based on previous RT-qPCR results indicating clear changes in expression of most auxin-responsive genes at 6 h, although some genes only showed significant changes at 24 h. A 2 h treatment was also included in an attempt to identify early auxin response genes. Three biological repeats were included in each set of samples, except that the +2,4-D 2 h treatment used two biological repeats for RNA-seq analysis. The differentially expressed gene (DEG) lists for SlARF8A/8B-responsive genes (235) and for auxin-responsive genes (1731/3262/3212 in 2/6/24 h treatments) were identified with the following parameters: fold change >1.5; p<0.1 for 8a 8b vs. WT dataset; p<0.05 for +2,4-D vs. mock treatment dataset. By comparing among these gene lists, we found that almost 60% of DEGs that were responsive to 8a 8b (139 DEGs out of 235 total) were co-regulated with at least one of the +2,4-D time points (FIG. 12a-12b). Furthermore, most of the co-regulated genes were found in the +2,4-D 6 h treatment and/or 24 h treatment dataset (108 DEGs out of 139 total, data not shown).

For all co-regulated DEGs, Gene Ontology (GO) analysis was performed to identified 75 enriched GO terms that can be organized into 16 groups of different biological processes, cellular components and molecular functions. Although the co-regulated DEGs belong to a variety of biological pathways, certain groups were more representative, including transcription factors, hormone-related genes and growth-related genes (Table 4).

TABLE 4
Selected gene groups from co-regulated
genes between 8a 8b and +2,4-D 6 h/24 h.

| Transcrption Factors | Hormones | Growth-Related |
|---|---|---|
| Down-Regulated: | Down-Regulated: | Up-Regulated: |
| AG1, AGL6, MADS2 | ACO4 | XTH7, XTH15 |
| HOX.1 | Up-Regulated: | NR |
| MYB21 | ERF4 | TPX1 |
| HSF | ABAH | SUS |
| Up-Regulated: | GA20ox1 | MIPS |
| HD52, ZF-HD1 | CKX2 | |
| ERF4 | | |
| MYB77 | | |

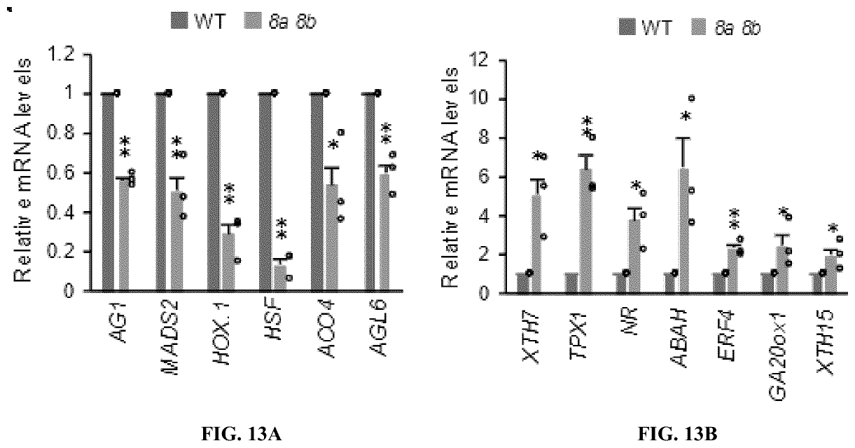

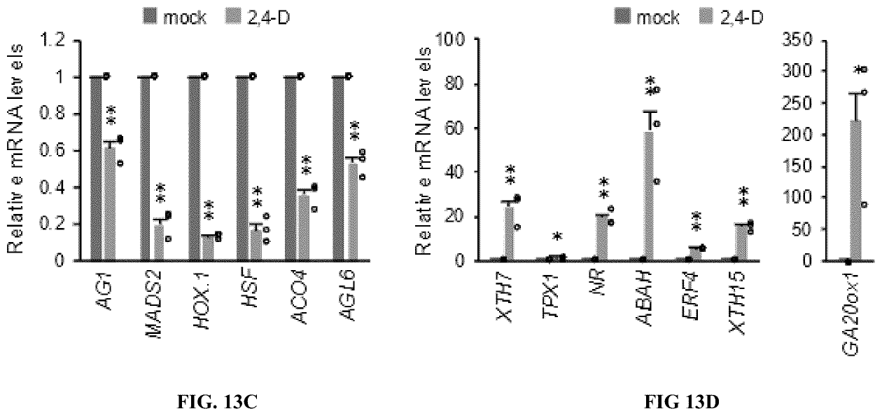

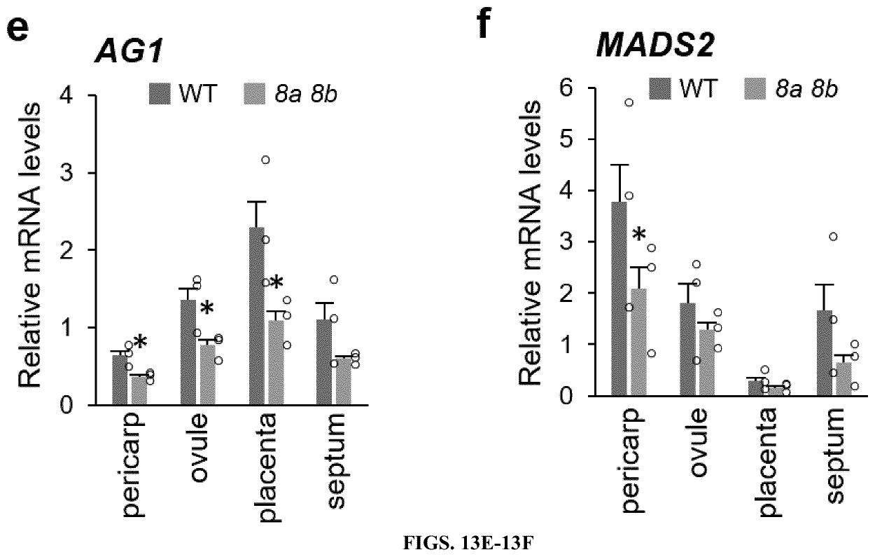

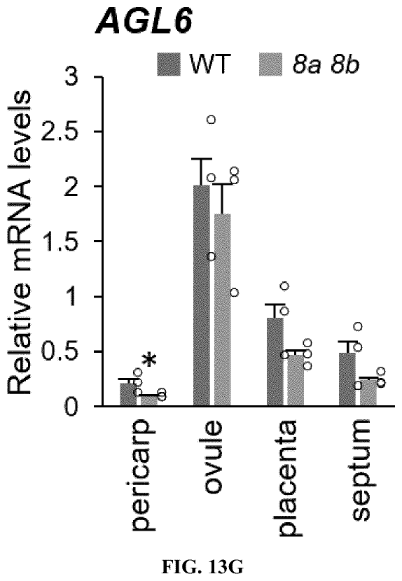

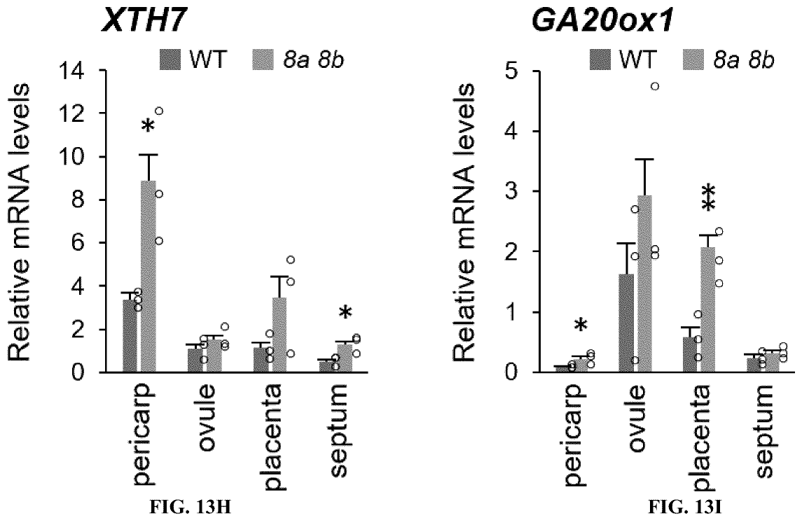

Eleven genes were chosen from these 3 groups to verify their expressions in 8a 8b by RT-qPCR. These include four genes encoding transcription factors [two MADS-BOX genes, AGAMOUS1 (AG1) and MADS-BOX PROTEIN 2 (MADS2), HOMEOBOX-LEUCINE ZIPPER PROTEIN 1 (HOX.1) and HEAT STRESS TRANSCRIPTION FACTOR (HSF)]; four that function in hormone metabolism and signaling [ACC OXIDASE 4 (ACO4), ABA 8′-HYDROXYLASE (ABAH), ETHYLENE RESPONSE FACTOR 4 (ERF4) and GA 20-OXIDASE 1 (GA20ox1)]; two genes related to cell wall modification [Xyloglucan endotransglucosylase/hydrolase 7 (XTH7) and Peroxidase1 (TPX1)]; and a Nitrate Reductase (NR) for converting inorganic nitrate to organic nitrite which is crucial for protein production in plants. Consistent with the RNA-Seq data, the RT-qPCR assays confirmed that expression of these genes was down- or up-regulated in 8a 8b (FIG. 13a, 13b) or by 2,4-D treatment (FIG. 13c, 13d). Several 2,4-D-responsive growth-related genes were further tested by RT-qPCR, and it was found that XTH15 was also induced in 8a 8b ovary (FIG. 13b). The altered expression of hormone-related genes suggested that 8a 8b has reduced ethylene and ABA biosynthesis and signaling (by downregulating ethylene biosynthesis gene ACO4, upregulating ERF4 whose ortholog in Arabidopsis was shown to inhibit ethylene and ABA responses, and upregulating ABAH for ABA deactivation), and increased GA levels (by upregulating GA20ox1). Because fruit initiation is known to be repressed by ethylene and ABA, and promoted by GA, the observed changes in hormone potentials would facilitate fruit initiation in the 8a 8b ovary. Likewise, XTH7, XTH15 and TPX1 function in promoting cell wall biosynthesis, their up-regulation in 8a 8b ovary points to increased potential for fruit growth.

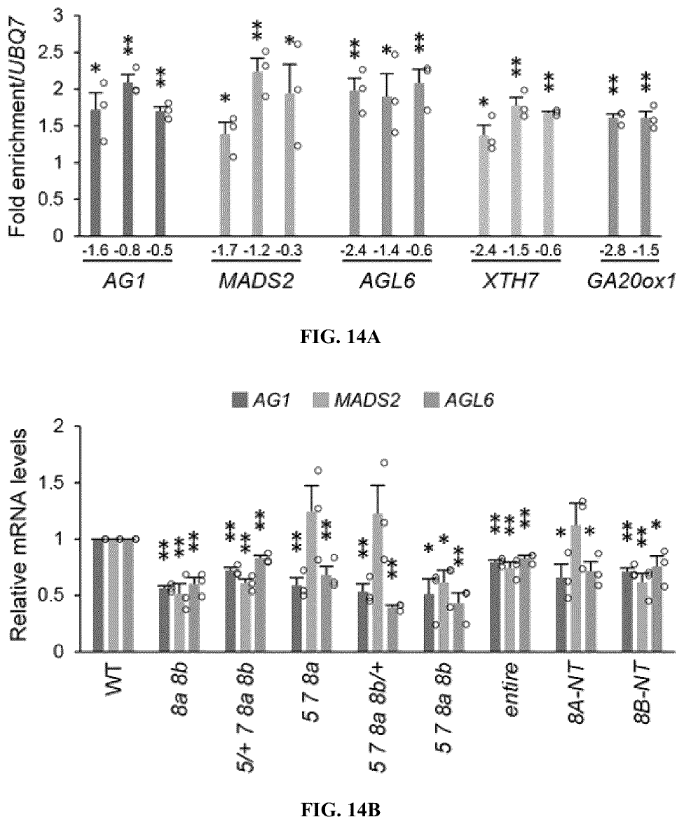

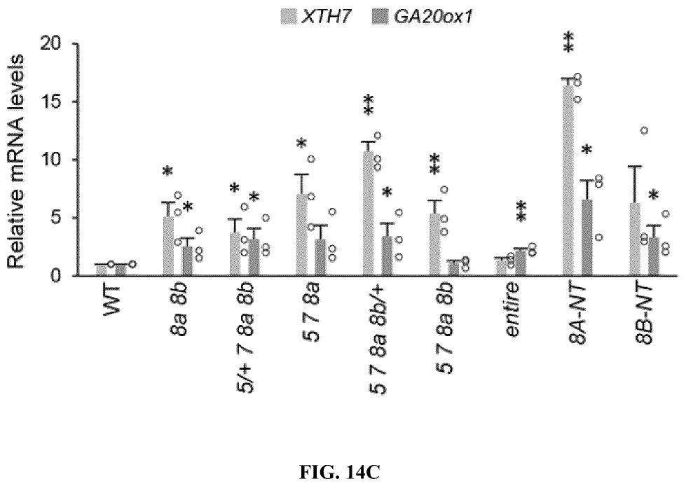

Interestingly, two of the down-regulated transcription factors in 8a 8b are MADS-BOX genes AG1 and MADS2. Several MADS-BOX genes [e.g., AG1, AGL6 (AGAMOUS-LIKE 6), TM29] have been implicated as repressors of fruit initiation as their transcript levels were significantly down-regulated in ovary upon pollination or by auxin treatment. More importantly, the AGL6 null mutants display strong parthenocarpy phenotype, indicating that AGL6 is a major repressor in fruit set. RT-qPCR analysis confirmed that AGL6 transcript levels were repressed in 8a 8b ovary (FIG. 13a). Because SlARF8A and SlARF8B proteins in the −2DAA ovary are more abundant in most tissues except in the ovule, the MADS-BOX, XTH7 and GA20ox1 transcript levels were further analyzed to determine whether altered transcript distributions correlate with the spatial localization of SlARF8A/8B proteins. AG1, MADS2, AGL6 all showed reduced transcript levels in pericarp of 8a 8b, comparing to the corresponding tissues in WT (FIG. 13e-13g). In placenta and septum, these genes also showed reduced expressions in 8a 8b, although only AG1 had significant difference from WT. In contrast, only AG1 showed reduced expression in 8a 8b ovule in comparison to that in WT, while expression of MADS2 and AGL6 did not change significantly (FIG. 13e-13g), indicating that downregulation of these MADS-BOX genes in 8a 8b coincides with SlARF8A/8B protein localization in ovary. Similarly, expression of XTH7 and GA20ox1 was induced in pericarp/septum and pericarp/placenta tissues of 8a 8b respectively, while not in ovule (FIG. 13h-13i). These results suggest that the MADS-box genes, XTH7 and GA20ox1 are SlARF8A/8B targets. To test if SlARF8A/8B directly regulate these genes, ChIP-qPCR assay was performed using −2 DAA ovaries of the P_{ARF8A}:3F2H-ARF8A line. Cross-linked chromatin was immunoprecipitated using anti-FLAG beads, and qPCR was performed using 2-3 primer pairs that span the promoter region of each MADS-box gene, XTH7 and GA20ox1. On average, 1.7- to 2.3-fold enrichment in these promoters when normalized against UBQ7 promoter (FIG. 14a), supporting that SlARF8A directly binds to these target promoters to regulate their expression.

### 5. MADS-BOX Expression in Unpollinated Ovary Requires IAA9/ARFs

RNA-Seq and ChIP-qPCR results showed that the three MADS-BOX genes (AG1, MADS2, AGL6) that are downregulated in fruit initiation are direct targets of SlARF8A/8B. Repression of these MADS-BOX genes appeared to be useful biomarkers to assess whether phase transition from ovary to fruit set had occurred, especially when no further growth was observed (e.g., the 5 7 8a 8b mutant). RT-qPCR analysis indicated that −2 DAA ovaries of the quadruple 5 7 8a 8b mutant showed reduced expression of all three MADS-BOX genes, similar to those in the parthenocarpy mutants 8a 8b, 5/+7 8a 8b, 5 7 8a and 5 7 8a 8b/+ (FIG. 14b). The only exception was that MADS2 expression was not reduced in 5 7 8a and 5 7 8a 8b/+ comparing to WT, possibly due to the presence of SlARF8B. These results support that fruit initiation had occurred in these mutants, although the quadruple 5 7 8a 8b mutant ceased in subsequent fruit growth process. Expression of growth-related genes in the higher-order arf mutants was examined. Consistent with the mutant phenotypes, GA20ox1 showed increased expression in all parthenocarpy arf mutants, but was not elevated in 5 7 8a and 5 7 8a 8b mutants comparing to that in WT (FIG. 14c). In contrast, XTH7 expression was induced in all higher-order arf mutants, including 5 7 8a 8b (FIG. 14c), suggesting that induction of this gene is insufficient to trigger fruit growth. Likewise, in −2 DAA ovary of SlARF8A-NT and SlARF8B-NT lines, the MADS-BOX genes were all repressed comparing to WT, except for MADS2 in 8A-NT (FIG. 14b). Both XTH7 and GA20ox1 were induced in these ARF8-NT lines except for XTH7 in (FIG. 14c). In addition, the three MADS-BOX genes and GA20ox1 were also repressed (FIG. 14b) or induced (FIG. 14c), respectively, in −2 DAA ovary of the entire (iaa9) mutant comparing to WT. These results suggested that IAA9 also participates in the regulation of these genes, and that increased expression of these MADS-BOX genes in unpollinated WT ovary requires IAA9 and ARFs.

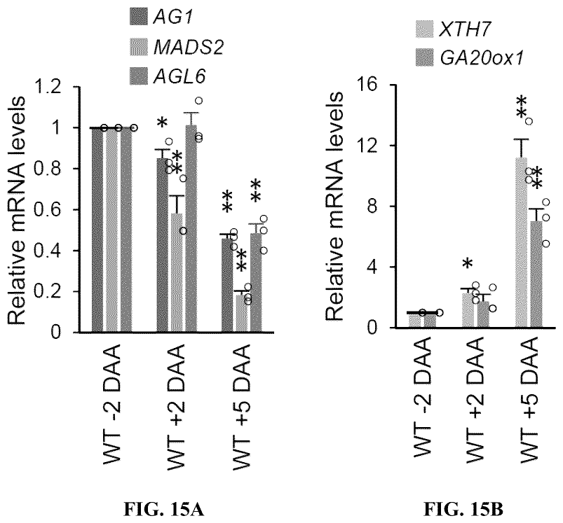

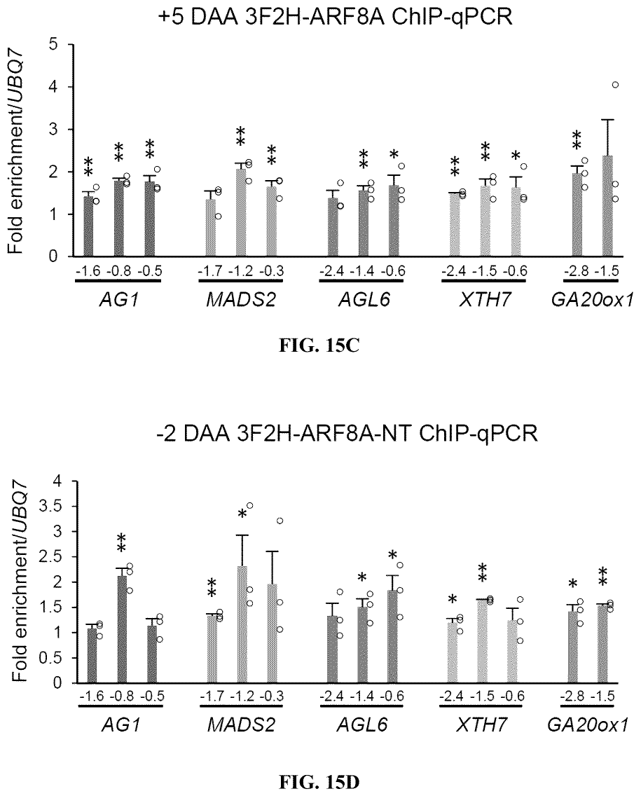

Current dual function model of class A-ARFs suggests that ARFs bind to their target promoters both in the presence and absence of IAAs. Our yeast ARC results shown earlier (FIG. 8) is consistent with this model as both ARF8A and ARF8A-NT (without the PB1 domain) activated transcription of the synthetic auxin-responsive promoter P3_2x. To further test this in planta, RT-qPCR analysis was performed using +2 and +5 DAA WT fruits when the IAA9 protein levels were slightly or significantly reduced, respectively (FIG. 10i). In comparison to −2 DAA ovary, expression of five selected ARF8A target genes in +2 DAA fruits only displayed subtle changes (AG1, MADS2, and XTH7) or were unaltered (AGL6 and GA20ox1 ), consistent with the slight reduction of IAA9 protein amount at this time point. As expected, in +5 DAA fruits, all three MADS-BOX genes were repressed 2-5 fold, while XTH7 and GA20ox1 genes were induced 11- and 6-fold, respectively (FIG. 15a-15b). More importantly, ChIP-qPCR analysis confirmed that ARF8A still bound to these target promoters in +5 DAA fruit where IAA9 was mostly degraded (FIG. 15c). ChIP-qPCR using the ARF8A-NT line also showed binding of ARF8A-NT to the same ARF8 target promoters in −2 DAA ovary, further confirming that ARF8A can bind to these selected targets with or without forming complex with IAA9 (FIG. 15d).

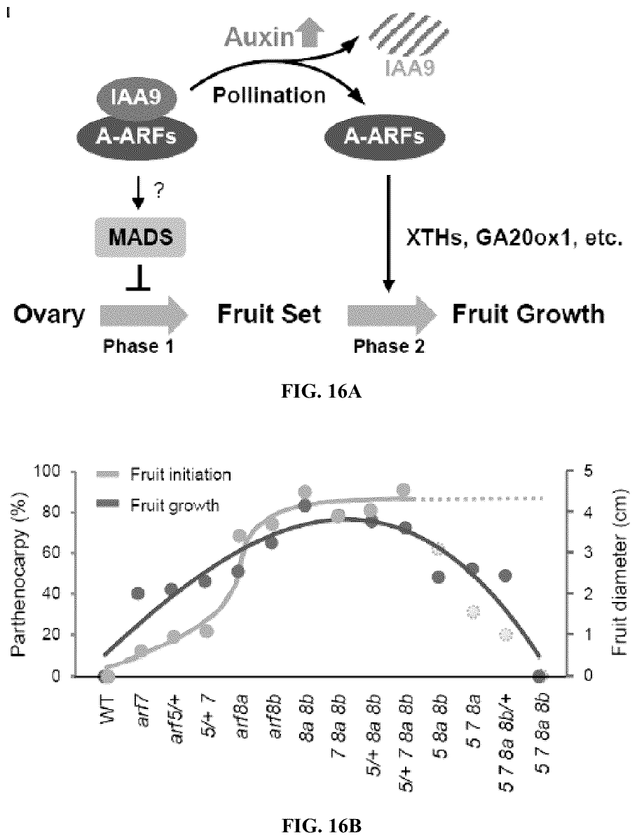

These studies illustrated the dual function of four class A-SlARFs (ARF5, ARF7, ARF8A, ARF8B) in inhibiting fruit initiation while promoting fruit growth in tomato (FIG. 16a). Importantly, altering doses of these four class-A SlARFs led to biphasic fruit growth responses as summarized in FIG. 16b, revealing the complex combinatorial effects of multiple ARFs in fine-tuning auxin-mediated fruit set and subsequent fruit growth. Generation of the higher order slarf mutant combinations and the identification of marker genes for fruit initiation are critical tools, which allowed dissection of the separate roles of ARFs in fruit set and fruit growth. By doing so, the activator function of these class A-SlARFs was unmasked in fruit growth, which was impossible in previous studies because of their inhibitory role in fruit set. SlARF8A and SlARF8B are central repressors (together with SlIAA9 ) in fruit set as 8a 8b led to maximum parthenocarpy frequency, although SlARF5 and SlARF7 also play minor repressive roles in fruit set as arf5/+ and arf7 mutants showed low rates of parthenocarpy. The parthenocarpy frequency, defined as percentage of fruits (diameter ≥1 cm) that developed from unpollinated ovaries, reflects the combined effects of both fruit set and subsequent growth. Hence, three MADS-BOX genes (AG1, AGL6 and MADS2) were used as molecular markers to gauge the onset of fruit initiation because these genes are direct targets of SlARF8A/8B (FIGS. 12-14) and function as repressors of fruit set. Reduced expression of these MADS-BOX genes suggests fruit initiation has occurred (FIG. 14b), even in the absence of apparent fruit growth in 5 7 8a 8b quadruple mutant (shown as the dashed line in FIG. 16b).

To dissect the individual and combined role of these SlARFs in fruit growth, the effects of these arf mutants in altering fruit sizes was analyzed. It was found that parthenocarpic fruit diameter increased from arf5/+ or arf7 mutants to arf8 single mutants, then to 8a 8b double mutant (FIGS. 6 and 16b). However, further adding arf5, arf5/+ or arf7 mutations to the 8a 8b background resulted in reduced fruit growth. Eventually, fruit growth was completely abolished in the arf5 7 8a 8b quadruple mutant, although fruit initiation is likely activated in this mutant judging by repressed expression of MADS-BOX genes (FIG. 14b, 16b). The strong parthenocarpic phenotype of the ARF8A/8B-NT transgenic lines (expressing truncated ARF8A/8B proteins lacking the PB1 domain) further illustrated the promoting role of ARF8A and ARF8B in fruit growth when uncoupled from IAA9 (FIG. 9a). These results strongly support that all 4 class A-ARFs play a dual role in fruit initiation and fruit growth, and they contribute to fruit growth in a dosage-dependent manner. Before anthesis (pollination), the repressive role of these class A-SlARFs in fruit set is dependent on SlIAA9 as the null sliaa9mutant (entire) and the ARF8-NT lines all displayed strong parthenocarpy. Expression patterns of SlIAA9 and SlARF proteins before anthesis are consistent with their corresponding roles during fruit set. It was found that SlIAA9 protein was present in the whole ovary. SlARF8A and SlARF8B proteins were mainly localized to pericarp, septum and placenta, so knocking out these SlARFs removed their repression in these tissues. In contrast, SlARF5 and SlARF7 proteins were mainly localized in ovules so that arf5/+ or arf7 mutant only displayed weak parthenocarpy because of the presence of the strong repressors SlARF8A/8B in other tissues. Previous studies showed that AGL6 and possibly additional MADS-BOX genes are key repressors of fruit set. Transcript levels of these genes are down-regulated by silencing or null mutation of IAA9, which was confirmed in the studies described herein (FIG. 14b). Importantly, it was found that these MADS-BOX genes were also down-regulated in the 8a 8b mutant (FIG. 13a), and they are direct targets of SlARF8A by ChIP-qPCR (FIG. 14a). In addition, expression of these MADS-BOX genes was downregulated at +5 DAA WT ovary (when IAA9 protein levels were low) in comparison to that at −2DAA (when IAA9 levels were high) (FIGS. 15a and 15b, FIG. 10i). Taken together, these results strongly support that IAA9/ARF complexes directly promote transcription of MADS-BOX genes (AG1, MADS2 and AGL6) before anthesis (FIG. 16a). This is a surprising finding as IAAs are only known as transcriptional repressors (Lavy et al. Development 143, 3226-3229, (2016). It is possible that the reduction in the IAA9 protein levels after pollination would lead to reduced expression of the MADS-BOX genes to basal levels in the absence of the IAA9/ARF complexes. Alternatively, IAA9/ARFs may inhibit unidentified repressor(s) of MADS-BOX genes before anthesis, and ARFs may promote expression of repressor(s) of MADS-BOX genes after pollination when IAA9 levels are reduced.

The ARF8A/8B and auxin co-regulated DEG list highly correlates with a recent study on tomato transcriptomic reprogramming after pollination or auxin treatment (Hu et al. Histone posttranslational modifications rather than DNA methylation underlie gene reprogramming in pollination-dependent and pollination-independent fruit set in tomato. New Phytol 229, 902-919,(2021)). Among the139 DEGs studied, 96 and 91 genes overlap with the 4-day after pollination DEG list (4DPA) and the 4-day after IAA treatment DEGs (4IAA) in that study, respectively. For the 13 genes tested in FIG. 13a-13d, 10 of them showed correlation with 4DPA DEGs and 9 with 4IAA DEGs in the previous study, including AG1, MADS2, XTH7 and GA20ox1. Interestingly, the same study also identified a number of epigenetic regulators on their 4DPA/4IAA DEG list, nine of which were on the 24 h 2,4-D DEG list studied (E3 ubiquitin-protein ligase UHRF1 (VIM1-like), Histone-lysine N-methyltransferase, H3 lysine-9 specific SUVH4 (SISDG5), cytosine-5 DNA methyltransferase1L (SlCMT3-like1), Nucleosome assembly protein 1-like protein 2 (NAP1;2), cytosine-5 DNA methyltransferase3L (SlCMT3-like2), Histone-binding protein, RBBP7 (MSI4), ATP-dependent DNA helicase DDM1b (SlDDM1b), ATP-dependent DNA helicase DDM1a (SlDDM1a), Histone-lysine N-methyltransferase MEDEA (EZ2)). Their study further revealed strong correlation between changes in transcription levels and alterations in H3K9ac or H3K4me3 histone marks at target genes during pollination and IAA-induced fruit set. These results point to synergistic regulation of histone modification and gene expression by active auxin signaling at fruit set. See tables with data in Hu, J. et al., Nature Plants, 2023 May; 9(5):706-719 (doi: 10.1038/s41477-023-01396-y), which is incorporated in this disclosure for all purposes as if fully set forth herein.

Interestingly, the lack of placenta development of 8a 8b double mutant is completely opposite to its whole fruit growth phenotype. The large parthenocarpic fruit of 8a 8b with very little placenta growth suggests that regulation of fruit growth could be divided into two compartments: the growth of fruit wall, i.e. pericarp and septum, and the growth of inner tissue, i.e. seeds and placenta. In the 8a 8b mutant, fruit growth is achieved solely by the growth of fruit wall. In contrast, the entire (iaa9) mutant produced large parthenocarpic fruits (from emasculated flowers), which resemble WT fruits (from self-pollinated flowers) with well-developed placenta and locular tissue, except seedless. However, entire could not rescue the placenta defects in 8a 8b. In addition, the ARF8A/8B-NT transgenic lines produced large parthenocarpic fruits with similarly well-developed placenta and locular tissue, confirming that placenta growth is exclusively promoted by SlARF8A and SlARF8B, which is different from the involvement of all four ARFs in promoting pericarp and septum growth.

By analyzing high order mutants of four A-SlARFs, the studies described herein uncovered their activator role in mediating auxin-induced fruit growth despite their inhibitory role in fruit initiation. This work also demonstrated that growth of different tissues of tomato fruit is controlled by different combinations of SlARFs, which is potentially useful for agronomic applications in controlling growth of specific fruit tissues. In addition, the high parthenocarpy frequency of the 8a 8b mutant led to higher fruit yield than that of WT. Given the globally changing climate, the knowledge on the molecular mechanism of class A-SlARFs-controlled parthenocarpic fruit development could provide valuable tools to generate resilient crops with enhanced fruit yield to increase food security.

### EXAMPLE II. SlARFSA CRISPR Mutants With PB1 Domain Truncations Produce Seedless Fruits

As described above, the transcription factor SlARF8A plays a key role in regulating fruit initiation and fruit growth in tomato. Expression of a truncated SlARF8A that lacks the C-terminal PB1 domain under its own promoter (P_{ARF8A}:SlARF8A-NT) in transgenic tomato plants led to the production of large seedless fruits (MoneyMaker cultivar). Importantly, the P_{ARF8A}:SlARF8A-NT plants look similar to wild type (WT) plants during the vegetative stage, including their growth rate, stem height and overall plant stature. Their flower morphology is also comparable to WT. Based on this data, CRISPR/Cas9 technology was used to make similar truncation mutations in the endogenous SlARF8A locus to create seedless tomato lines.

### A. Creating Seedless Tomatoes Using Gene Editing Technology

### 1. Plant Materials, Growth Conditions and Plant Transformation

Tomato (Solanum lycopersicum) cultivar Moneymaker (MM) was used as WT in this study. Tomato plants were grown under normal condition in the greenhouse at Duke University with 16 h day/8 h night light cycle. The light intensity at plant canopy level was 550 μmol/m²/sec. Temperatures in the greenhouse were maintained at 25° C./20° C. (day/night), and humidity was maintained between 30-40% RH. The heat stress test was conducted in a climate-control chamber at the Duke Phytotron. The chamber was maintained with the same light cycle, light intensity and humidity as the greenhouse. The temperatures in the chamber for the heat stress test were set to 38° C. (maximum)/28° C. (day/night). The temperature in the chamber was increased gradually from 28° C. at dawn to 38° C. within 6 hr, then kept at 38° C. for 4 hr, then gradually decreased for 6 hr until 28° C. at dusk.

### 2. ARF8A CRISPR/Cas9 Construct

Forward and reverse primers for cloning the SlARF8A gRNA DNA sequences are listed in Table 5. ARF8A gRNA sequences are shown in uppercase.

TABLE 5
gRNA sequences

gRNA1 primers:
5′ - gtcgaagtagtgattGTGTGCCCCGTACATTTGTCAgttttaga
gctagaa (SEQ ID NO: 73)
5′ - ttctagctctaaaacTGACAAATGTACGGGGCACACaatcacta
cttcgac (SEQ ID NO: 74)
gRNA2 primers:
5′ - gtcgaagtagtgattGATGTCCGGACTTCCATCTATgttttaga
gctagaa (SEQ ID NO: 75)
5′- ttctagctctaaaacATAGATGGAAGTCCGGACATCaatcactac
ttcgac (SEQ ID NO: 76)

The SlAR8A gRNA sequences were first separately incorporated into pCR4-TOPO-GFP-gRNA (Peterson et al.,Genome-Wide Assessment of Efficiency and Specificity in CRISPR/Cas9 Mediated Multiple Site Targeting in Arabidopsis. PLoS One 11, e0167169 (2016)) to replace GFP sequence by site-directed mutagenesis, then transferred together to the Cas9-containing binary vector pCUT3 (Peterson et al., 2016) by Gibson Assembly (NEB E2611) to create the ARF8A CRISPR/Cas9 construct (pCUT3-SlARF8A gRNA1/2).

3. Generation of arf8a-CR Mutants

ARF8A CRISPR/Cas9 construct was first introduced into Agrobacterium tumefaciens strain GV3101, which was then used for tomato transformation following a tissue culture transformation method described previously (Bajwa et al., 2013). Transgenic tomato T0 lines were selected using kanamycin. arf8a-CR mutant alleles in T0 lines were amplified by PCR using primers spanning the ARF8A CRISPR gRNA target sites: 5′-GGGCTCCAGTTCACCCTCTCTCTC (SEQ ID NO: 77) and 5′-GTCTACATCACATATGTCCTGATC (SEQ ID NO: 78).

The PCR products were cloned into pCR8/GW/TOPO vector (ThermoFisher), and the molecular lesions in individual clones were identified by DNA sequence analysis. To separate coexisting alleles and remove the Cas9 transgene, a representative T0 mutant line (#2) was backcrossed to WT once. Homozygous arf8a-CR-1 and-CR-2 mutants without the CRISPR gRNA-Cas9 transgenes were identified in the F2 generation by genotyping using dCAPs primers detecting gRNA1 mutation sites.

Genotyping primers for the arf8a-CR-1 allele are 5′-CCTCTATGCCACTAGGGACTTC (SEQ ID NO: 79) and 5′-CCTAAACAACAGAACTAGAACCTTTA (SEQ ID NO: 80). With Msel digestion, PCR product of the arf8a-CR-1 allele would be cut into two DNA fragments of 121 bp and 25 bp, while the WT allele PCR product of 146 bp remained intact.

Genotyping primers for the arf8a-CR-2 allele are 5′-GCACAAACTGTGCCCCGTACCTTT (SEQ ID NO: 81) and 5′-GTCCAATGACCTCCCAAGGGAC (SEQ ID NO: 82). With BslI digestion, PCR product of the arf8a-CR-2 allele would be cut into two DNA fragments of 127 bp and 23 bp, while the WT allele PCR product of 150 bp remained intact.

### 4. Heat Stress Experiment

Tomato plants were first grown under normal conditions for six weeks to reach peak flowering stage. Then the plants were divided into two groups, one group was transferred to heat stress condition while the other group remained in normal condition. After eight weeks, all fruits were collected for weight measurements.

### B. Results

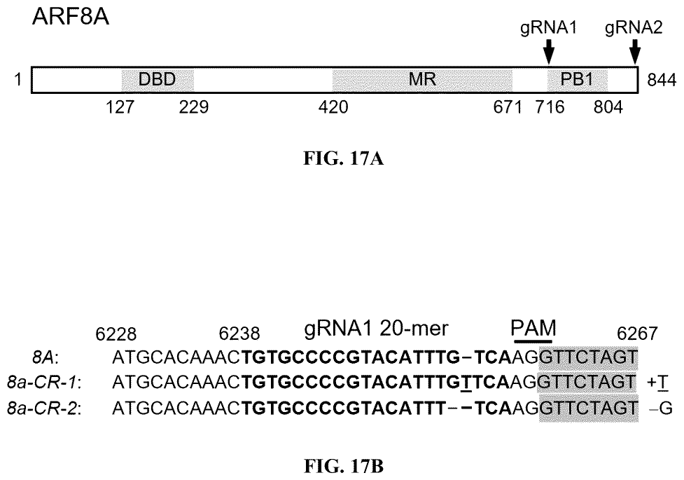

A CRISPR/Cas9 gRNA construct was designed that targets both ends of the PB1 domain encoding sequences in SlARF8A with two gRNAs to generate deletion mutations that result in truncated ARF8A protein lacking the C-terminal PB1 domain (FIG. 17a). The ARF8A CRISPR Cas9 construct was used to generate transgenic tomato lines in the MoneyMaker (MM) cultivar, and six T0 transgenic tomato plants were recovered. DNA sequence analysis of four TO lines (#2, 3, 4, 6), named arf8a-CR mutants, indicated that none of these lines contained a large DNA deletion of the entire PB1 domain. Notably, all four lines had small insertions or deletions that cause frameshift mutations at one or both gRNA target sites within the SlARF8A locus (Table 6, FIG. 17b). Each T0 line contains two different arf8a alleles, all of which would result in deletion of the PB1 domain in the encoded protein due to frameshift mutations at the gRNA1 target site (Table 6). FIG. 17b shows the DNA sequence spanning the ARF8A CRISPR/Cas9 gRNA1 target site in arf8a-CR-1 and arf8a-CR-2 alleles, which coexist in the T0 #2 line.

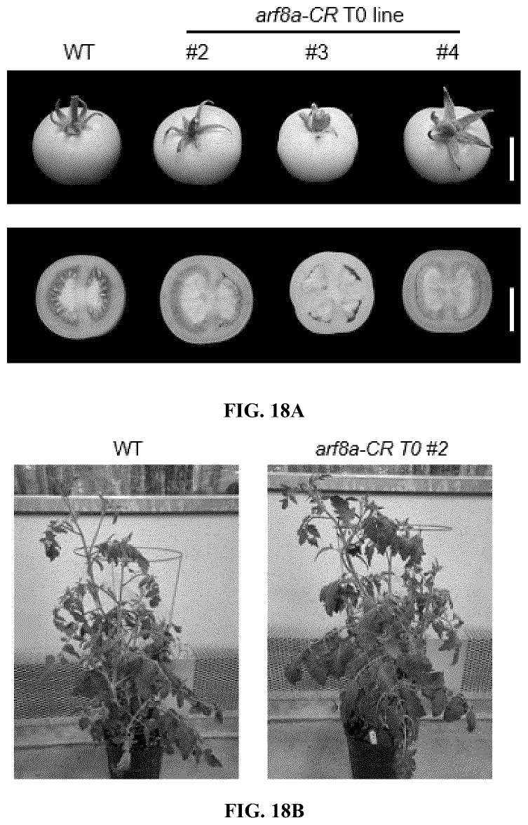

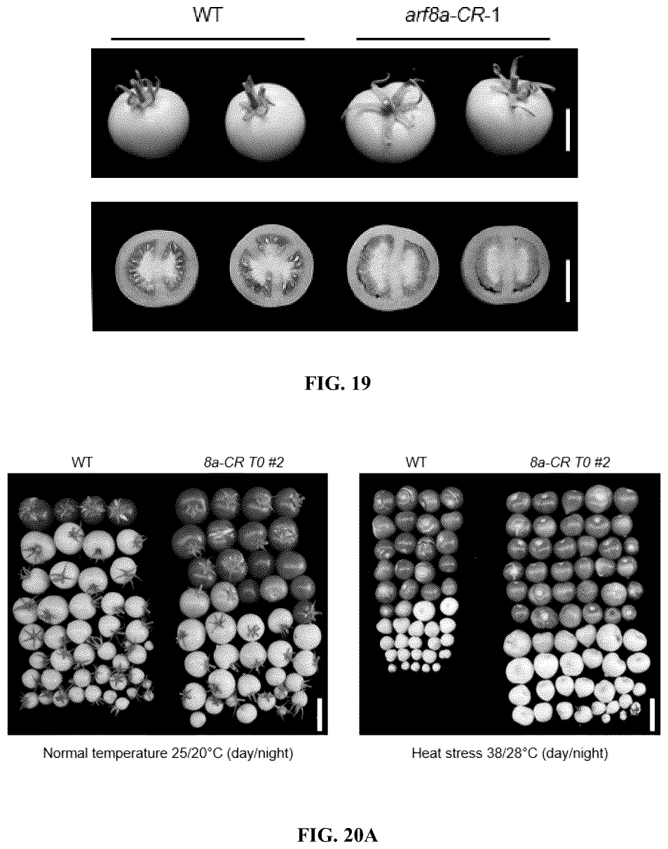

All T0 lines with ARF8A frameshift mutations showed strong parthenocarpic phenotype, similar to the P_{ARF8}:ARF8-NT transgenic lines. Four-week-old seedless fruits produced by T0 lines #2-#4 showed similar size and structure to same-age seeded wild type (WT) fruit (FIG. 18a). The overall plant phenotypes of the arf8a-CR mutants are similar to WT (FIG. 18b). The T0 #2 line was backcrossed to WT to separate the two different arf8a-CR alleles (i.e. 8a-CR-1, 8a-CR-2), generated in the same line by CRISPR/Cas9. The Cas9-gRNA transgene was also segregated out during this process. In the F2 generation, clean homozygous 8a-CR-1 and 8a-CR-2 lines without the CRISPR/Cas9 transgenes have been identified recently. 8a-CR-1 mutant showed the same parthenocarpic phenotype as the T0 plant (FIG. 19). The 8a-CR-2 fruit phenotype can be examined using the methods described herein.

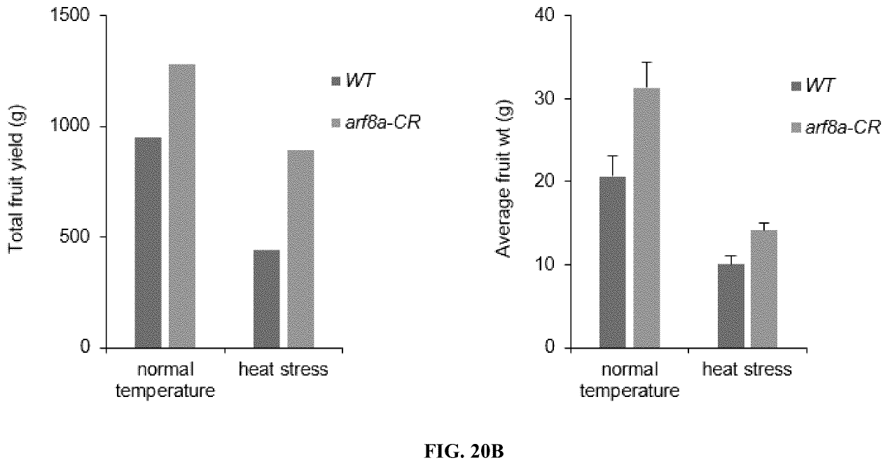

To assess fruit yield of the arf8A-CR mutants in response to extreme heat, arf8a-CR T0 #2 and WT plants were grown under normal and heat stress conditions. All plants (four plants each line) were first grown under normal conditions for 6 weeks to reach peak flowering stage. Then the plants were divided into two groups, one group was transferred to heat stress condition while the other group remained in normal condition. After eight weeks, all fruits were collected for weight measurements. The arf8a-CR T0 #2 line produced more fruits and had higher yield than WT in both conditions (FIGS. 20a and 20b). But the yield difference was more dramatic under heat stress condition, mainly due to more fruits produced by 8a-CR plants (FIG. 20b). In addition, the 8a-CR #2 fruits appeared to ripen faster than that of WT under normal condition (FIG. 20a). Future tests will be conducted using the clean homozygous arf8a-CR lines, and cold stress condition will also be tested. In summary, these results showed that applying CRISPR/Cas9 technology to target the PB1 domain sequence in an ARF transcription factor can be used to generate seedless fruit crops, with yield maintenance under heat stress.

TABLE 6
SIARFA CRISPR Alleles in T0 Lines

| T0 | SIARF8A mutations ** | | T0 |
|---|---|---|---|
| lines | Allele 1 | Allele 2 | phenotypes |
| #1 | not determined | not determined | poorly developed |
| | | | plant, discarded |
| #2* | gRNA1 has ↓T @cDNA nt2158- | gRNA1 has Δ of G (cDNA nt2158); | partially |
| | 2159; gRNA2 has Δ of CATC | gRNA2 has Δ of CATC (cDNA | |
| | (cDNA nt2510-2513) | nt2510-2513) | |
| | protein truncates @aa720 plus | protein truncates @aa720 plus extra | parthenocarpic |
| | extra 18aa•: this allele named 8a- | 93aa•: this allele named 8a-CR-2 | |
| | CR-1 | | |
| #3 | gRNA1 has ↓T @cDNA nt2158- | gRNA1 has Δ of TTGT (cDNA nt2156- | parthenocarpic, |
| | 2159; gRNA2 has Δ of CATC | 2159); gRNA2 has no mutation | |
| | (cDNA nt2510-2513) | | |
| | protein truncates @aa720 plus | protein truncates @aa719 plus extra | fewer leaflets |
| | extra 18aa• | 93aa• | |
| #4 | gRNA1 has ↓C @cDNA nt2158- | gRNA1 has Δ of gDNA nt6256-6286 | parthenocarpic, |
| | 2159; gRNA2 has disruption in a | and nt6329-6330; gRNA2 has | |
| | 24nt region (cDNA nt2498-2521) | disruption in a 24nt region (cDNA | |
| | resulted in 18nt Δ and 6nt | nt2498-2521) resulted in 18nt Δ and | |
| | rearrangement | 6nt rearrangement | |
| | protein truncates @aa719 plus | protein truncates @aa720 plus extra | fewer leaflets |
| | extra 18aa• | 82aa• | |
| #5 | to be determined | to be determined | parthenocarpic |
| #6 | gRNA1 has Δ of ATTTG (cDNA | gRNA1 has Δ of ACATTTG (cDNA | parthenocarpic, |
| | nt2154-2158); gRNA2 has Δ of T | nt2152-2158); gRNA2 has Δ of TCCA | |
| | (cDNA nt2514) | (cDNA nt2508-2511) | |
| | protein truncates @aa718 plus | protein truncates @aa717 plus extra | fewer leaflets |
| | extra 18aa• | 93aa• | |

*After backcrossing to WT, 8a-CR-1 and 8a-CR-2 alleles were segregated to generate homozygous lines individually
**Δ: deletion in cDNA (or gDNA) seq, # from ATG;
↓: insertion in cDNA seq, # from ATG;
aa•: extra random peptide and stop from the frame-shift mutation
ARF8A cDNA nt2158-2159 = gDNA nt6254-6255;
ARF8A PB1 domain: aa716-804

What is claimed is:

1. A genetically modified flowering plant that expresses a modified Auxin Response Factor 8A (ARF8A) or an ortholog thereof, wherein the modified ARF8A or an ortholog thereof comprises a mutation in a Phox and Bem1 (PB1) domain, and wherein the flowering produces ovary-derived, seedless fruit.

2. The genetically modified flowering plant of claim 1, wherein the mutation is one or more amino acid substitutions in the PB1 domain.

3. The genetically modified flowering plant of claim 1, wherein the mutation is a deletion of the PB1 domain or a portion thereof.

4. The genetically modified flowering plant of claim 1, wherein an interaction between the modified ARF8A or an ortholog thereof and an auxin responsive protein is reduced.

5. The genetically modified flowering plant of claim 4, wherein the auxin responsive protein is an Aux/IAA transcription repressor protein.

6. The genetically modified flowering plant of claim 1, wherein the modified ARF8A is Solanum lycopersicum ARF8A (SlARF8A) or an ortholog thereof.

7. The genetically modified flowering plant of claim 1, wherein the SlARF8A ortholog comprises Solanum lycopersicum SlARF8B, Arabidopsis thaliana AtARF8, Capsicum annuum CaARF8, Solanum melongena SmARF8, Cucumis sativus CsARF8, Malus domestica MdARF8, or Citrus sinensis CsARF8.

8. The genetically modified flowering plant of claim 7, wherein the SlARF8A ortholog is a polypeptide comprising a sequence having at least 90% identity to at least one of SEQ ID NOs: 83-89.

9. The genetically modified flowering plant of claim 5, wherein the ARF8A is SlARF8A and the Aux/IAA transcription repressor protein is Solanum lycopersicum IAA (IAA9).

10. The genetically modified flowering plant of claim 1, wherein the entire PB1 domain or a portion thereof is deleted.

11. The genetically modified flowering plant of claim 6, wherein the modified SlARF8A comprises a deletion in amino acids 716-804 of SEQ ID NO: 1.

12. The genetically modified flowering plant of claim 11, wherein amino acids 716-804 of SEQ ID NO: 1 or a portion thereof are deleted.

13. The genetically modified flowering plant of claim 12, wherein the modified SlARF8A is a truncated SlARF8A comprising amino acids 1-716 of SEQ ID NO: 1.

14. The genetically modified flowering plant of claim 6, wherein the modified SlARF8A comprises one or more amino acid substitutions at positions K721, D771, E773, D775 or D781 of SEQ ID NO: 1.

15. The genetically modified flowering plant of claim 6, wherein the modified SlARF8A ortholog comprises Solanum lycopersicum SlARF8B comprising a deletion in amino acids 715-803 of SEQ ID NO: 83; Arabidopsis thaliana AtARF8 comprising a deletion in amino acids amino acids 704-792 of SEQ ID NO: 84; Capsicum annuum CaARF8 comprising a deletion in amino acids 534-622 of SEQ ID NO: 85; Solanum melongena SmARF8 comprising a deletion in amino acids 756-844 of SEQ ID NO: 86; Cucumis sativus CsARF8 comprising a deletion in amino acids 727-816 of SEQ ID NO: 87; Malus domestica MdARF8 comprising a deletion in amino acids 716-804 of SEQ ID NO: 88; or Citrus sinensis CsARF8 comprising a deletion in amino acids 690-778 of SEQ ID NO: 89.

16. The genetically modified flowering plant of claim 15, wherein the modified SlARF8A ortholog comprises Solanum lycopersicum SlARF8B and wherein amino acids 715-803of SEQ ID NO: 83 or a portion thereof are deleted; wherein the modified SlARF8A ortholog comprises Arabidopsis thaliana AtARF8 and wherein amino acids 704-792 of SEQ ID NO: 84 or a portion thereof are deleted; wherein the modified SlARF8A ortholog comprises Capsicum annuum CaARF8 and wherein amino acids 534-622 of SEQ ID NO: 85 or a portion thereof are deleted; wherein the modified SlARF8A ortholog comprises Solanum melongena SmARF8 and wherein amino acids 756-844 of SEQ ID NO: 86 or a portion thereof are deleted; wherein the modified SlARF8A ortholog comprises Cucumis sativus CsARF8 and wherein amino acids 727-816 of SEQ ID NO: 87 or a portion thereof are deleted; wherein the modified SlARF8A ortholog comprises Malus domestica MdARF8 and wherein amino acids 716-804 of SEQ ID NO: 88 or a portion thereof are deleted; or wherein the modified SlARF8A ortholog comprises Citrus sinensis CsARF8 and wherein amino acids 690-778 of SEQ ID NO: 89 or a portion thereof are deleted.

17. The genetically modified flowering plant of claim 15, wherein the modified SlARF8A ortholog is a truncated SlARF8B comprising amino acids 1-714 of SEQ ID NO: 83; a truncated AtARF8 comprising amino acids 1-703 of SEQ ID NO: 84; a truncated CaARF8 comprising amino acids 1-533 of SEQ ID NO: 85; a truncated SmARF8 comprising amino acids 1-755 of SEQ ID NO: 86; a truncated CsARF8 comprising amino acids 1-726 of SEQ ID NO: 87; a truncated MdARF8 comprising amino acids 1-715 of SEQ ID NO: 88; or a truncated CsARF8 comprising amino acids 1-689 of SEQ ID NO: 89.

18. The genetically modified flowering plant of claim 6, wherein the modified SlARF8A ortholog comprises one or more amino acid substitutions at positions corresponding K721, D771, E773, D775, or D781 of SEQ ID NO: 1.

19. The genetically modified flowering plant of claim 1, wherein the plant is a genetically modified plant from the clade Angiospermae.

20. The genetically modified flowering plant of claim 13, wherein the plant is a genetically modified plant selected from the group consisting of: a Solanaceae plant, a Cucurbitaceae plant, an Ericaceae plant, a Rutaceae plant, a Vitaceae plant, an Anacardiaceae plant, a Lauraceae plant, a Moraceae plant, a Cactaceae plant, a Caricaceae plant, a Ebenaceae plant, a Myrtaceae plant, a Annonaceae plant, a Rhamnaceae plant, and a Sapindaceae plant.

21. The genetically modified flowering plant of claim 20, wherein the plant is a genetically modified Solanaceae plant.

22. The genetically modified flowering plant of claim 21, wherein the plant is a genetically modified Solanum plant.

23. The genetically modified flowering plant of claim 21, wherein the Solanum plant is a tomato plant or an eggplant.

24. The genetically modified flowering plant of claim 1, wherein fruit from the plant exhibits placental growth that is at least 80% of the placental growth exhibited by fruit from a wildtype plant of the same species.

25. The genetically modified flowering plant of claim 1, wherein the plant exhibits increased yield as compared to a control.

26. The genetically modified flowering plant of claim 1, wherein the plant exhibits increased yield under temperature stress conditions as compared to a control.

27. The genetically modified flowering plant of claim 1, wherein the temperature stress is heat stress or cold stress.

28. The genetically modified flowering plant of claim 1, wherein one or both alleles of an arf8a gene or an ortholog thereof comprise a mutation in the genomic sequence encoding the PB1 domain.

29. The genetically modified flowering plant of claim 28, wherein the mutation is an insertion, a deletion or substitution of one or more nucleic acids in the genomic sequence encoding the PB1 domain.

30. The genetically modified flowering plant of claim 28, wherein the amount and/or activity of ARF8A produced by the plant is increased.

31. The genetically modified flowering plant of claim 1, wherein the plant comprises an expression construct, and wherein the expression construct comprises a promoter operably linked to a recombinant nucleic acid sequence encoding ARF8A or an ortholog thereof comprising a mutation in the Phox and Bem1 (PB1) domain.

32. The genetically modified flowering plant of claim 31, wherein the mutation is a deletion of the PB1 domain or a fragment thereof.

33. A method of producing the genetically modified plant of claim 1, wherein the method comprises
a) modifying one or both alleles of an arf8a gene in one or more flowering plant cells to introduce a mutation into the nucleic acid sequence of the PB1 domain; and
b) generating one or more flowering plants from the one or more flowering plant cells.

34. The method of claim 33, wherein the genome of the one or more flowering plant cells is modified by contacting the one or more flowering plant cells with an expression construct comprising a nucleic acid sequence encoding ARF8 or an ortholog thereof comprising a mutation in the Phox and Bem1 (PB1) domain.

35. The method of claim 33, wherein the genome of the one or more flowering plant cells is modified by using Clustered Regularly Interspaced Short Palindromic Repeats (CRISPR) gene editing.

36. The method of claim 33, further comprising obtaining fruit from the one or more flowering plants.

37. The method of claim 33, further comprising crossing the genetically modified flowering plant with a wildtype plant of the same species.
